(19) United States
(12) Patent Application Publication
(10) Pub. No.: US 2026/0132230 A1
(43) Pub. Date: May 14, 2026

(54) LIPID-POLYMER COMPOUNDS, COMPOSITIONS, AND USES THEREOF

(71) Applicant: Mirus Bio LLC, Madison, WI (US)

(72) Inventors: Nicholas Rossi, Madison, WI (US); Keith Parsons, Madison, WI (US); James Ludtke, Madison, WI (US); Laura Juckem, Madison, WI (US)

(21) Appl. No.: 19/120,683

(22) PCT Filed: Oct. 10, 2023
(86) PCT No.: PCT/US2023/076498

§ 371 Date: Apr. 11, 2025

Related U.S. Application Data
(60) Provisional application No. 63/379,031, filed on Oct. 11, 2022.

(51) Int. Cl. C08F 8/30; A61K 9/51; A61K 31/7105; A61K 47/69; C08F 8/34; C08F 8/42; C12N 15/88

(52) U.S. Cl. CPC C08F 8/30; A61K 9/5138; A61K 31/7105; A61K 47/6933; C08F 8/34; C08F 8/42; C12N 15/88; C08F 2810/40

(57) ABSTRACT

Described herein are compounds comprising a lipid connected to a backbone of a polymer with functional groups. Also described herein is the use of such reagents for delivering nucleic acids to a cell.

### CROSS-REFERENCE

This application claims the benefit of U.S. Provisional Application No. 63/379,031, filed Oct. 11, 2022, which is incorporated by reference herein in its entirety.

### BACKGROUND OF THE INVENTION

The control of living processes may be mediated through nucleic acids. Nucleic acids can encode proteins which, as enzymes, hormones, and other regulatory factors, may carry out the processes which enable living organisms to function. Nucleic acids may also encode for regulatory sequences which control the expression of proteins. Because of their central role in living organisms, nucleic acids may be an ideal therapeutic target.

One factor that could limit nucleic acid-based therapies is the ability to deliver nucleic acids to the appropriate compartment of the cells. Nucleic acids can be fragile molecules which may be highly negatively charged (one negative charge per phosphate group) and which may be readily cleaved by nucleases present both in extracellular fluids and intracellular compartments. While some attempts to encapsulate or otherwise stabilize nucleic acids with proteins, peptides, polymers, lipids, liposomes, and lipid nanoparticles have shown some promise, there remains a need to identify safe, non-toxic means for stabilizing nucleic acid molecules in biological systems, e.g., to enhance therapeutic efficacy of such therapies. One strategy for achieving this goal, among others, is the development of lipid-polymer conjugates, which can, for example, interact with (e.g., stabilize) nucleic acid molecules, form a liposome or lipid nanoparticle, and/or insinuate into a lipid membrane, e.g., as a reporter or functional handle within a cell membrane. Additional compositions and methods related to lipid-polymer compounds are described herein.

### SUMMARY OF THE INVENTION

The present disclosure provides lipid-polymer compounds (alternately, “lipid-polymer conjugates”) that may be useful in various applications, such as delivery of biologically active compounds (e.g., nucleic acid molecules) into cells in biological systems, for instance, in in vitro cell transfection research. Lipid-polymer conjugates may comprise one or more lipid compounds conjugated to a polymeric backbone. The present disclosure also provides methods of making such compounds, which may have various applications, such as treating a disease or for use in gene therapy applications.

In an aspect, described herein is a compound comprising a lipid; and a stimulus-responsive unit. More examples of the lipid and the stimulus-responsive unit elements are described below.

In an aspect, described herein is a compound comprising a lipid; a linker comprising a stimulus-responsive unit; and a polymer, and the linker connects the lipid to a backbone of the polymer. In some embodiments, the polymer may comprise, for example, at least 3 monomeric units, where the at least 3 monomeric units comprise a C₁₋₂₀ heteroalkyl side-chain. In some embodiments, the stimulus-responsive unit is a temperature-responsive unit, a pH-responsive unit, a light-responsive unit, or a chemical-responsive unit. In some embodiments, the temperature-responsive unit comprises a lower crystalline solution temperature (LCST) from about 27° C. to about 35° C. In some embodiments, the temperature-responsive unit comprises a lower crystalline solution temperature (LCST) at about 27° C., about 28° C., about 29° C., about 30° C., about 31° C., about 32° C., about 33° C., about 34° C., or about 35° C. In some embodiments, the temperature-responsive unit comprises poly(N-isopropylacrylamide), poly(N-n-propylacrylamide), poly(N-methyl-N-n-propylacrylamide), poly(N,N-diethylacrylamide), poly(N-isobutylacrylamide), poly(N-sec-butylacrylamide), poly(N-n-butylacrylamide), poly(N-isobutylacrylamide), hydroxypropylcellulose, poly(N-vinylcaprolactam), poly-2-isopropyl-2-oxazoline, or polyvinyl methyl ether, or a combination thereof. In some embodiments, the temperature-responsive unit comprises 2-250 monomeric units. More examples of the lipid and the polymer are described below.

In an aspect, described herein is a compound comprising a lipid connected to a backbone of a polymer. The polymer may comprise, for example, at least 3 monomeric units, where the at least 3 monomeric units comprise a C₁₋₂₀ heteroalkyl side-chain. In some instances, a polymer comprises 4 or more (e.g., 10 or more, 50 or more) the monomeric units. The polymer may comprise about 400 or less (e.g., about 300 or less) the monomeric units. In some instances, a polymer comprises from about 10 to about 200 monomeric units. In certain examples, a polymer comprises from about 50 to about 150 monomeric units. A polymer may comprise a polyacrylate or a polyacrylamide. A polymer may comprise one or more acrylate side-chains and/or one or more acrylamide side-chains. In some embodiments, each monomeric comprises an acrylamide or an acrylate. A polymer may be a peptide or a non-peptide. As used herein, a polymer is generally a non-peptide. For example, the polymer may comprise no amino acids. In some embodiments, each monomeric unit is not an amino acid. The polymer may comprise a copolymer. For example, the polymer may comprise a block copolymer. In some instances, the block copolymer comprises a cationic or cation-forming monomeric unit. The block copolymer can be a random block copolymer. In some instances, the polymer is positively charged in neutral aqueous solution. A polymer may comprise a pK^{b} from about 2 to about 12 (e.g., from about 4 to about 11). More examples of the lipid and the polymer are described below.

In another aspect, provided herein is a compound according to Formula I:

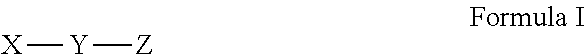

or a pharmaceutically acceptable salt thereof; wherein:
X is a lipid;
Y is a polymer comprising 3 or more monomeric units, wherein each the monomeric unit comprises a C₁₋₂₀ heteroalkyl side-chain; and
Z is an unsubstituted or substituted functional group;
wherein the lipid is covalently bonded to the polymer via a backbone of the polymer.

In some embodiments, the lipid comprises a steroid or a fatty acid. In some embodiments, the steroid comprises a sterol or a stanol. In some embodiments, the steroid comprises the sterol. In some embodiments, the sterol comprises a cholesterol. In some embodiments, the fatty acid comprises a saturated fatty acid, a monounsaturated fatty acid, a polyunsaturated fatty acid, or a combination thereof. In some embodiments, the fatty acid comprises an oleic acid, or an ester thereof. In some embodiments, the lipid is hydrophobic. In some embodiments, the lipid is amphiphilic. In some embodiments, the lipid has an octanol:water coefficient (log(K_{OW})) of about 2 or more. In some embodiments, provided herein is a compound (e.g., of Formula I), wherein the lipid (X) has a structure of Formula X-A, Formula X-B, or Formula X-C:

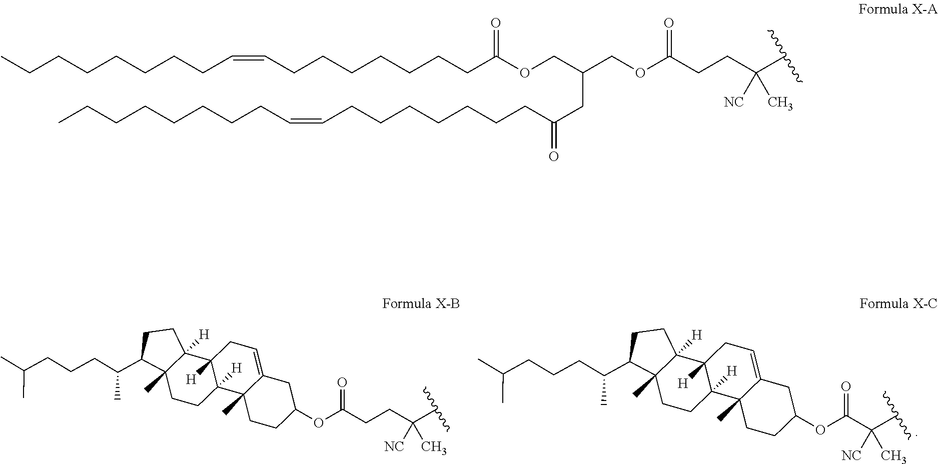

In some embodiments, provided herein is a compound (e.g., of Formula I), wherein the polymer (Y) has a structure of Formula Y-A, Formula Y-B, Formula Y-C, or Formula Y-D:

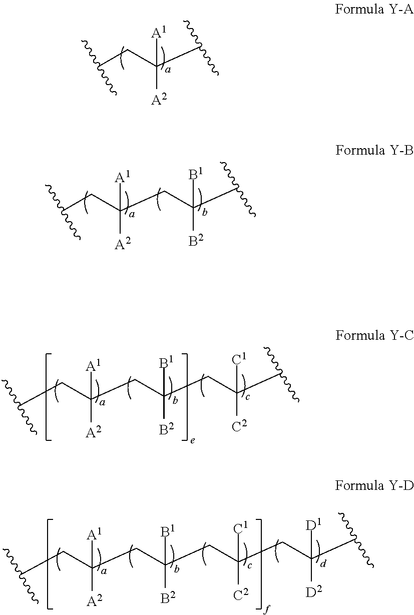

or a pharmaceutically acceptable salt thereof; wherein:
each of A¹, B¹, C¹, and D¹ is independently hydrogen or methyl;
each of A², B², C², and D² is independently unsubstituted or substituted C₁₋₂₀ heteroalkyl;
each of a, b, c, d, e, and f is independently an integer from 0 to 200, provided that the total number of the monomeric units is 3 or more;
wherein each the substituted C₁₋₂₀ heteroalkyl is independently substituted with a cycle that is an unsubstituted or substituted cycloalkyl, unsubstituted or substituted heterocyclyl, unsubstituted or substituted aryl, or unsubstituted or substituted heteroaryl.

In some embodiments, the polymer comprises from about 4 to about 400 the monomeric units. In some embodiments, the polymer comprises from about 10 to about 200 the monomeric units. In some embodiments, the polymer comprises from about 50 to about 150 the monomeric units. In some embodiments, the polymer comprises a polyacrylate or a polyacrylamide. In some embodiments, each of the A², B², C², and D² independently comprises an acrylate or an acrylamide. In some embodiments, each of the monomeric unit has the structure of one of the following formulae:

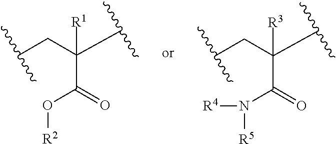

or a pharmaceutically acceptable salt thereof wherein:
each of R¹ and R³ is independently hydrogen or methyl;
each R² is hydrogen, C₁₋₆ alkyl, C₇₋₂₀ aralkyl, C₁₋₂₀ heteroalkyl, or polyethylene glycol chain containing 1 to 100 ethylene glycol monomers; wherein each the C₁₋₆ alkyl, C₇₋₂₀ aralkyl, and C₁₋₂₀ heteroalkyl is unsubstituted or substituted with one or more groups, wherein each of the one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, or —S(O)₂OH;
each of R⁴, and R⁵ is independently hydrogen, C₁₋₆ alkyl, C₇₋₂₀ aralkyl, or C₁₋₂₀ heteroalkyl; wherein each the C₁₋₆ alkyl, C₇₋₂₀ aralkyl, and C₁₋₂₀ heteroalkyl is unsubstituted or substituted with one or more groups, wherein each of the one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH—, —S(O)₂CH₃, or —S(O)₂OH; or a pharmaceutically acceptable salt thereof.

In some embodiments, each of the monomeric unit independently comprises:

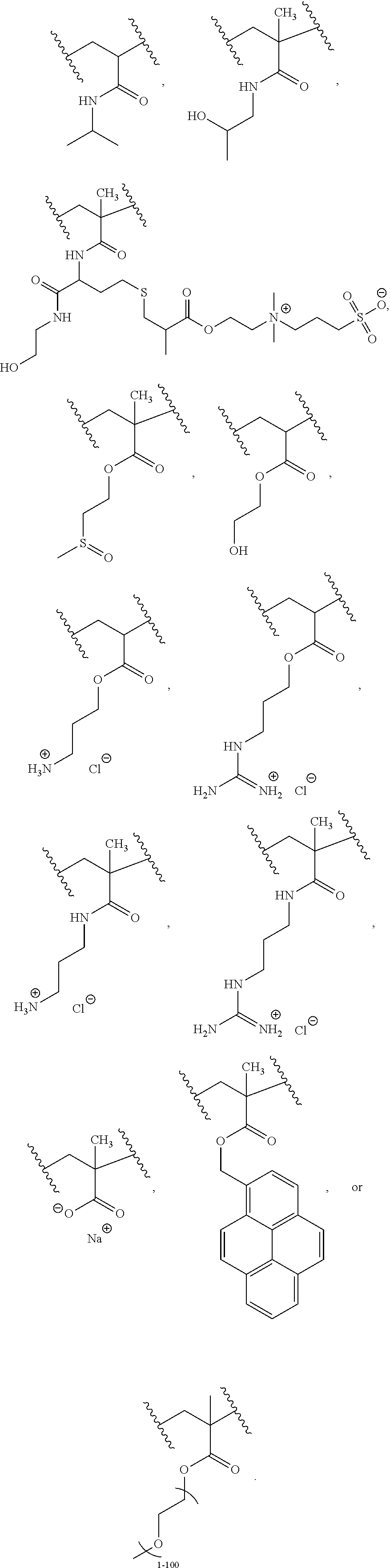

In some embodiments, the functional group is a thiol or a sulfide. In some embodiments, the functional group is the thiol. In some embodiments, the functional group is the sulfide. In some embodiments, the sulfide is SR⁶, and wherein R⁶ is a group consisting of 1 to about 200 atoms selected from hydrogen, halogen, C, N, O, and S. In some embodiments, the sulfide is SR⁶, and wherein R⁶ comprises a reactive group, a charged group, a detectable group, a peptide group, a capping group, or a combination thereof. In some embodiments, the reactive group comprises an azide or an alkyne. In some embodiments, the charged group comprises one or more cationic groups. In some embodiments, the one or more cationic groups comprise a cyclic amine, primary amine, guanidine, or a combination thereof. In some embodiments, the detectable group comprises a fluorophore, a dye, a FRET donor or acceptor. In some embodiments, the capping group is an inert group. In some embodiments, the functional group is selected from:

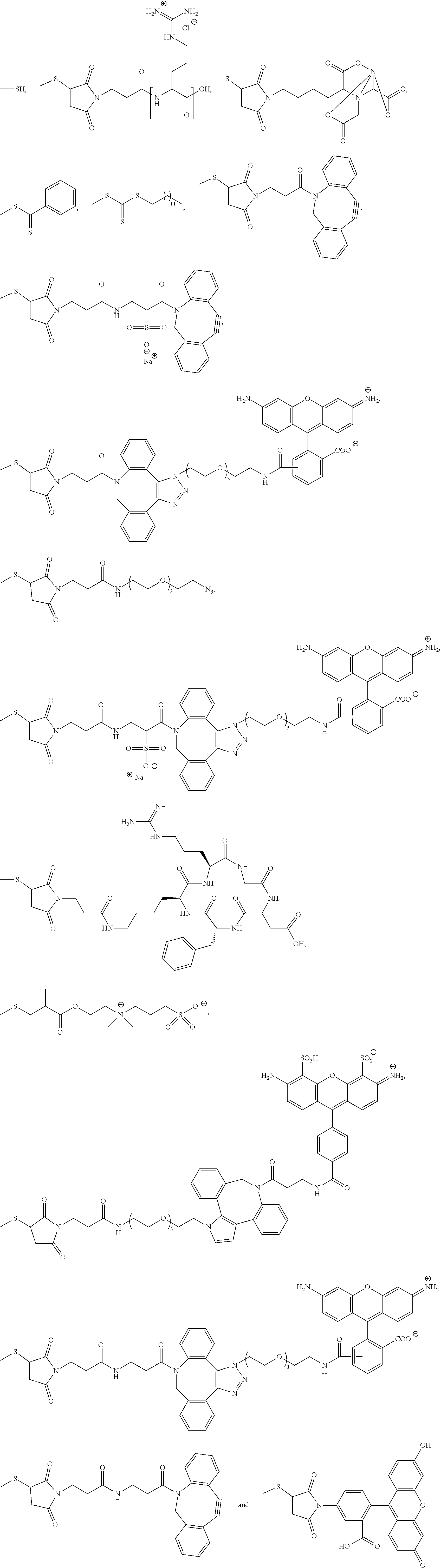

wherein the functional group is bonded to the polymer via a sulfur atom.

In some embodiments, the compound is configured to encapsulate or complex with nucleic acids in aqueous solution. In some embodiments, the compound is substantially non-toxic. In some embodiments, the compound is biodegradable. In some embodiments, the compound comprises a molecular weight from about 1 kilodaltons (kDa) to about 100 kDa.

In another aspect, described herein is a nanoparticle comprising a compound or PLip disclosed herein, wherein the nanoparticle is configured for encapsulation or complexation of nucleic acids. In some embodiments, the nanoparticle is configured for the encapsulation or the complexation of the nucleic acids at a 0.3:1 to 100:1 (weight:weight or w/w) ratio. In some embodiments, the encapsulation or the complexation of the nucleic acids increases a half-life of the nucleic acids by at least 2-fold in aqueous or physiological conditions. In some embodiments, the nuclease digestion of the nucleic acid is inhibited by the encapsulation or complexation. In some embodiments, the encapsulation or the complexation of the nucleic acids generates a transfection reagent with an average size of about 20 nm to about 2000 nm. In some embodiments, the complexation comprises adsorption of at least a subset of the nucleic acids to a surface of the nanoparticle. In some embodiments, the encapsulation or complexation of the nucleic acids generates a transfection reagent configured for cellular uptake. In some embodiments, the cellular uptake comprises endocytosis.

In another aspect, provided herein is a transfection reagent comprising a nanoparticle described herein (e.g., comprising a compound disclosed herein) with genetic information (e.g., one or more nucleic acid molecules) encapsulated therein. In some embodiments, the nucleic acid comprises deoxyribonucleic acid (DNA), ribonucleic acid (RNA), locked nucleic acid (LNA), peptide nucleic acid (PNA), or any combination thereof. In some embodiments, the nucleic acid comprises about 1 kilobase pairs (kb) to about 100 kb. In some embodiments, the nucleic acid comprises about 2 kb to about 20 kb. In some embodiments, the nucleic acid comprises about 5 kb to about 15 kb. In some embodiments, the nucleic acid comprises about 8 kb to about 12 kb. In some embodiments, the nucleic acid comprises about 10 kb. In some embodiments, provided herein is a transfection reagent comprising an aqueous solubility of at least 5 μg/mL. In some embodiments, provided herein is a transfection reagent comprising an aqueous solubility of about 5 μg to about 5 mg/mL. In some embodiments, provided herein is a transfection reagent comprising an aqueous solubility of about 10 μg/mL to about 50 μg/mL.

In another aspect, provided herein is a method for transfecting a cell, the method comprising: (a) providing a transfection reagent comprising a compound disclosed herein and a nucleic acid, and (b) contacting the cell with the transfection reagent, wherein the contacting is under conditions suitable for entry of the nucleic acid into the cell. In some embodiments, step (a) comprises contacting the compound with the nucleic acid under conditions sufficient to form the transfection complex. In some embodiments, the conditions sufficient to form the transfection complex comprise conditions sufficient for ionotropic gelation. In some embodiments, the nucleic acid comprises deoxyribonucleic acid (DNA), ribonucleic acid (RNA), locked nucleic acid (LNA), peptide nucleic acid (PNA), or any combination thereof. In some embodiments, the transfection complex comprises a positive charge under the conditions suitable for entry of the nucleic acid into the cell. In some embodiments, the contacting is for less than 24 hours. In some embodiments, the cell comprises an animal cell, a plant cell, a fungal cell, a bacterial cell, or any combination thereof.

In another aspect, provided herein is a pharmaceutical composition comprising a nanoparticle disclosed herein (e.g., comprising a compound disclosed herein (e.g., of Formula I)), and a bioactive molecule. In some embodiments, the nanoparticle is a lipid nanoparticle. In some embodiments, the nanoparticle is covalently bonded to the bioactive molecule. In some embodiments, the nanoparticle is ionically bonded to the bioactive molecule. In some embodiments, the nanoparticle encapsulates the bioactive molecule. In some embodiments, the bioactive molecule comprises a nucleic acid molecule. In some embodiments, the nucleic acid molecule comprises RNA or DNA. In some embodiments, the nucleic acid molecule comprises mRNA, siRNA, or tRNA. In some embodiments, the bioactive molecule comprises a therapeutic agent. In some embodiments, the therapeutic agent is a chemotherapeutic, a radiotherapeutic, an oligonucleotide, or an oligopeptide. In some embodiments, the pharmaceutical composition further comprises a pharmaceutically acceptable excipient.

In still another aspect, provided herein is a method of treating a condition or disease in a subject in need thereof, comprising administering to the subject a therapeutically effective amount of a transfection reagent or pharmaceutical composition disclosed herein. In some embodiments, the transfection reagent or pharmaceutical composition is administered to the subject by injection.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the ¹H NMR spectrum of the CPCPA-diol of Example S1.

FIG. 2 shows the ¹H NMR spectrum of the CPCPA-dilinoleyl RAFT agent of Example S2.

FIG. 3 shows the ¹H NMR spectrum of the CPCPA-cholesterol RAFT agent of Example S3.

FIG. 4 shows the ¹H NMR spectrum of the cationic diacyl PLip of Example S8 (a).

FIG. 5 shows the ¹H NMR spectrum of the P(HPMA) PLip of Example S9.

FIG. 6 shows the percent encapsulation of DNA for a lipid nanoparticle formulated with a cationic PLip of Example B1(a).

FIG. 7 shows the effect on LNP binding that results from incorporating 0.5 mol % PLip 7 into the LNP formulation of Example B2(b).

FIG. 8 shows the dynamic light scattering intensity distribution of LNPs comprising PLips 6 and 8.

FIG. 9 shows the anti-spike protein IgG in mice at day 14 and day 35 after LNP-encapsulated mRNA delivery, where each bar represents an individual mouse.

FIG. 10 shows the dose response priming of Jurkat cells with PLip 12 insertion.

FIG. 11 shows the fluorescence measurements at different temperatures following insertion of the fluorescent PLip 13 into the cytoplasmic membrane of 293F cells.

FIG. 12 shows the radius and normalized fluorescence of LNPs 14-19 after mixing with azidefluor-488.

FIG. 13 shows a comparison of 293F cells transfected by cationic PLips 20-22 alone.

FIG. 14 shows comparison of 293F cells transfected by TransIT®-Jurkat, and POLY1, with and without cationic PLips 20-22.

FIG. 15 shows the effect of temperature cycling on LNP radius for 2 LNPs containing 0.6 and 1.2 mol % of temperature-sensitive PLip 23, and 1 control LNP containing no PLip 23.

FIG. 16 shows the effects of temperature cycling on the normalized intensity data from dynamic light scattering analysis (number of particles).

FIG. 17A shows percent full capsids over time (after transfection) in the presence or absence of a stabilizing PLip when evaluating complex formation. FIG. 17B shows genomes counts over time (after transfection) in the presence or absence of the stabilizing PLip when evaluating complex formation.

FIG. 18A shows complex formation concentrations in the absence of the stabilizing PLip measured by dynamic light scattering (DLS). FIG. 18B shows complex formation concentrations in the presence of the stabilizing PLip measured by DLS.

FIG. 19A shows complex formation concentrations measured by genomes titer and percent full capsids over time in the presence of a stabilizing PLip. FIG. 19B shows another example of complex formation concentrations measured by genomes titer and percent full capsids over time in the presence of the stabilizing PLip.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION OF THE INVENTION

The control of living processes is mediated through nucleic acids. Nucleic acids encode proteins which, as enzymes, hormones, and other regulatory factors, and carry out the processes which enable living organisms to function. Nucleic acids also encode for regulatory sequences which control the expression of proteins. It is thought that many diseases could be controlled by the manipulation of nucleic acids in living organisms.

A factor limiting therapies based on nucleic acid manipulation is the ability to deliver nucleic acids to the appropriate compartment of the cells. Nucleic acids are fragile molecules which are highly negatively charged (one negative charge per phosphate group) and which are readily cleaved by nucleases present both in extracellular fluids and intracellular compartments. As a highly charged molecule, it will not cross the lipid membranes surrounding the cell, nor can it readily escape from endosomal compartments involved in the uptake of macromolecules into cells. Even RNA interference (RNAi) molecules, although smaller in molecular weight, face significant problems with stability and uptake.

The efficient delivery of biologically active compounds to the intracellular space of cells has been attempted by the use of a wide variety of vesicles. Liposomes are microscopic vesicles that comprise amphipathic molecules, which contain both hydrophobic and hydrophilic regions. In addition, biologically active compounds can be delivered to the intracellular space of a cell via a liposome-like structure, e.g., a lipid nanoparticle (LNP). LNPs can be efficient for encapsulating a broad variety of biologically active compounds, e.g., nucleic acids (e.g., mRNA, microRNA, siRNA, and the like). Liposomes and LNPs may also be useful in delivering a broad variety of biologically active compounds, e.g., nucleic acids, to the intracellular space of a cell. The development, synthesis, and characterization of lipid-polymer compounds, as well as their various uses in treating and/or detecting processes in biological systems are described herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present invention relates to lipid-polymer compounds (alternately, “lipid-polymer conjugates,” or simply, “compounds”) comprising one or more lipid compounds conjugated to a polymeric backbone, which can be used in the delivery of nucleic acid to cells in biological systems, for instance, in in vitro cell transfection research. The invention also relates to methods of making such compounds and potentially to gene therapies using such compounds.

The present invention also provides a compound to promote the transfer of genetic material (e.g., nucleic acid molecules) into animal cells via a complex comprising nucleic acids and polymers containing ionic or non-ionic side-chain moieties. The lipid-polymer compounds described herein may be used, for example, as nucleic acid transfection agents. In some instances, the lipid-polymer conjugates or lipid polymer compounds described herein are used in conjunction with endosomolytic lipids to transfect nucleic acids into cells.

Some methods described herein may be useful for the purposes of altering the expression of one or more genes in a cell or cells.

Also provided herein, in some instances, are compositions and compounds that can facilitate delivery of nucleic acids to an animal cell (or cells) in vitro and/or in vivo. Nucleic acids may comprise a double stranded structure having a nucleotide sequence substantially identical to parts of an expressed target nucleic acid within the cell. Further, the use of a lipid connected to a backbone of a polymer as provided herein can significantly increase nucleic acid transfer efficiency. A nucleic acid may then alter expression of a selected endogenous nucleic acid.

The lipid connected to a backbone of a polymer, as described herein, may be used to assist transfection of DNA, RNA, mRNA, or RNAi into a cell. The nucleic acid may then alter the cell's natural process.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are generally intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” Similarly, the terms “between,” “from,” “to,” when referring to a range is intended to be inclusive of the entire range. For example, “between 0 and 5” is intended to include both 0 and 5, as well as the integers (or non-integer numbers, where appropriate) between (e.g., 1-4). Similarly, a range “from 0 to 5” is intended to include 0, 1, 2, 3, 4, and 5.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about” when referring to a number or a numerical range generally means that the number or numerical range referred to is an approximation within experimental variability (or within statistical experimental error), and the number or numerical range may vary from, for example, from 1% to 15% of the stated number or numerical range. As used herein, the term “about” generally refers to ±10% of a stated number or value. For small numbers (e.g., 1 to 10), the term “about” may refer to +0.5 of that number. For example, if a variable has a value that is “about 2,” it should be understood that “about 2” refers to the range of 1.5 to 2.5 (inclusive). Similarly, if a variable has a value that is “about 4.5,” it should be understood that this term refers to the range from 4 to 5.

As used herein, C₁-Cₓ can include C₁-C₂, C₁-C₃, . . . , C₁-Cₓ. By way of example only, a group designated as “C₁-C₄” indicates that there are one to four carbon atoms in the moiety, i.e., groups containing 1 carbon atom, 2 carbon atoms, 3 carbon atoms, or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group generally refers to an aliphatic hydrocarbon group. The alkyl group may be branched or a straight chain. An “alkyl” group may comprise 1 to 10 carbon atoms, i.e., a C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. Alkyl groups can be of any size. In some examples, an alkyl group is a carbon chain of about 3 to about 36 carbons in length, wherein one or more (e.g., 1, 2, 3, 4, 5, or 6) bonds are double-bonds. An alkyl group may be a fatty acid (e.g., a C₆-C₃₀ alkyl or alkenyl chain).

An “alkoxy” group may refer to a (alkyl)O— group, where alkyl is as defined herein. In some embodiments, “alkoxy” refers to methoxy (—OCH₃), ethoxy (—OCH₂CH₃), and the like.

A “hydroxyalkyl” refers to an alkyl in which at least one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

An “aminoalkyl” may refer to an alkyl in which at least one hydrogen atom is replaced by an amine (—NH₂, —NHR, —NR₂, or —NR₃⁺). In some embodiments, aminoalkyl is a C₁-C₆ aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like.

The term “alkenyl” may refer to a type of alkyl group in which at least one carbon-carbon double bond is present. In one embodiment, an alkenyl group has the formula —C(H)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include, —CH═CH—, —CH═CH₂, —C(CH₃)═CH₂, —CH═CHCH₃, —C(CH₃)═CHCH₃, and —CH₂CH═CH₂. An alkene can be either cis (or “Z” configuration) or trans (or “E” configuration). An alkene may also comprise multiple double bonds, each of which is independently E or Z. Preference is given to cis alkenes or cis-polyalkenes. For example, a cis alkene can be an oleyl group (e.g., an oleic acid or ester).

The term “alkynyl” generally refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡C—, —C≡CH, —C≡CCH₃—C≡CCH₂CH₃, or —CH₂C≡CH.

The term “heteroalkyl” generally refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆ heteroalkyl. In one aspect, a heteroalkyl is a C₆-C₃₀ heteroalkyl. Heteroalkyl groups may include nitriles, amides, esters, ethers, amines, thioethers, thioesters, carbamates, carbonates, polyethers, polyamines, and the like. Heteroalkyl groups may include alkyl ethers (e.g., polyethers), alkyl esters (e.g., polyesters), alkyl amines (e.g., polyamines), alkyl amides (e.g., polyamides), or any combination thereof. A heteroalkyl group may comprise an acrylate or an acrylamide. In some examples, a heteroalkyl group comprises an ether or a polyethylene glycol (PEG) group, wherein the PEG group comprises 2 to 100 monomeric units. By way of non-limiting example, each of A1-A12 may be regarded as having heteroalkyl side-chains.

The term “aromatic” generally refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” generally refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” generally refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₂₄ aryl. In some embodiments, “aryl” refers to a polycyclic aromatic carbocycle having two or more (e.g., 2, 3, 4, 5, 6, or 7) conjugated aromatic rings. Examples include, but are not limited to, phenanthrenes, anthracenes, pyrenes, benzopyrenes, coronenes, and the like. Depending on the structure, an aryl group may be a monovalent radical (i.e., an aryl group) or a divalent radical (i.e., an arylene group).

The term “cycloalkyl” may refer to a monocyclic or polycyclic aliphatic, non-aromatic radical, wherein each of the atoms forming the ring (i.e., skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[0.1.1]pentyl. In some embodiments, a cycloalkyl is a C₃-C₆ cycloalkyl. In some embodiments, a cycloalkyl is a C₃-C₄ cycloalkyl.

The term “halo” or, alternatively, “halogen” or “halide” generally refers to fluoro, chloro, bromo or iodo. In some instances, halo may refer to fluoro, chloro, or bromo. As used herein, the term “halo” is not intended to be numerically limiting. For example, a haloalkyl group may contain 1, 2, 3, or more halogen groups. A haloalkyl group can be, for example, —CHF₂, —CH₂F, or —CF₃.

The term “fluoroalkyl” generally refers to an alkyl in which one or more hydrogen atoms are replaced by a fluorine atom. In one aspect, a fluoroalkyl is a C₁-C₆ fluoroalkyl. Examples of fluoroalkyl groups include, but are not limited to, —CH₂F, —CHF₂, —CF₃, —CH₂CH₂F, —CH₂CF₃, —CF₂CF₃, and the like.

The term “heterocycle” or “heterocyclic” generally refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” or, alternatively, “heteroaromatic” generally refers to an aryl group that includes one or more ring heteroatoms selected from nitrogen, oxygen, and sulfur. Illustrative examples of heteroaryl groups include, but are not limited to, monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl.

A “heterocycloalkyl” group generally refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In some embodiments, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” generally refers to a chemical bond between two atoms or two moieties when the atoms joined by the bond are considered to be part of a larger substructure. In one aspect, when a group described herein is a bond, the referenced group is absent thereby allowing a bond to be formed between the remaining identified groups.

The term “moiety” generally refers to a specific segment or functional group of a molecule. Chemical moieties are often recognized chemical entities embedded in or appended to a molecule.

The term “substituted” generally refers to a group wherein one or more hydrogen atoms are replaced with a substituent, for example, a substituent that is selected from the group consisting of halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthiol, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄ alkyl), —C(═O)NH₂, —C(═O)NH—(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂C₁-C₄ alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O). A group may also be “optionally substituted,” meaning the group may be unsubstituted, or substituted as described above.

The term “acrylates” generally refers to the salts, esters, or conjugate bases of acrylic acid. These acrylates (CH₂═CHCO₂R or CH₂═CHCO₂H) contain vinyl groups, which are susceptible to polymerization and the carboxylate group carries myriad functionalities. Modified acrylates include, but are not limited to, methacrylates (CH₂═C(CH₃)CO₂R or CH₂═C(CH₃)CO₂H) and cyanoacrylates (CH₂═C(CN)CO₂R or CH₂═C(CN)CO₂H). As used herein, the term “acrylates” include such modified acrylates, including “methacrylates.” As used herein, the term “acrylates” include “methacrylates.”

The term “acrylamide” generally refers to a vinyl-substituted primary, secondary, or tertiary amide (CH₂═CHC(O)NH₂, CH₂═CHC(O)NHR or CH₂═CHC(O)NR₁R₂). Modified acrylamides include, but are not limited to, methacrylamides (CH₂═C(CH₃)C(O)NH₂, CH₂═C(CH₃)C(O)NHR, or CH₂═C(CH₃)C(O)NR₁R₂) and cyanoacrylamides (CH₂═C(CN)C(O)NH₂, CH₂═C(CN)C(O)NHR or CH₂═C(CN)C(O)NR₁R₂). As used herein, the term “acrylamides” include such modified acrylamides, including “methacrylamides.” As used herein, the term “acrylamides” include “methacrylamides.”

“Polymer” generally refers to molecules that are built up by repetitive bonding together of smaller units, called monomers. As used herein, the term “polymer” may include both oligomers, which can have two to about 80 monomers, and polymers having more than 80 monomers. A polymer may comprise, for example, 4 or more monomeric units (e.g., 5 or more, 10 or more, 20 or more, 50 or more, 75 or more, 100 or more, 200 or more, 300 or more, or 400 or more monomeric units or monomers). A polymer may comprise, for example, no more than about 1000 monomeric units or monomers (e.g., no more than about 500, no more than about 400, no more than about 300, no more than about 200, no more than about 150, no more than 100, or no more than about 50 monomeric units or monomers). In some examples, a polymer has between about 10 and about 200 monomeric units (e.g., about 20 to about 200, about 50 to about 200, about 75 to about 200, or about 100 to about 200 monomers). A polymer can be linear, branched network, star, comb, or ladder types of polymer. A polymer can be a homopolymer in which a single monomer is used or can be a copolymer in which two or more monomers are used. Types of copolymers include alternating, random, block, and graft. A “main chain” of a polymer or “a backbone chain” of a polymer may refer to the longest series of covalently bonded atoms that together create a continuous chain of a given molecule. The main chain of a polymer or the backbone chain of a polymer can be composed of the atoms whose bonds are required for propagation of polymer length in step-growth or chain-growth polymerization. The side chain of a polymer can be composed of the atoms whose bonds are not required for propagation of polymer length. To those skilled in the art of polymerization, there are several categories of polymerization processes that can be utilized in the described process. In some embodiments, polymers described herein have heteroalkyl side-chains (e.g., acrylate or acrylamide side-chains). A polymer may comprise an alkyl backbone. A polymer may comprise an alkenyl backbone. A polymer may comprise a heteroalkyl backbone. Examples of heteroalkyl backbones include, but are not limited to, polyethers (e.g., polyethylene glycol, or PEG) and polyamines (e.g., polyethyleneimine, or PEI).

“Monomer” generally refers to the building blocks from which a polymer is constructed. As used herein, a “monomer” can be a divalent chemical unit, wherein each valency of the unit is completed by bonding to (e.g., via polymerization) to an adjacent monomer (thereby creating a polymer), or to a terminal group (e.g., a lipid or a functional group as described herein). Monomers described herein may be attached to adjacent monomers or terminal groups via the backbone. A monomer may be bonded to an adjacent group (e.g., a second monomer, a lipid, or a functional group) via a bond, or via a linker (e.g., a C₁₋₂₀ alkyl linker, which is optionally substituted with an amine, an amide, an ether, an ester, a carbonyl, a carbamate, a carbonate, and the like). For example, a polymer may be attached to a lipid or a functional group via a linker, wherein the linker is a C₁₋₂₀ alkyl ester (e.g., a C₁₋₆ alkyl ester).

“Side chains” or “side-chains” generally refers to groups of atoms within a monomer that are bonded to the backbone, which do not contribute to the chain propagation and may optionally possess additional functionality. For example, a side-chain having an amino group (e.g., a linear, branched, cyclic, or primary, secondary, tertiary, or quaternary amine) which can form a cation. In some embodiments, side-chains having basic or cationic properties interact with the acidic or anionic phosphate backbone of a nucleic acid molecule, thereby enhancing the capacity of the monomer (or compound comprising such monomers) to bind the nucleic acid. In some embodiments, a cationic or basic side-chain interacts with an anionic phosphate group of a nucleic acid molecule, thereby forming a net-neutral (or more neutral relative to an unbound nucleic acid) molecule, which may then pass through cell membranes. In some embodiments, a side-chain is specifically designed to enhance transport of charged molecules (e.g., nucleic acid molecules) into and out of cells. In some embodiments, a side-chain is not cationic. In some embodiments, a side-chain is a heteroalkyl group that modulates one or more properties of the lipid-polymer conjugate (e.g., toxicity, solubility, stability, non-specific (e.g., protein plasma) binding, specific (e.g., nucleic acid) binding, polarity, detectability, etc.). A monomer may not have a side-chain (e.g., a PEG or PEI group). In some embodiments, a polymer comprises repeating units of two or more monomers, thereby creating an A-B-A-B-A-B pattern of side-chains, wherein A and B represent the same or different side-chains.

“Biologically active compounds” or “bioactive compounds” generally refer to chemical compounds having known or suspected biological activity in a mammal. Chemical compounds, as used in this context, can be any atom or molecule having biological activity, e.g., therapeutic activity, in a mammal (e.g., a human). Examples of biologically active compounds include atoms, small molecules, macrocycles, peptides, proteins, antibodies, antigen-binding fragments of antibodies, or nucleic acid molecules (e.g., DNA or RNA). Included in “biologically active molecules” are all cations, anions, salts, oxides, solvates, stereoisomers, and isotopes having known or suspected biological activity. Some examples of biologically active compounds include nucleic acid molecules (e.g., mRNA), radiotherapeutics (e.g., a radioisotope or a chelation complex comprising a radioisotope), drugs (e.g., antiproliferative agents, antineoplastics, cytotoxic or cytostatic agents, targeted therapeutics, anti-inflammatory drugs, a nucleic acid molecule, a peptide, etc.). As disclosed herein, some examples of bioactive molecule for use in the present invention include nucleic acid molecules. Examples of nucleic acid molecules include mRNA, tRNA, miRNA, siRNA, ssDNA, dsDNA, cDNA, genomic DNA, or fragments thereof. As used herein, biologically active compounds or bioactive compounds may refer to a payload (e.g., a therapeutic payload) disclosed herein.

“Payload” as used herein generally refers to a molecule having utility within the interior of a targeted cell. A payload may be useful in that it can be detected or manipulated within a cell. A payload can also be a biologically active molecule or bioactive molecule. In such cases, the payload may exert biological effects (e.g., disease-modifying effects) within a targeted cell. Nucleic acid molecules such as plasmid DNA, mRNA, and siRNA, may be particularly useful as payloads. A lipid nanoparticle comprising a compound disclosed herein and a payload that is a nucleic acid molecule may be particularly useful as a transfection reagent, i.e., for introducing nucleic acids into eukaryotic cells. A payload may also comprise a plurality of different classes of molecules. For example, a payload may refer to a combination of nucleic acid molecules and one or more additional non-nucleic acid molecules (e.g., small molecule therapeutics, chelators, binding agents, etc.). A payload may also comprise a detectable agent or detectable group (e.g., a metal, radiotracer, dye, etc.), a therapeutic agent (e.g., an immunomodulator, anti-cancer drug, antiviral drug, etc.), an oligonucleotide (e.g., siRNA, mRNA), etc.

“Therapeutic agent” or “therapeutic” or “therapeutic payload” generally refer to any disease-modifying or pathogen-directed agent. Therapeutics include any drugs clinically approved for the treatment of diseases. Therapeutics also include small molecules, antibodies, peptides, proteins, radionuclides, radiopharmaceuticals, and the like. Therapeutics are also intended to include oligonucleotides (e.g., siRNA or antisense oligonucleotides). Therapeutic oligonucleotides such as fomivirsen, pegaptanib, mipomersen, defibrotide, eteplirsen, nusinersen, inotersen, patisiran, volanesorsen, givosiran, golodirsen, viltolarsen, lumasiran, inclisiran, and casimersen, are examples of therapeutic agents that can be used in combination with the LNPs and PLips instantly disclosed.

“Nucleic acid” or “nucleic acid molecule” as used herein generally refers to any biopolymer comprising nucleotides (e.g., cytosine, guanine, adenine, uracil, or thymine). In some embodiments, nucleic acids comprise natural nucleotides. Nucleic acids can comprise unnatural nucleotides. In some embodiments, a nucleic acid comprises deoxyribonucleic acid (DNA), ribonucleic acid (RNA), locked nucleic acid (LNA), peptide nucleic acid (PNA), or any combination thereof. In some embodiments, a nucleic acid comprises deoxyribonucleic acid (DNA). In some embodiments, a nucleic acid comprises ribonucleic acid (RNA). In some embodiments, a nucleic acid comprises locked nucleic acid (LNA). In some embodiments, a nucleic acid comprises peptide nucleic acid (PNA). In some embodiments, DNA is single-stranded DNA (ssDNA). In some embodiments, the DNA is double-stranded DNA (dsDNA). In some embodiments, the DNA is circular DNA (cDNA), e.g., plasmid DNA. In some embodiments, the DNA is recombinant DNA (rDNA). In some embodiments, the DNA is genomic DNA. In some embodiments is synthetic DNA, modified DNA, or unnatural DNA. In some embodiments, the RNA is messenger RNA (mRNA). In some embodiments, the RNA is transfer RNA (tRNA). In some embodiments, the RNA is ribosomal RNA (rRNA). In some embodiments, the RNA is small nuclear RNA (snRNA). In some embodiments, the RNA is micro RNA (miRNA). In some embodiments, the RNA is silencing RNA (siRNA). In some embodiments, the RNA is a naked RNA (e.g., a non-enveloped RNA that is not complexed with lipids, proteins, or other stabilizing or protective molecules). In some embodiments, the RNA is complexed RNA.

An “effective amount” or “therapeutically effective amount,” as used herein, generally refers to a sufficient amount of an agent or a compound being administered (e.g., a payload or bioactive molecule), which will relieve to some extent one or more of the symptoms of the diseases or conditions being treated. The result includes reduction or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

“Dispersity” or “Polydispersity index” generally refers to a measure of the broadness of a molecular weight distribution of a polymer, defined as M_{w}/Mₙ, where M_{w} is the weight average molecular weight (alternately, the “mass average molar mass”), and Mₙ is the number average molecular weight (alternatively, the “number-average molar mass”). This value corresponds to the heterogeneity of sizes of molecules in a mixture. In a mixture where all molecules have the same size, the dispersity is 1. A lower dispersity can indicate greater homogeneity within a sample.

A “steric stabilizer” generally refers to a long chain hydrophilic group that prevents aggregation of final polymer by sterically hindering particle-particle electrostatic interactions. Examples include, but are not limited to, alkyl groups, PEG chains, polysaccharides, and alkyl amines. Electrostatic interactions are the non-covalent association of two or more substances due to attractive forces between positive and negative charges.

“Reactive groups” generally refers to chemical moieties capable of forming either an ionic or a covalent bond with another compound, particularly in a biological context (e.g., at approximately neutral pH and approximately 37° C.). The portions of reactive compounds that are capable of forming covalent bonds may be referred to as reactive functional groups. Reactive groups include coupling partners for a particular reaction (e.g., an alkyne or an azide for a “click”-type chemistry or reaction), as well as generally reactive species (e.g., a nucleophile or an electrophile). In some embodiments, a reactive group is a maleimide or a succinimide. In some embodiments, a reactive group is a chemical group capable of performing a bioorthogonal reaction. In some embodiments, the biorthogonal reaction is a copper-free click reaction (CuAAC). In some embodiments, a reactive group is an azide. In some embodiments, a reactive group is an alkyne. In some embodiments, the alkyne is a strained alkyne (e.g., a cycloalkyne). In some embodiments, the cycloalkyne is a monofluorinated cyclooctyne (MOFO), difluorinated cyclooctyne (DIFO), dibenzocyclooctyne (DIBO), dibenzoazacyclooctyne (DIBAC), biarylazacyclooctynone (BARAC), dimethoxyazacyclooctyne (DIMAC), and the like.

“Detectable group” generally refers to a compound or chemical moiety that is capable of being detected by any method known in the art. In some embodiments, a detectable group is capable of bioorthogonal detection. In some embodiments, a detectable group comprises a conjugated aromatic or heteroaromatic system that is capable of emitting light in response to a stimulus (e.g., an excitation). In some embodiments, a detectable group comprises a fluorescent group (e.g., a fluorescein group, or a derivative thereof, a rhodamine group, or a derivative thereof, or a coumarin group, or a derivative thereof). In some embodiments, a detectable group comprises a pyrene group, a benzopyrene group, or a derivative thereof. Fluorescent groups, alternatively “fluorophores,” are well known in the art, and any known fluorophore is considered within the scope of the present invention. In some embodiments, a fluorophore is a fluorescein, rhodamine, coumarin, cyanine, or a xanthene, or a derivative thereof. In some embodiments, the fluorophore is an Alexa Fluor or DyLight Fluor probe, or a derivative thereof.

A “steroid” or “steroid derivative” generally refers to a sterol or a stanol in which the hydroxyl moiety has been modified (e.g., acylated), or a steroid hormone, or an analog thereof. The modification can include spacer groups, linkers, or reactive groups. As used herein, a steroid can be any naturally occurring or unnatural steroid known in the art (e.g., cholesterol), or a derivative thereof.

As used herein, a “cell” generally refers to a biological cell. In some embodiments, a cell is an animal cell (e.g., a human cell) or a plant cell. In some embodiments, a cell is a particular type of cell, e.g., an immune cell, a blood cell, a cancer cell, a healthy cell, etc.

As used herein, the term “in vivo” may be used to describe an event that takes place in an organism, such as a subject's body. In some embodiments, in vivo refers to an event that takes place in the body of a non-human subject, e.g., a mouse or a rat. In some embodiments, in vivo refers to an event that takes place within a human body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

### Lipid-Polymer Compounds (“PLips” or Alternatively, “Compounds”)

In one aspect, disclosed herein are polymer-lipid compounds comprising a lipid, a polymer, and a functional group. A lipid may be bonded to a polymer via a backbone. Lipids may also be bonded to a polymer backbone via a (non-side-chain linker). A polymer may comprise a backbone and side-chains (e.g., a C₁₋₂₀ heteroalkyl side-chain). Alternatively, a polymer may have no side-chain. In some instances, polymers comprising heteroalkyl side-chains interact with a nucleic acid molecule or molecules, thereby stabilizing or encapsulating the nucleic acid molecules. Polymers comprising heteroalkyl side-chains may provide advantageous biological or physicochemical properties compared to unsubstituted (e.g., PEG or PEI) polymers.

In another aspect, disclosed herein is a compound having a structure represented by Formula I:

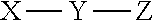

or a pharmaceutically acceptable salt thereof; wherein:
X is a lipid;
Y is a polymer comprising at least 3 monomeric units, each said monomeric unit comprising a C₁₋₂₀ heteroalkyl side-chain; and
Z is an unsubstituted or substituted functional group;
wherein said lipid is covalently bonded to said polymer via a backbone.

### Lipids (“X”)

The “lipid” component(s) of a polymer-lipid compound described herein generally refers to an organic compound (or a radical thereof, in the case of a polymer-lipid compound) that is or contains a lipophilic and/or hydrophobic moiety. In many instances, lipids are poor aqueous solubility, but are soluble in nonpolar solvents. The term lipid as used herein may refer to either a lipophilic group itself, or a derivative thereof. A lipid may further comprise a linker or a spacer. For example, a lipid may comprise a hydrophobic moiety with an optionally substituted alkyl or an optionally substituted heteroalkyl linker, which links the hydrophobic moiety to the polymer (Y). In some instances, a lipid is or comprises a fatty acid. In some instances, a lipid comprises one or more fatty acids or derivatives thereof, and a glyceride group. A lipid may comprise, for example, a monoglyceride, a diglyceride, or triglyceride. These may alternately be referred to as (mono)acyl, diacyl, or triacyl glycerides. As described herein, a lipid may be (or may comprise) an acyl- or diacylglyceride, which is bonded to a polymer backbone.

Lipid can also refer to nonpolar groups that do not comprise a fatty acid. For example, a sterol or a phospholipid, or a derivative thereof, may be referred to as a lipid. Lipids described herein may comprise a linker, which links the monoglyceride, diglyceride, sterol, etc. to the polymer backbone. A linker may be a C₁₋₆ alkyl group or a 1- to 6-membered heteroalkyl group, each being optionally further substituted. In some instances, a linker is a substituted or unsubstituted C₁₋₆ alkyl group, which forms an ester bond with the hydroxy group of a glycerol or sterol. If substituted, a linker group may be substituted, for example, with one or more methyl or cyano groups. In some instances, a linker may have a geminal dimethyl substitution or a geminal methyl/cyano substitution. In some examples, a linker is a substituted or unsubstituted C₁₋₆ alkyl ester. A linker can also be selected from the class of C₁₋₆ ethers, C₁₋₆ amides, C₁₋₆ carbamates, C₁₋₆ phosphoesters, C₁₋₆ sulfonamides, and the like. Alternatively, a longer linker may be employed, comprising about 7 to about 20 carbon atoms, conjugated via an ester linkage and optionally substituted as described above.

A lipid as used herein may refer to any steroid (e.g., a sterol). For example, in some examples, a lipid comprises a sterol or a stanol. Examples of sterol include, but are not limited to, cholesterol, campesterol, stigmasterol, brassicasterol, avenasterol, sitosterol, and ergosterol.

A lipid may comprise one or more fatty acids. For example, a lipid can be diacylglyceride comprising one or more of a saturated fatty acid, a monounsaturated fatty acid, a polyunsaturated fatty acid, or a combination thereof. A fatty acid generally contains an acid group, and a hydrocarbon chain (alternatively, a fatty acid tail). Fatty acid tails may range between about 10 and about 30 carbons in length, and may have between about 0 and about 6 degrees of unsaturation. As used herein, a fatty acid tail may be a C₁₀₋₃₀ alkyl, C₁₀₋₃₀ alkenyl, or C₁₀₋₃₀ alkynyl group. A fatty acid tail may be substituted or unsubstituted. In some cases, a fatty acid tail is an unsubstituted C₁₀₋₃₀ alkyl or C₁₀₋₃₀ alkenyl group. In some cases, a fatty acid tail is a linear or unbranched C₁₀₋₃₀ alkyl or C₁₀₋₃₀ alkenyl group.

In one example, a lipid comprises a monoacylglycerol (MAG) or diacylglycerol (DAG) group having one or more linoleic acid or oleic acid tail(s). The lipid may further comprise a geminally di-substituted C₁₋₆ alkyl ester linker, which links the MAG or DAG to the polymer backbone. In another example, a lipid comprises a cholesterol group. The cholesterol lipid may further comprise a geminally di-substituted C₁₋₆ alkyl ester linker, which links the sterol to the polymer backbone.

As an example, a lipid may have a structure represented by any one of the following formulae:

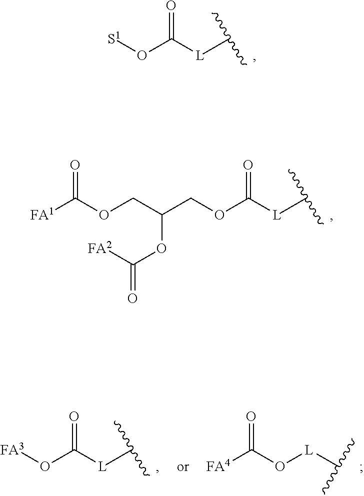

where each L is a substituted or unsubstituted C₁₋₆ alkyl group, S¹ is a sterol group, and each of FA¹, FA², FA³, and FA⁴ is a fatty acid tail having 10 to 30 carbon atoms and 0-3 double bonds per fatty acid tail. In some embodiments, each L is a substituted or unsubstituted C₁₋₁₂ alkyl group, or a substituted or unsubstituted 2- to 12-membered heteroalkyl group. As described herein, L may comprise a geminal substitution (e.g., dimethyl or methyl/cyano). In other instances, a linker L is mono-substituted. In still others, the linker L is unsubstituted. Examples of linkers include, but are not limited to:

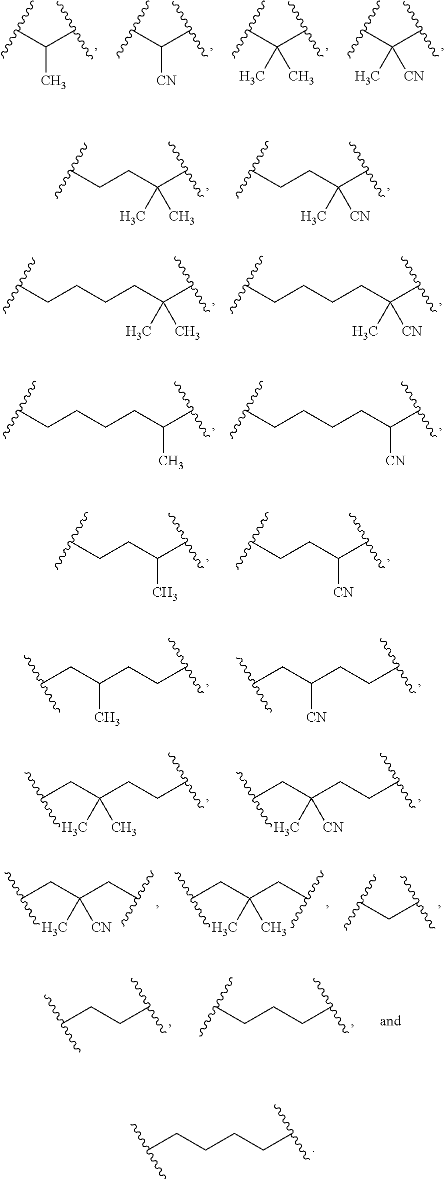

In further examples, a lipid may have a structure represented by any one of the following formulae:

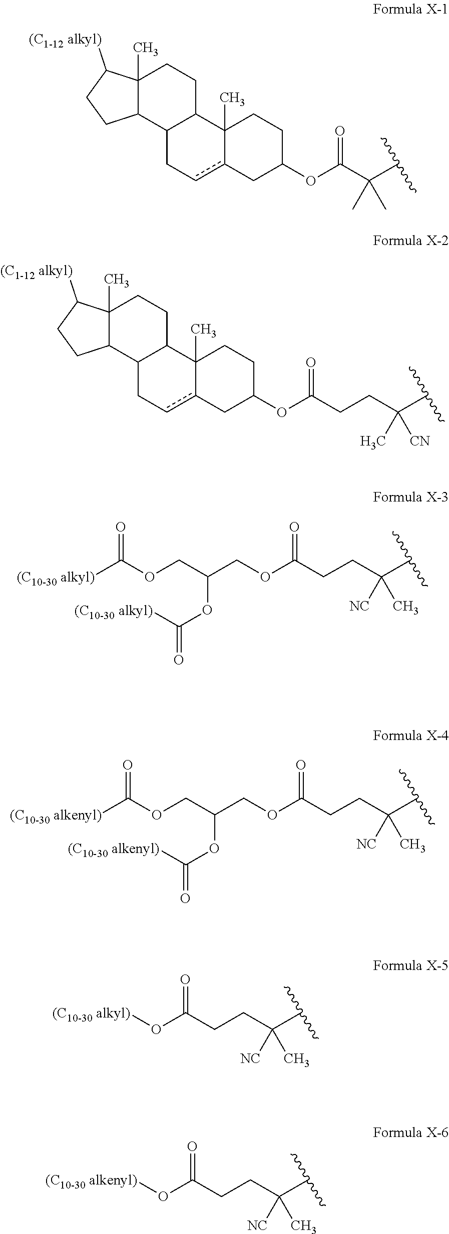

As used herein, alkenyl may include dienes and trienes, and further includes both cis (or E) and trans (or Z) isomers. Stereoisomers (e.g., enantiomers, diastereomers, etc.) of all forms are also considered within the scope of the formulae above, and throughout this disclosure, unless otherwise noted.

A lipid may be hydrophobic. In some examples, a lipid is amphiphilic. In some embodiments, the lipid comprises an octanol:water coefficient (log(K_{OW})) of at least 2, at least 3, at least 4, at least 5, at least 6, at least 7, at least 8, at least 9, or at least 10. In some instances, a lipid comprises log(K_{OW}) ranges from about 2 to about 10. In further instances, a lipid comprises log(K_{OW}) ranges from about 2 to about 3, from about 2 to about 4, from about 2 to about 5, from about 2 to about 6, from about 2 to about 7, from about 2 to about 8, from about 2 to about 9, from about 2 to about 10, from about 3 to about 4, from about 3 to about 5, from about 3 to about 6, from about 3 to about 7, from about 3 to about 8, from about 3 to about 9, from about 3 to about 10, from about 4 to about 5, from about 4 to about 6, from about 4 to about 7, from about 4 to about 8, from about 4 to about 9, from about 4 to about 10, from about 5 to about 6, from about 5 to about 7, from about 5 to about 8, from about 5 to about 9, from about 5 to about 10, from about 6 to about 7, from about 6 to about 8, from about 6 to about 9, from about 6 to about 10, from about 7 to about 8, from about 7 to about 9, from about 7 to about 10, from about 8 to about 9, from about 8 to about 10, or from about 9 to about 10. A lipid may have a log(K_{OW}) range of about 2, about 3, about 4, about 5, about 6, about 7, about 8, about 9, or about 10. A lipid may have a log(K_{OW}) of at least about 2, about 3, about 4, about 5, about 6, about 7, about 8, or about 9. A lipid may have a log(K_{OW}) of at most about 3, about 4, about 5, about 6, about 7, about 8, about 9, or about 10.

A compound disclosed herein may have a lipid of the formula:

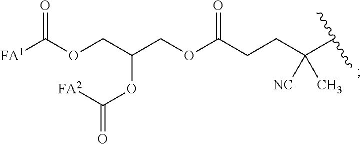

wherein each of FA¹ and FA² is independently an ester side-chain derived from one of the following fatty acids:

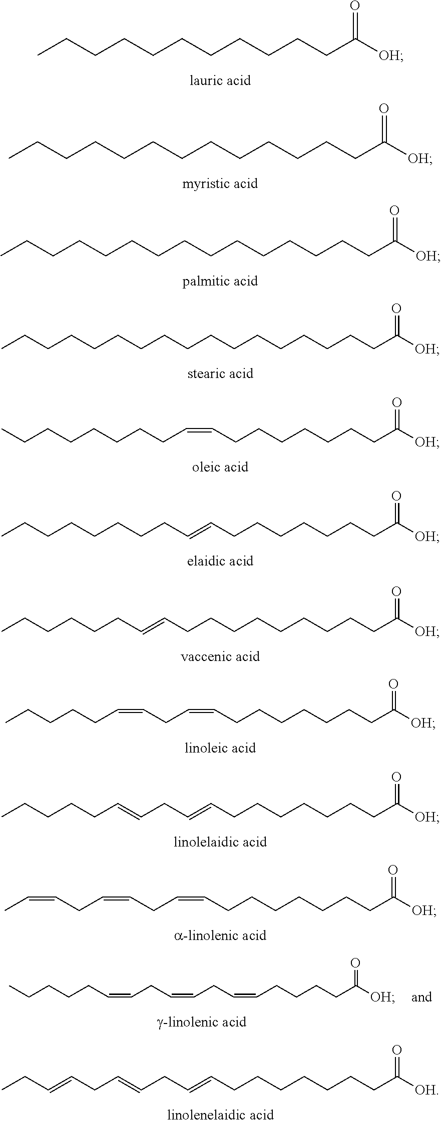

In additional examples, a compound disclosed herein may have a lipid of the formula:

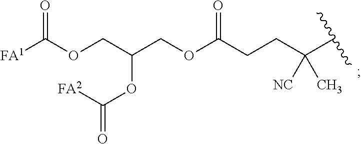

wherein each of FA¹ and FA² is independently selected from the group consisting of C₁₋₃₀ alkyl, C₁₋₃₀ alkenyl, C₁₋₃₀ alkynyl, or 1- to 30-membered heteroalkyl. Some examples of 1- to 30

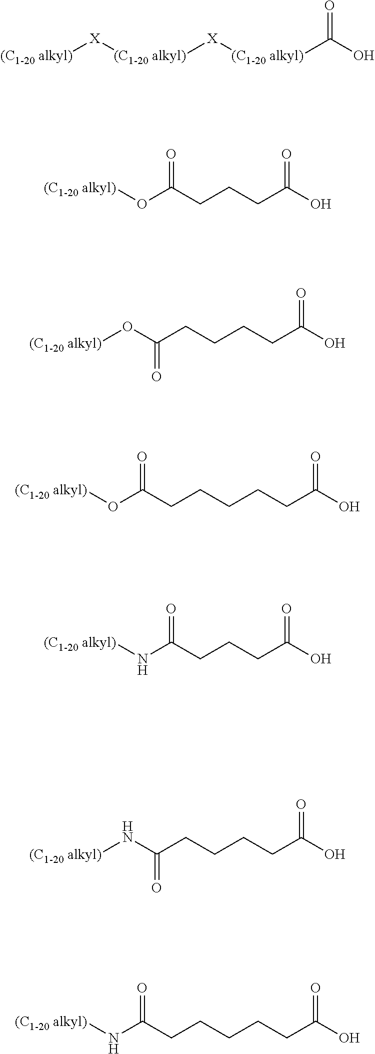

A compound disclosed herein may have a lipid of the formula:

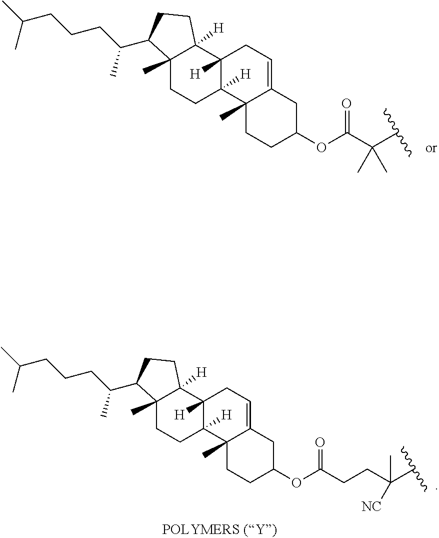

In the literature, PEG-DMG is a ubiquitously utilized stabilizing lipid in LNPs. Several studies and clinical reports suggest that moderate to severe immunogenic responses can occur after systemic PEG administration. Acute hypersensitivity and a decreasing therapeutic efficiency of PEGylated drugs can result from complement system activation and/or anti-PEG antibody production. Various stabilizing PLips have been synthesized and shown to form stable LNPs of similar sizes compared to LNPs stabilized with PEG-DMG. A stabilizing PLip can contain any variation of lipid tail (cholesterol, diacyl, acyl, etc.), while the polymer side-chain(s) can comprise a hydrophilic, stabilizing group. Stabilizing polymer side-chains may be referred to as heteroalkyl side-chains, stabilizing side-chains, non-cationic side-chains, or solubilizing side-chains. Some examples of the stabilizing PLip side-chains include repeat units of 2-hydroxypropylmethacrylamide (HPMA), (2-(methylsulfinyl)ethyl methacrylate (MSEMA), hydroxyethylacrylate (HEA), and oligo(ethylene glycol) methacrylate. The size and stability of the LNPs may depend on the nature and length of the polymer side-chain. In addition, size and stability of the LNP may be determined or affected by the identity of the lipid tail present in the stabilizing PLip. In some instances, a stabilizing PLip is used as a replacement for a PEG stabilizing agent (e.g., PEG-DMG). Alternatively, a PLip may have both stabilizing and cationic properties. For example, a PLip may comprise multiple polymer blocks (sequential, alternating, statistical, random), where one block (or plurality of identical monomeric units) contains a cationic or cation-forming group, whereas a second block (or plurality of identical monomeric units) contains a stabilizing side-chain. Stabilizing PLips may comprise a polymer having an alkoxy or hydroxy side-chain. In some cases, the stabilizing PLip is hydrophilic. A stabilizing PLip may contain between about 3 and about 200 stabilizing monomeric units. A cationic monomeric unit may have the structure of one of the following formulae:

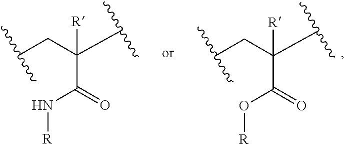

wherein each R group is a C₁₋₆ alkyl, C₁₋₆ alkoxyalkyl, C₁₋₆ hydroxyalkyl, or wherein R is a polyethylene glycol (PEG) group containing 1 to 100 PEG units; and each R′ group is hydrogen or methyl.

Some examples of stabilizing monomeric units include, but are not limited to the following:

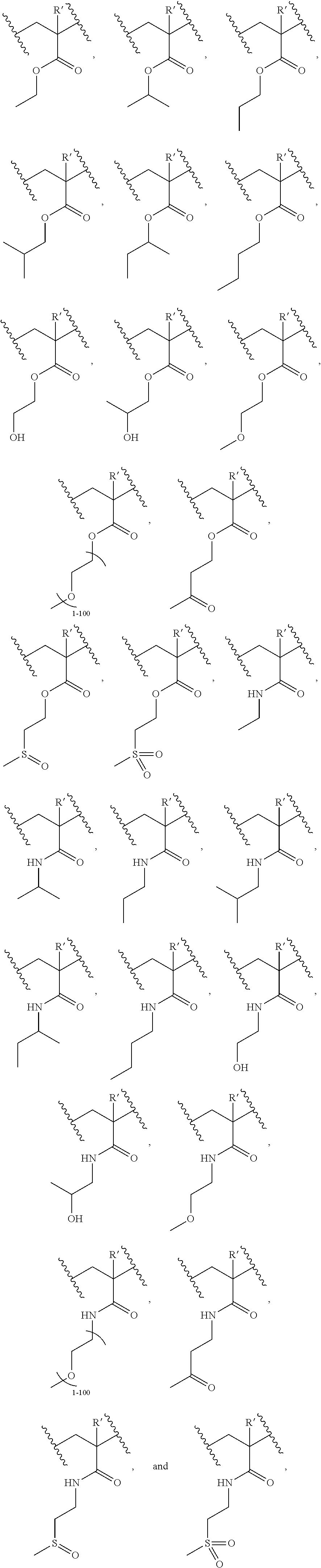

wherein each R′ is hydrogen or methyl. More specifically, a stabilizing monomeric unit may have a structure of one of the following formulae:

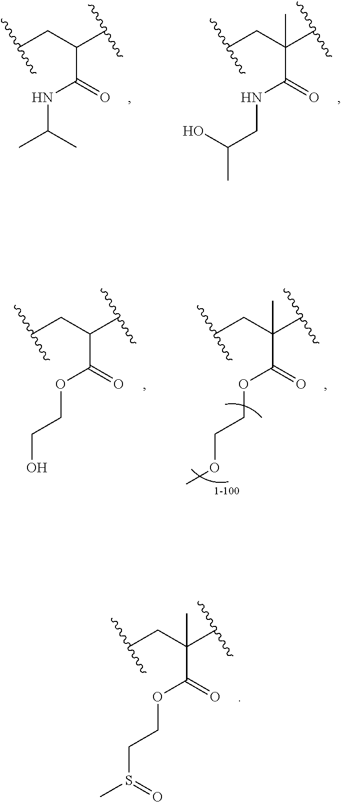

A cationic PLip can contain any variation of a lipid tail (cholesterol, diacyl, acyl, etc.), while the polymer side-chain(s) can comprise a cationic or cation-forming group (e.g., an amine). For example, a cationic PLip can comprise a cationic polymer, wherein at least one (e.g., at least 3) monomeric units of the polymer comprise a cationic or cation-forming side-chain. As used herein, cationic is intended to not only include cations, but also uncharged groups that become cationic in biological environments. For example, a cationic side-chain can comprise an amide or ester group, wherein the amide or ester is substituted with an alkylamine or alkylammonium side-chain. A cationic PLip may contain between about 3 and about 200 cationic monomeric units. A cationic monomeric unit may have the structure of one of the following formulae:

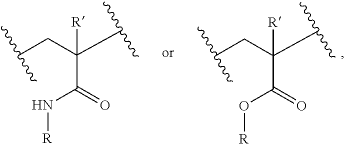

wherein each R group is an alkylamine or alkylammonium, and wherein each R′ group is hydrogen or methyl. More specifically, a cationic monomeric unit may have the structure of one of the following formulae:

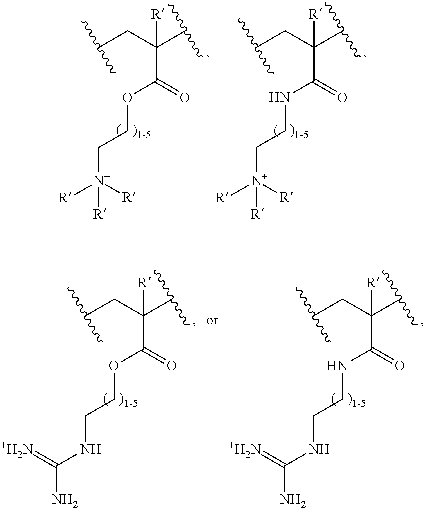

or a salt thereof, wherein each R′ is independently hydrogen or methyl.

Some examples of cationic monomeric units include, but are not limited to the following:

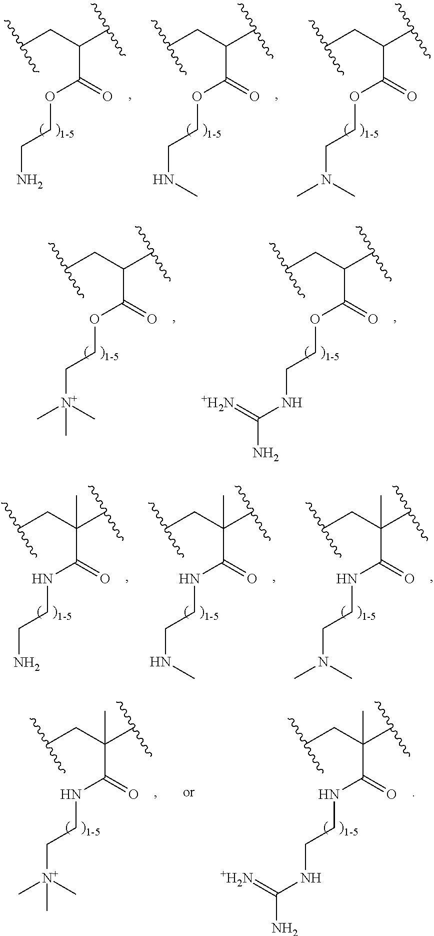

In each of the preceding monomeric unit examples, the alkyl chain can be between 2 and 6 carbons in length (e.g., 2, 3, or 4 carbons in length). For example, in any one of the preceding monomeric units, the alkyl chain may be an ethyl chain, a propyl chain, or a butyl chain. In specific embodiments the cationic monomeric unit is

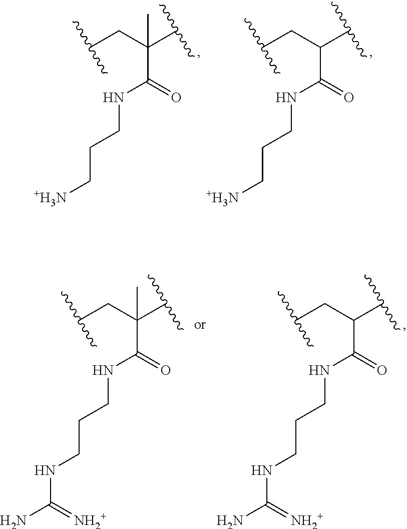 [SMILES: *CC(*)(C)C(=O)NCCC[NH3+] ; *CC(*)C(=O)NCCC[NH3+] ; *CC(*)(C)C(=O)NCCCNC(N)=[NH2+] ; *CC(*)C(=O)NCCCNC(N)=[NH2+]]

or a salt thereof, or a free base thereof.

In some examples, a polymer (e.g., a polymer having a structure represented by Formula Y-A, Formula Y-B, Formula Y-C, or Formula Y-D) comprises at least a first plurality of monomeric units represented by one of the following formula:

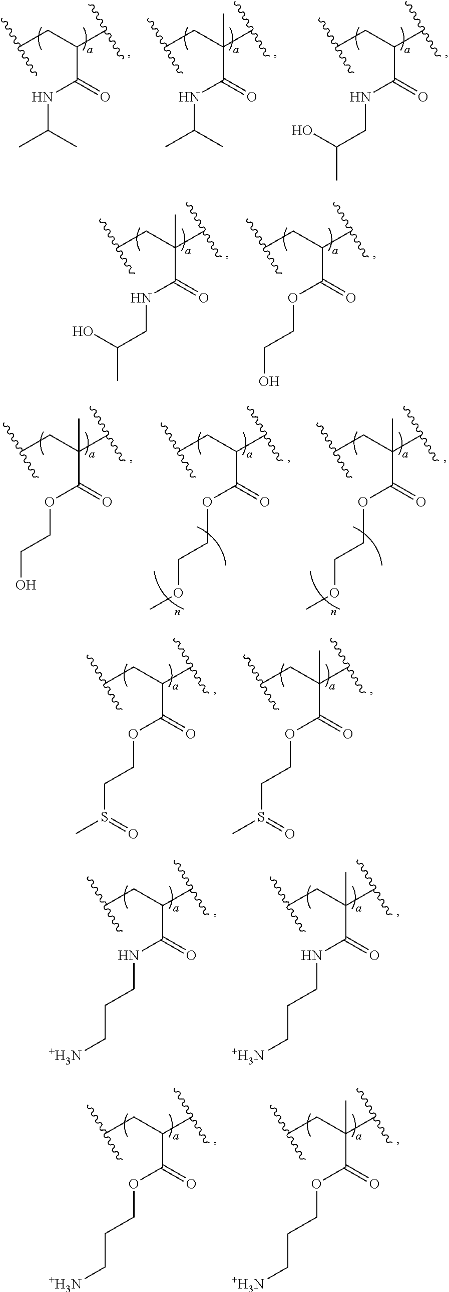

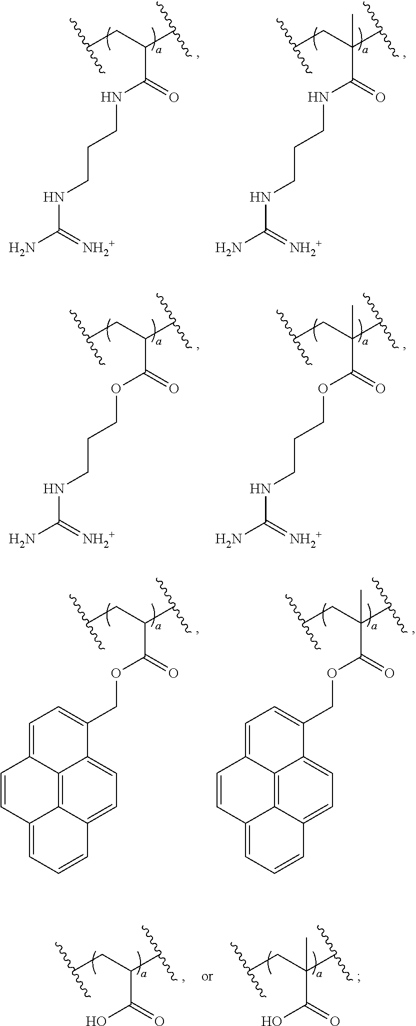

wherein a is an integer from 3 to 400, and where n is an integer from 1 to 100.

More specifically, n can be an integer from 1 to about 50. In more specific embodiments, n can be an integer from 1 to 40, 1 to 30, 1 to 20, 1 to 10, 2 to 10, or 2 to 8. In certain examples disclosed herein, n has an average value between 2 and 10, between 3 and 6, or between 4 and 5 (e.g., n is about 4.5). N can be an integer having a value (on average) that is about 2, about 3, about 4, about 5, about 6, about 7, about 8, about 9, or about 10.

In another aspect, provided herein are compounds comprising a lipid connected to a backbone of a polymer, wherein the polymer comprises 3 monomeric units or more. In some instances, a monomeric unit comprises a C₁₋₂₀ heteroalkyl side-chain.

A polymer may comprise about 5 monomeric units or more, about 10 monomeric units or more, about 15 monomeric units or more, about 20 monomeric units or more, about 30 monomeric units or more, about 50 monomeric units or more, about 100 monomeric units or more, about 200 monomeric units or more, about 300 monomeric units or more, or about 400 monomeric units or more. Each monomeric unit may comprise a C₁₋₂₀ heteroalkyl side chain.

In some instances, a polymer can comprise about 3 monomeric units to about 400 monomeric units. For example, the polymer may comprise about 3 monomeric units to about 4 monomeric units, about 3 monomeric units to about 10 monomeric units, about 3 monomeric units to about 30 monomeric units, about 3 monomeric units to about 50 monomeric units, about 3 monomeric units to about 80 monomeric units, about 3 monomeric units to about 100 monomeric units, about 3 monomeric units to about 120 monomeric units, about 3 monomeric units to about 150 monomeric units, about 3 monomeric units to about 200 monomeric units, about 3 monomeric units to about 400 monomeric units, about 4 monomeric units to about 10 monomeric units, about 4 monomeric units to about 30 monomeric units, about 4 monomeric units to about 50 monomeric units, about 4 monomeric units to about 80 monomeric units, about 4 monomeric units to about 100 monomeric units, about 4 monomeric units to about 120 monomeric units, about 4 monomeric units to about 150 monomeric units, about 4 monomeric units to about 200 monomeric units, about 4 monomeric units to about 400 monomeric units, about 10 monomeric units to about 30 monomeric units, about 10 monomeric units to about 50 monomeric units, about 10 monomeric units to about 80 monomeric units, about 10 monomeric units to about 100 monomeric units, about 10 monomeric units to about 120 monomeric units, about 10 monomeric units to about 150 monomeric units, about 10 monomeric units to about 200 monomeric units, about 10 monomeric units to about 400 monomeric units, about 30 monomeric units to about 50 monomeric units, about 30 monomeric units to about 80 monomeric units, about 30 monomeric units to about 100 monomeric units, about 30 monomeric units to about 120 monomeric units, about 30 monomeric units to about 150 monomeric units, about 30 monomeric units to about 200 monomeric units, about 30 monomeric units to about 400 monomeric units, about 50 monomeric units to about 80 monomeric units, about 50 monomeric units to about 100 monomeric units, about 50 monomeric units to about 120 monomeric units, about 50 monomeric units to about 150 monomeric units, about 50 monomeric units to about 200 monomeric units, about 50 monomeric units to about 400 monomeric units, about 80 monomeric units to about 100 monomeric units, about 80 monomeric units to about 120 monomeric units, about 80 monomeric units to about 150 monomeric units, about 80 monomeric units to about 200 monomeric units, about 80 monomeric units to about 400 monomeric units, about 100 monomeric units to about 120 monomeric units, about 100 monomeric units to about 150 monomeric units, about 100 monomeric units to about 200 monomeric units, about 100 monomeric units to about 400 monomeric units, about 120 monomeric units to about 150 monomeric units, about 120 monomeric units to about 200 monomeric units, about 120 monomeric units to about 400 monomeric units, about 150 monomeric units to about 200 monomeric units, about 150 monomeric units to about 400 monomeric units, or about 200 monomeric units to about 400 monomeric units. In some embodiments, the polymer comprises about 3 monomeric units, about 4 monomeric units, about 10 monomeric units, about 30 monomeric units, about 50 monomeric units, about 80 monomeric units, about 100 monomeric units, about 120 monomeric units, about 150 monomeric units, about 200 monomeric units, or about 400 monomeric units. In some embodiments, the polymer comprises at least about 3 monomeric units, about 4 monomeric units, about 10 monomeric units, about 30 monomeric units, about 50 monomeric units, about 80 monomeric units, about 100 monomeric units, about 120 monomeric units, about 150 monomeric units, or about 200 monomeric units. In some embodiments, the polymer comprises at most about 4 monomeric units, about 10 monomeric units, about 30 monomeric units, about 50 monomeric units, about 80 monomeric units, about 100 monomeric units, about 120 monomeric units, about 150 monomeric units, about 200 monomeric units, or about 400 monomeric units. Each monomeric unit comprises a C₁₋₂₀ heteroalkyl side chain.

A polymer may be a polyacrylate, also called, a poly(acrylic ester) or a poly(acrylic acid alkyl ester). A polyacrylate polymer has one or more monomeric units comprising an acrylate, also called, an acrylic ester, or an acrylic acid alkyl ester. A polyacrylate polymer may have about 10 or more monomeric units comprising an acrylate. In some instances, a polymer may have up to about 400 monomeric units comprising an acrylate. For example, a polymer may have about 10 to about 400 monomeric units comprising an acrylate. In other examples, a polymer can have from about 20 to about 30), from about 50 to about 200, from about 100 to about 150.

In some embodiments, the polymer is a polyacrylamide. In some embodiments, the polymer has a monomeric unit comprising an acrylamide, also called, acrylic amide. In some embodiments, the polymer comprises a combination of acrylates and acrylamides.

In some embodiments, the polymer comprises a series of repeating units, wherein the repeating units comprise two monomers, three monomers, or four monomers. In some embodiments, the repeating units comprise both an acrylamide and an acrylate. In some embodiments, the repeating units comprise two or more acrylamides. In some embodiments, the repeating units comprise two or more acrylates.

In some embodiments, the polymer is a homopolymer comprising an acrylate. In some embodiments, the homopolymer comprises a cationic monomeric unit. In some embodiments, the homopolymer is positively charged in neutral aqueous solution.

In some embodiments, the polymer is a homopolymer comprising an acrylamide. In some embodiments, the homopolymer comprises a cationic monomeric unit. In some embodiments, the homopolymer is positively charged in neutral aqueous solution.

In some embodiments, the polymer is a copolymer comprising a block copolymer, an alternating copolymer, a random or statistical copolymer, or a gradient copolymer. In some embodiments, the copolymer comprises a cationic monomeric unit. In some embodiments, the polymer is positively charged in neutral aqueous solution.

In some embodiments, the polymer is a cationic polymer, e.g., comprising a plurality of cationic or cation-forming groups (e.g., primary amines). In some embodiments, the cationic polymer comprises a plurality of monomeric units, wherein each monomeric unit comprises an aminoalkyl side-chain (e.g., CH₂CH₂NH₂, CH₂CH₂CH₂NH₂, or a cation thereof). In some embodiments, the cationic polymer comprises a plurality of monomeric units, wherein each monomeric unit comprises an alkylammonium side-chain (e.g., CH₂CH₂N(CH₃)₃⁺, CH₂CH₂CH₂N(CH₃)₃⁺, or a salt thereof). In some embodiments, a cationic PLip comprises at least a first plurality of monomeric units, wherein each said monomeric unit of said first plurality of monomeric units comprises an aminoalkyl (or alkylamino) side-chain (e.g., comprises an —NH₂ or —N(CH₃)₃⁺ group). In some embodiments, the cationic PLip comprises a second plurality of monomeric units, wherein each said monomeric unit of said second plurality of monomeric units comprises a hydroxyalkyl or alkoxyalkyl side-chain.

In some embodiments, the polymer comprises a pK_{b} ranging from about 2 to about 12.

In some embodiments, the polymer comprises a pK_{b} ranging from about 2 to about 3, from about 2 to about 4, from about 2 to about 5, from about 2 to about 6, from about 2 to about 7, from about 2 to about 8, from about 2 to about 9, from about 2 to about 10, from about 2 to about 11, from about 2 to about 12, from about 3 to about 4, from about 3 to about 5, from about 3 to about 6, from about 3 to about 7, from about 3 to about 8, from about 3 to about 9, from about 3 to about 10, from about 3 to about 11, from about 3 to about 12, from about 4 to about 5, from about 4 to about 6, from about 4 to about 7, from about 4 to about 8, from about 4 to about 9, from about 4 to about 10, from about 4 to about 11, from about 4 to about 12, from about 5 to about 6, from about 5 to about 7, from about 5 to about 8, from about 5 to about 9, from about 5 to about 10, from about 5 to about 11, from about 5 to about 12, from about 6 to about 7, from about 6 to about 8, from about 6 to about 9, from about 6 to about 10, from about 6 to about 11, from about 6 to about 12, from about 7 to about 8, from about 7 to about 9, from about 7 to about 10, from about 7 to about 11, from about 7 to about 12, from about 8 to about 9, from about 8 to about 10, from about 8 to about 11, from about 8 to about 12, from about 9 to about 10, from about 9 to about 11, from about 9 to about 12, from about 10 to about 11, from about 10 to about 12, or from about 11 to about 12. In some embodiments, the polymer comprises a pK_{b} ranging from about 2, about 3, about 4, about 5, about 6, about 7, about 8, about 9, about 10, about 11, or about 12. In some embodiments, the polymer comprises a pK_{b} ranging from at least about 2, about 3, about 4, about 5, about 6, about 7, about 8, about 9, about 10, or about 11. In some embodiments, the polymer comprises a pK_{b} ranging from at most about 3, about 4, about 5, about 6, about 7, about 8, about 9, about 10, about 11, or about 12.

A polymer may have a structure represented by Formula Y-A, Formula Y-B, Formula Y-C, or Formula Y-D:

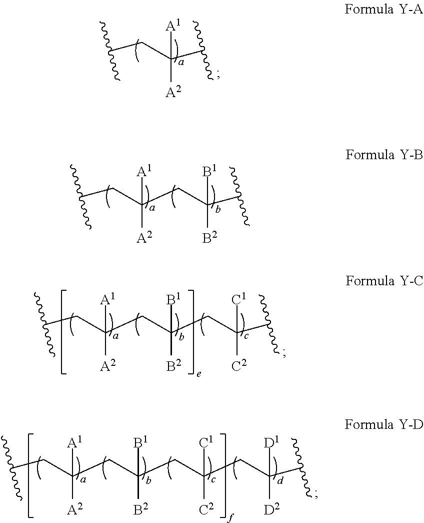

or a pharmaceutically acceptable salt thereof; wherein:
A¹, B¹, C¹, and D¹ are in each instance independently selected from hydrogen and methyl;
A², B², C², and D² are in each instance independently selected from C₁₋₂₀ heteroalkyl;
a, b, c, d, e, and f are in each instance independently an integer selected from 0 to 400 (e.g., 0 to 200 (e.g., 40 to 120)), provided the total number of monomeric units is 3 or more;
wherein if the C₁₋₂₀ heteroalkyl is substituted, then it is substituted with unsubstituted or substituted cycloalkyl, unsubstituted or substituted heterocyclyl, unsubstituted or substituted aryl, unsubstituted or substituted heteroaryl.

In some embodiments, the cycloalkyl is a C₃₋₂₄ cycloalkyl ring. In some embodiments, the cycloalkyl is a C₃₋₁₅ cycloalkyl ring. In some embodiments, the cycloalkyl is a C₃₋₁₀ cycloalkyl ring. In some embodiments, the cycloalkyl is a C₃₋₆ cycloalkyl ring. In some embodiments, the heterocyclyl is a 3- to 24-membered heterocycle. In some embodiments, the heterocyclyl is a 3- to 15-membered heterocycle. In some embodiments, the heterocyclyl is a 3- to 10-membered heterocycle. In some embodiments, the heterocyclyl is a 3- to 6-membered heterocycle. In some embodiments, the heterocyclyl is an oxirane, oxetane, tetrahydrofuran, tetrahydropyran, aziridine, azetidine, pyrrolidine, piperidine, piperazine, morpholine, or dioxane. In some embodiments, the heterocyclyl is a saturated or partially saturated heterocyclic ring consisting of atoms selected from hydrogen, C, N, O, and S.

In some embodiments, the aryl is a C₆₋₂₄ aromatic ring. In some embodiments, the aryl is a C₆₋₁₆ aromatic ring. In some embodiments, the aryl is a C₆₋₁₀ aromatic ring. In some embodiments, the aryl is a phenyl, naphthyl, phenanthrenyl, anthracenyl, fluoroanthenyl, pyrenyl, chrysenyl, benzo[a]pyrenyl, pyrelenyl, or coronenyl. In some embodiments, the aryl is a phenyl, naphthyl, or pyrenyl. In some embodiments, the heteroaryl is a 3- to 24-membered aromatic heterocycle. In some embodiments, the heteroaryl is a 3- to 15-membered aromatic heterocycle. In some embodiments, the heteroaryl is a 3- to 10-membered aromatic heterocycle. In some embodiments, the heteroaryl is a 3- to 6-membered aromatic heterocycle. In some embodiments, the heteroaryl is an aromatic heterocyclic ring consisting of atoms selected from hydrogen, C, N, O, and S.

In some embodiments, the substituted cycloalkyl, substituted heterocyclyl, substituted aryl, or substituted heteroaryl is substituted with one or more groups selected from: oxo, —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CHs, —S(O)₂CH₃, and —S(O)₂OH.

In some embodiments, the polymer has the structure of Formula Y-A:

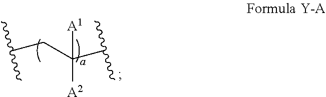

or a pharmaceutically acceptable salt thereof; wherein:
A¹ is hydrogen or methyl;
A² is selected from unsubstituted or substituted C₁₋₂₀ heteroalkyl; and
a is an integer from 3 to 400 (e.g., 3 to 200 (e.g., 40 to 120))

In some embodiments, each A² is the same (i.e., the polymer comprises a single repeating monomer). In some embodiments, each A² is not the same (i.e., the polymer comprises a plurality of different monomers). In some embodiments, A¹ is hydrogen. In some embodiments, A¹ is methyl. In some embodiments, A² is an acid, an ester, a carboxamide, or a substituted amide. In some embodiments, the polymer comprises 3 to 400 monomers (e.g., A1-A12), selected from:

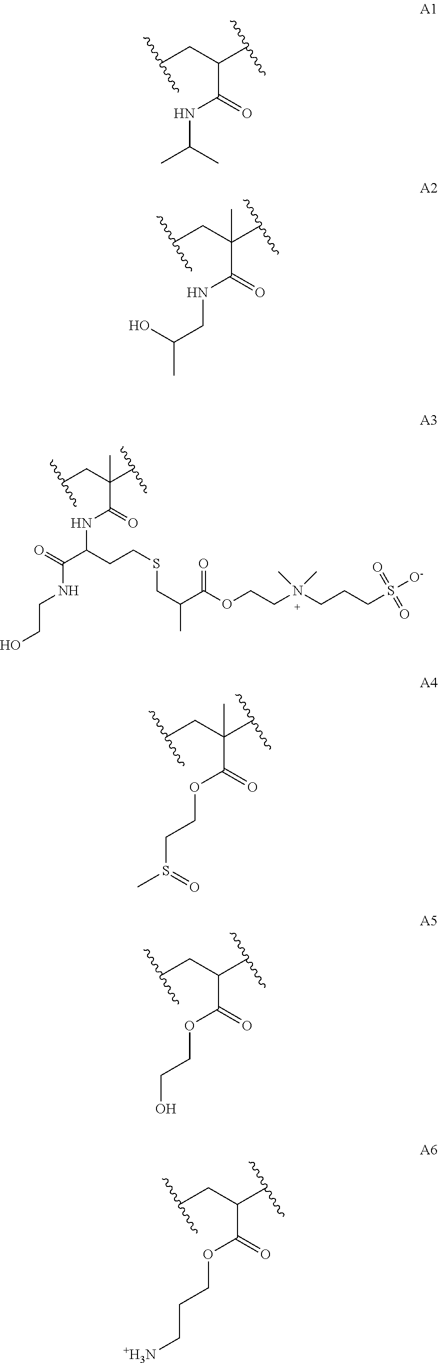

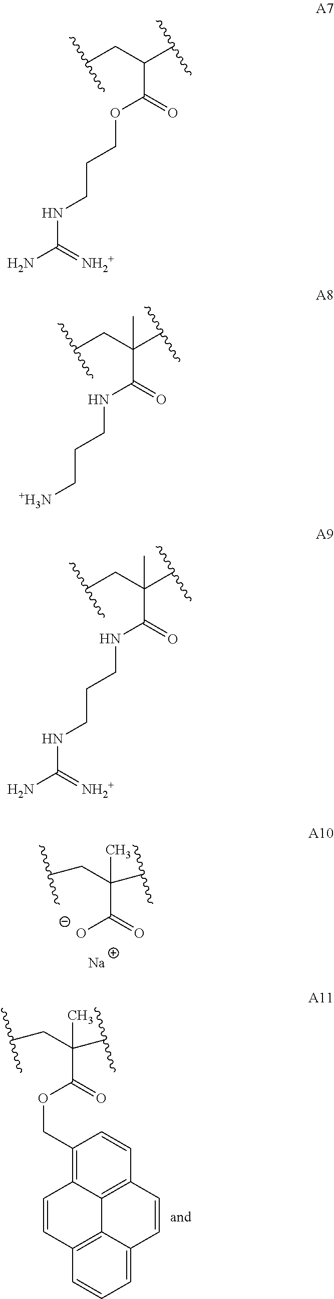

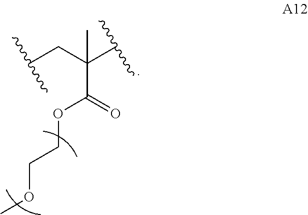

wherein a is an integer from 0 to 100.

In some embodiments, a is an integer from 3 to 400. In some embodiments, a is an integer from 10 to 200. In some embodiments, a is an integer from about 10 to about 30. In some embodiments, a is an integer from about 30 to about 60. In some embodiments, a is an integer from about 60 to about 90. In some embodiments, a is an integer from about 90 to about 120. In some embodiments, a is an integer from about 120 to about 150. In some embodiments, a is an integer from about 40 to about 120. In some embodiments, a is about 10, about 20, about 30, about 40, about 50, about 60, about 70, about 80, about 90, about 100, about 110, about 120, about 130, about 140, about 150, about 200, or more. In some embodiments, a is about 20. In some embodiments, a is about 30. In some embodiments, a is about 40. In some embodiments, a is about 50. In some embodiments, a is about 75. In some embodiments, a is about 95. In some embodiments, a is about 100. In some embodiments, a is about 110. In some embodiments, a is about 115.

In some embodiments, the polymer has the structure of Formula Y-B:

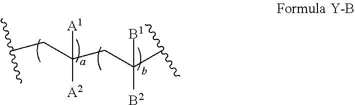

or a pharmaceutically acceptable salt thereof; wherein:
A¹ and B¹ are in each instance independently selected from hydrogen and methyl;
A² and B² are in each instance independently selected from C₁₋₂₀ heteroalkyl; and
a and b are each independently an integer from 0 to 400 (e.g., 0 to 200 (e.g., 40 to 120)), provided the total number of monomeric units is 3 or more.

In some embodiments, each A² is the same, and each B² is the same. In some embodiments, a polymer of Formula Y-B is a polymer comprising two different monomers. In some embodiments, a polymer of Formula Y-B is a polymer comprising two adjacent sequences of repeating monomers (i.e., block copolymers), wherein each A² is the same in each instance, and wherein each B² is the same in each instance, but each B² is not the same as each A². In some embodiments, each A² and B² is a C₁₋₁₀ heteroalkyl. In some embodiments, each A² is an amide, and each B² is an ester. In some embodiments, each A² is an ester, and each B² is an amide. In some embodiments, each A² and B² is a C₁₋₂₀ heteroalkyl group having the formula —C(O)OR² or —C(O)NR⁴R⁵, wherein each R², R⁴, and R⁵ is as defined herein. In some embodiments, each A² and B² is a C₁₋₂₀ heteroalkyl group having the formula —C(O)OR¹² or —C(O)NR¹⁴R¹⁵, wherein each R¹², R¹⁴, and R¹⁵ is as defined herein.

In some embodiments, the polymer of Formula Y-B comprises monomers:

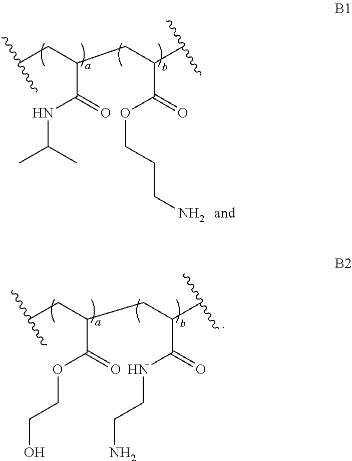

In some embodiments, the polymer has the structure of Formula Y-AB:

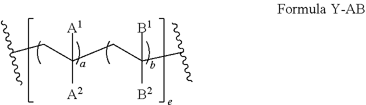

or a pharmaceutically acceptable salt thereof; wherein:
A¹ and B¹ are in each instance independently selected from hydrogen and methyl;
A² and B² are in each instance independently selected from C₁₋₂₀ heteroalkyl;
a, b, and e are in each instance independently an integer from 0 to 400 (e.g., 0 to 200 (e.g., 40 to 120)), provided the total number of monomeric units is 3 or more.

In some embodiments, a polymer of Formula Y-AB is a polymer comprising two adjacent monomers that form a repeating unit, wherein each A² is the same in each instance, and wherein each B² is the same in each instance, but each B² is not the same as each A². In some embodiments, a and b are each 1, and the polymer of Formula Y-AB is an “alternating” copolymer. In some embodiments, a, b, and e are in each instance an integer selected from 0 to 400 (e.g., a random copolymer). In some embodiments, each A² and B² is a C₁₋₁₀ heteroalkyl. In some embodiments, each A² is an amide, and each B² is an ester. In some embodiments, each A² is an ester, and each B² is an amide. In some embodiments, each A² and B² is a C₁₋₂₀ heteroalkyl group having the formula —C(O)OR² or —C(O)NR⁴R⁵, wherein each R², R⁴, and R⁵ is as defined herein. In some embodiments, each A² and B² is a C₁₋₂₀ heteroalkyl group having the formula —C(O)OR¹² or —C(O)NR¹⁴R¹⁵, wherein each R¹², R¹⁴, and R¹⁵ is as defined herein.

In some embodiments, the polymer has the structure of Formula Y-C:

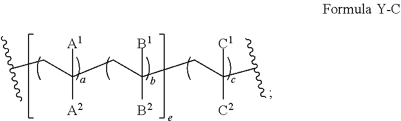

or a pharmaceutically acceptable salt thereof, wherein:
A¹, B¹, and C¹ are in each instance independently selected from hydrogen and methyl;
A², B², and C² are in each instance independently selected from C₁₋₂₀ heteroalkyl:
a, b, c, and e are in each instance independently an integer selected from 0 to 400 (e.g., 0 to 200 (e.g., 40 to 120)), provided the total number of monomeric units is 3 or more.

In some embodiments, a polymer of Formula Y-C is a polymer comprising two adjacent monomers that form a repeating unit, wherein each A² is the same in each instance, and wherein each B² is the same in each instance, but each B² is not the same as each A²; and each C² is a monomer side-chain that can be the same as A², the same as B², or different from both.

In some embodiments, each A², B² and C² is a C₁₋₁₀ heteroalkyl. In some embodiments, each A² is an amide, each B² is an ester, and each C² is an ester. In some embodiments, each A² is an amide, each B² is an ester, and each C² is an amide. In some embodiments, each A² is an ester, each B² is an amide, and each C² is an ester. In some embodiments, each A² is an ester, each B² is an amide, and each C² is an amide. In some embodiments, each A², B², and C² is a C₁₋₂₀ heteroalkyl group having the formula —C(O)OR² or —C(O)NR⁴R⁵, wherein each R², R⁴, and R⁵ is as defined herein. In some embodiments, each A², B², and C² is a C₁₋₂₀ heteroalkyl group having the formula —C(O)OR¹² or —C(O)NR¹⁴R¹⁵, wherein each R¹², R¹⁴, and R¹⁵ is as defined herein.

In some embodiments, the polymer of Formula Y-C is:

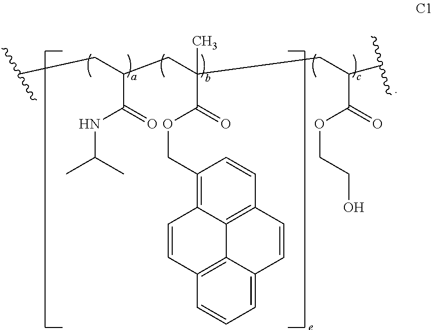

In analogy to Formula Y-AB, in some embodiments, the polymer has a structure of Formula Y-ABC:

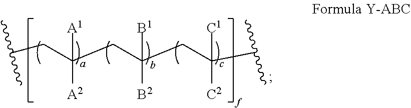

or a pharmaceutically acceptable salt thereof; wherein:
A¹, B¹, and C¹ are in each instance independently selected from hydrogen and methyl;
A², B², and C² are in each instance independently selected from C₁₋₂₀ heteroalkyl; and
a, b, c, and f are in each instance independently an integer selected from 0 to 400 (e.g., 0 to 200 (e.g., 40 to 120)), provided the total number of monomeric units is 3 or more.

In some embodiments, a, b, and c are each 1, and the polymer of Formula Y-ABC is an “alternating” copolymer. In some embodiments, the polymer of Formula Y-ABC contains trimeric repeating units (wherein A, B, and C represent adjacent monomeric units in a given repeating unit). In some embodiments, the polymer of Formula Y-ABC has f repeating units, wherein f is an integer between 0 and 400 (e.g., 0 to 200 (e.g., 40 to 120)), provided the total number of monomeric units is 3 or more. In some embodiments, each A², B² and C² is a C₁₋₁₀ heteroalkyl. In some embodiments, each A², B² and C² is an amide. In some embodiments, each A², B² and C² is an ester. In some embodiments, the polymer has a ratio of about 2:1 of amide side-chains to ester side-chains.

In some embodiments, the polymer has the structure of Formula Y-D:

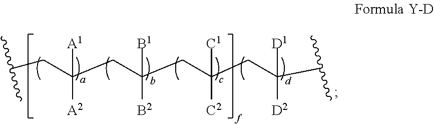

or a pharmaceutically acceptable salt thereof; wherein:
A¹, B¹, C¹, and D¹ are in each instance independently selected from hydrogen and methyl;
A², B², C², and D² are in each instance independently selected from C₁₋₂₀ heteroalkyl; a, b, c, d, e, and f are in each instance independently an integer selected from 0 to 400 (e.g., 0 to 200 (e.g., 40 to 120)), provided the total number of monomeric units is 3 or more.

In some embodiments, a, b, and c are each 1, and the polymer of Formula Y-ABC is a mixed copolymer, comprising a first block having a trimeric repeating unit, and a second block having a monomeric repeating unit. In other embodiments, f is 1, and the polymer is a block copolymer having 1, 2, 3, or 4 monomeric repeating units (having side-chains A², B², C², D²). In some embodiments, each A², B², C² and D² is an amide. In some embodiments, each A², B², C² and D² is an ester.

In some embodiments, the polymer of Formula Y-D is:

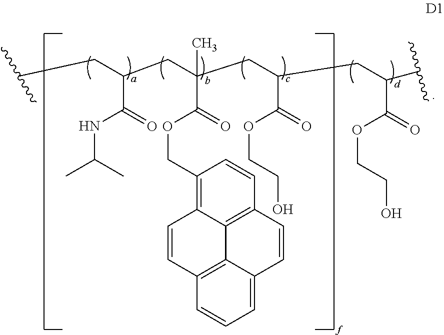

In some embodiments, the ratio of amide side-chains to ester side-chains is about 1:1, 2:1, 3:1, 4:1, 5:1, 6:1, 7:1, 8:1, 9:1, 10:1, 20:1, 30:1, 50:1, 100:1 or greater. In some embodiments, the ratio of ester side-chains to amide side-chains is about 1:1, 2:1, 3:1, 4:1, 5:1, 6:1, 7:1, 8:1, 9:1, 10:1, 20:1, 30:1, 50:1, 100:1 or greater. In some embodiments, the polymer comprises about 0%, about 1%, about 5%, about 10%, about 20%, about 25%, about 30%, about 40%, about 50%, about 60%, about 70%, about 75%, about 80%, about 90%, or about 95% acrylate monomers in a given polymer chain. In some embodiments, the polymer comprises about 0%, about 1%, about 5%, about 10%, about 20%, about 25%, about 30%, about 40%, about 50%, about 60%, about 70%, about 75%, about 80%, about 90%, or about 95% acrylamide monomers in a given polymer chain.

In some embodiments, the polymer comprises 3 monomeric units or more. In some embodiments, the polymer comprises about 5 monomeric units or more, about 10 monomeric units or more, about 15 monomeric units or more, about 20 monomeric units or more, about 30 monomeric units or more, about 50 monomeric units or more, about 100 monomeric units or more, about 200 monomeric units or more, about 300 monomeric units or more, or about 400 monomeric units or more.

In some embodiments, the polymer comprises about 3 monomeric units to about 400 monomeric units. In some embodiments, the polymer comprises about 3 monomeric units to about 4 monomeric units, about 3 monomeric units to about 10 monomeric units, about 3 monomeric units to about 30 monomeric units, about 3 monomeric units to about 50 monomeric units, about 3 monomeric units to about 80 monomeric units, about 3 monomeric units to about 100 monomeric units, about 3 monomeric units to about 120 monomeric units, about 3 monomeric units to about 150 monomeric units, about 3 monomeric units to about 200 monomeric units, about 3 monomeric units to about 400 monomeric units, about 4 monomeric units to about 10 monomeric units, about 4 monomeric units to about 30 monomeric units, about 4 monomeric units to about 50 monomeric units, about 4 monomeric units to about 80 monomeric units, about 4 monomeric units to about 100 monomeric units, about 4 monomeric units to about 120 monomeric units, about 4 monomeric units to about 150 monomeric units, about 4 monomeric units to about 200 monomeric units, about 4 monomeric units to about 400 monomeric units, about 10 monomeric units to about 30 monomeric units, about 10 monomeric units to about 50 monomeric units, about 10 monomeric units to about 80 monomeric units, about 10 monomeric units to about 100 monomeric units, about 10 monomeric units to about 120 monomeric units, about 10 monomeric units to about 150 monomeric units, about 10 monomeric units to about 200 monomeric units, about 10 monomeric units to about 400 monomeric units, about 30 monomeric units to about 50 monomeric units, about 30 monomeric units to about 80 monomeric units, about 30 monomeric units to about 100 monomeric units, about 30 monomeric units to about 120 monomeric units, about 30 monomeric units to about 150 monomeric units, about 30 monomeric units to about 200 monomeric units, about 30 monomeric units to about 400 monomeric units, about 50 monomeric units to about 80 monomeric units, about 50 monomeric units to about 100 monomeric units, about 50 monomeric units to about 120 monomeric units, about 50 monomeric units to about 150 monomeric units, about 50 monomeric units to about 200 monomeric units, about 50 monomeric units to about 400 monomeric units, about 80 monomeric units to about 100 monomeric units, about 80 monomeric units to about 120 monomeric units, about 80 monomeric units to about 150 monomeric units, about 80 monomeric units to about 200 monomeric units, about 80 monomeric units to about 400 monomeric units, about 100 monomeric units to about 120 monomeric units, about 100 monomeric units to about 150 monomeric units, about 100 monomeric units to about 200 monomeric units, about 100 monomeric units to about 400 monomeric units, about 120 monomeric units to about 150 monomeric units, about 120 monomeric units to about 200 monomeric units, about 120 monomeric units to about 400 monomeric units, about 150 monomeric units to about 200 monomeric units, about 150 monomeric units to about 400 monomeric units, or about 200 monomeric units to about 400 monomeric units. In some embodiments, the polymer comprises about 3 monomeric units, about 4 monomeric units, about 10 monomeric units, about 30 monomeric units, about 50 monomeric units, about 80 monomeric units, about 100 monomeric units, about 120 monomeric units, about 150 monomeric units, about 200 monomeric units, or about 400 monomeric units. In some embodiments, the polymer comprises at least about 3 monomeric units, about 4 monomeric units, about 10 monomeric units, about 30 monomeric units, about 50 monomeric units, about 80 monomeric units, about 100 monomeric units, about 120 monomeric units, about 150 monomeric units, or about 200 monomeric units. In some embodiments, polymer comprises at most about 4 monomeric units, about 10 monomeric units, about 30 monomeric units, about 50 monomeric units, about 80 monomeric units, about 100 monomeric units, about 120 monomeric units, about 150 monomeric units, about 200 monomeric units, or about 400 monomeric units. Each monomeric unit comprises a C₁₋₂₀ heteroalkyl side chain.

In some embodiments, the polymer is a polyacrylate, also called, a poly(acrylic ester) or a poly(acrylic acid alkyl ester). In some embodiments, the polymer has a monomeric unit comprising an acrylate, also called, an acrylic ester, or an acrylic acid alkyl ester. In some embodiments, the polymer is a polyacrylamide. In some embodiments, the polymer has a monomeric unit comprising an acrylamide, also called, acrylic amide. In some embodiments, each A², B², C², and D² comprises an acrylate or an acrylamide.

In some embodiments, the monomeric unit has a structure represented by the formula:

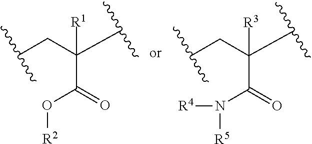

or a pharmaceutically acceptable salt thereof; wherein:
R¹ and R³ are in each instance independently selected from hydrogen and methyl; and
each R² is hydrogen, C₁₋₆ alkyl, C₇₋₂₀ aralkyl, C₁₋₂₀ heteroalkyl, or polyethylene glycol chain containing 1 to 100 ethylene glycol monomers; wherein each said C₁₋₆ alkyl, C₇₋₂₀ aralkyl, and C₁₋₂₀ heteroalkyl is unsubstituted or substituted with one or more groups, wherein each of said one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, or —S(O)₂OH; and
each of R⁴ and R⁵ is independently hydrogen, C₁₋₆ alkyl, C₇₋₂₀ aralkyl, or C₁₋₂₀ heteroalkyl; wherein each said C₁₋₆ alkyl, C₇₋₂₀ aralkyl, and C₁₋₂₀ heteroalkyl is unsubstituted or substituted with one or more groups, wherein each of said one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, or —S(O)₂H.

In some embodiments, R⁴ is hydrogen. In some embodiments, R² and R⁵ are in each instance independently selected from substituted or unsubstituted C₁₋₆ alkyl. In some embodiments, R² and R⁵ are each independently selected from substituted C₁₋₆ alkyl, wherein the alkyl group is substituted with one or more groups selected from —NH₂, —NH₃⁺, —NHC(NH₂⁺)NH₂, —NHCH₃, —N(CH₃)₂, —N(CH₃)₃⁺, —OH, —OCH₃, —S(O)CH₃, —S(O)₂CH₃, and —S(O)₂OH, or a pharmaceutically acceptable salt thereof. In some embodiments, each R² and R⁵ is independently selected from aminoalkyl, hydroxyalkyl, carboxyalkyl, alkoxy, haloalkyl, or any combination thereof. In some embodiments, R² is hydrogen. In some embodiments, R⁴ is hydrogen. In some embodiments, R⁵ is a substituted or unsubstituted C₁₋₆ aminoalkyl group, wherein if the aminoalkyl group is substituted, it is substituted with one or more groups selected from —NH₂, —NH₃⁺, —NHC(NH₂⁺)NH₂, —NHCH₃, —N(CH₃)₂, —N(CH₃)₃⁺, —OH, —OCH₃, —S(O)CH₃, —S(O)₂CH₃, and —S(O)₂OH, or a pharmaceutically acceptable salt thereof.

In some embodiments, the monomeric unit has a structure represented by the formula:

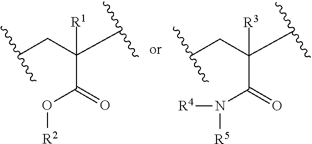

or a pharmaceutically acceptable salt thereof; wherein:
R¹¹ and R¹³ are in each instance independently selected from hydrogen and methyl; and
R¹², R¹⁴, and R¹⁵ are in each instance independently C₁₋₆ alkyl, C₁₋₆ heteroalkyl, C₃₋₁₀ cycloalkyl, or 3- to 10-membered heterocycloalkyl; wherein each alkyl, heteroalkyl, cycloalkyl, and heterocycloalkyl is optionally substituted with one or more groups, each group being independently selected from cycloalkyl, heterocycloalkyl, aryl, heteroaryl, oxo, —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, and —S(O)₂OH, or a pharmaceutically acceptable salt thereof;
or R¹⁴ and R¹⁵ are taken together to form a substituted or unsubstituted heterocycle.

In some embodiments, R¹⁴ is hydrogen. In some embodiments, R¹⁴ and R¹⁵ are taken together to form a substituted or unsubstituted heterocycle (e.g., a 3- to 10-membered heterocyloalkyl ring). In some embodiments, R¹⁴ and R¹⁵ are taken together to form an unsubstituted or substituted aziridine, unsubstituted or substituted azetidine, unsubstituted or substituted pyrrolidine, unsubstituted or substituted piperidine, unsubstituted or substituted piperazine, unsubstituted or substituted morpholine, unsubstituted or substituted azepane, unsubstituted or substituted azocane, or the like. In some embodiments, R¹⁴ is hydrogen, and R¹⁵ is C₁₋₆ alkyl, wherein the C₁₋₆ alkyl is substituted with a heterocycle that is an unsubstituted or substituted aziridine, unsubstituted or substituted azetidine, unsubstituted or substituted pyrrolidine, unsubstituted or substituted piperidine, unsubstituted or substituted piperazine, unsubstituted or substituted morpholine, unsubstituted or substituted azepane, unsubstituted or substituted azocane, or the like.

In some embodiments, the polymer has the structure of Formula Y-B:

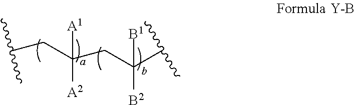

wherein each monomeric unit

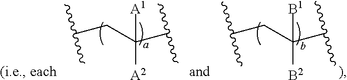

has a structure represented by one of the following formulae:

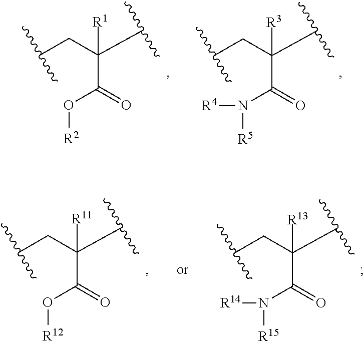

wherein each R¹, R², R³ R⁴, R⁵, R¹¹, R¹², R¹³ R¹⁴, and R¹⁵ is as defined above.

In some embodiments, the polymer has the structure of Formula Y-B1 or Formula Y-B2:

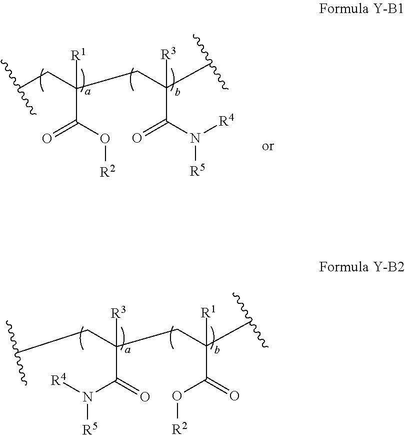

In some embodiments, the monomeric unit is selected from the group consisting of:

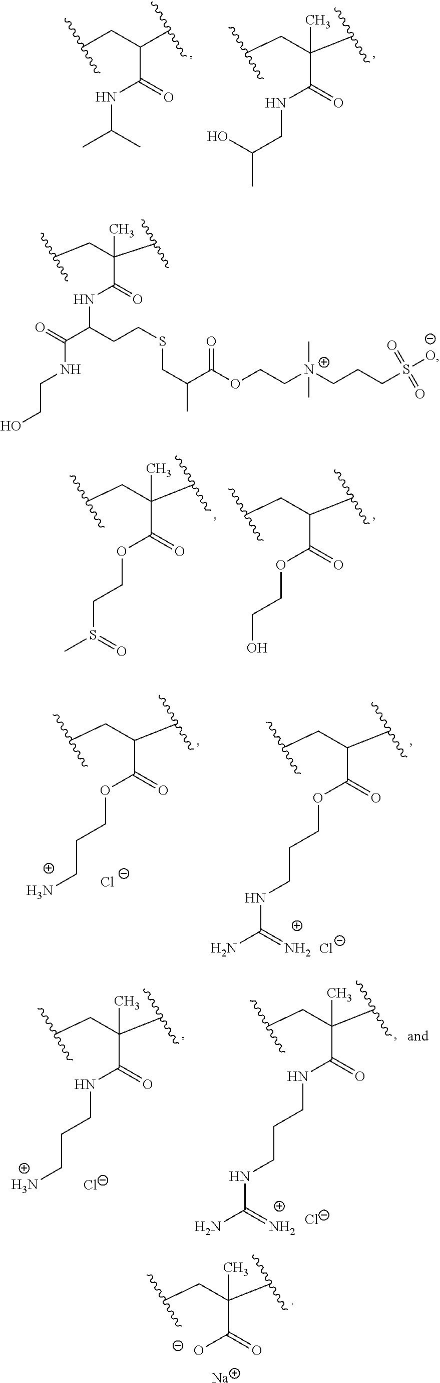

### Functional Groups (“Z”)

Compounds disclosed herein comprise a functional group “Z” which is bonded to the polymer “Y,” conjoined via a bond or optionally separated by a linker. Functional groups can be any functional group known in the art. Examples of functional groups include amines, amides, alcohols, acids, esters, thiols, sulfides, sulfoxides, halogens, nitriles, carbocycles, and heterocycles. A s used herein, functional groups disclosed herein generally comprise a sulfur group (e.g., —SR⁶). In some embodiments, a functional group comprises a reactive group, a charged group, a detectable group, a capping group, or a combination thereof.

A functional group may be or comprise a reactive group, a charged group, a detectable group, a capping group, a binding group, a peptide group, a therapeutic group, a chelating group, a temperature-sensitive group, a light-sensitive group, a radioactive group, a cytotoxic group, or a combination thereof.

In some embodiments, the functional group is a thiol or a sulfhydryl, e.g., —SH. In some embodiments, the functional group is a sulfide, e.g., —SR⁶. In some embodiments, the functional group is a hydroxyl, e.g., —OH. In some embodiments, the functional group is an ether, e.g., —OR⁶. In some embodiments, the functional group is a sulfoxide, e.g., —S(O)R⁶, or a sulfone, e.g., —S(O)₂R⁶.

In some embodiments, R⁶ is a group consisting of 1 to about 200 atoms selected from hydrogen, halogen, C, N, O, and S. In some embodiments, R⁶ is a heteroalkyl group. In some embodiments, R⁶ is a C₁₋₁₀₀ heteroalkyl group. In some embodiments, R⁶ is a C₁₋₂₀ heteroalkyl group. In some embodiments, R⁶ comprises a sulfur atom having connected thereto an alkyl group or heteroalkyl group, each consisting of 1 to about 200 atoms, wherein the non-carbon atoms of the heteroalkyl group are selected from hydrogen, halogen, nitrogen, oxygen, and sulfur. Salts of these groups, e.g., sodium salts, lithium salts, potassium salts, magnesium salts, calcium salts, chloride salts, nitrates salts, phosphate salts, and the like, are considered within the scope of the invention.

In some embodiments, R⁶ comprises a reactive group, a charged group, a detectable group, a peptide group, a capping group, or a combination thereof.

In some embodiments, the reactive group of R⁶ comprises an azide or an alkyne. In some embodiments, the reactive group of R⁶ is capable of reacting with another molecule comprising an alkyne or an azide via “click” chemistry or “click” reaction, in particular, [3+2] cycloadditions, such as Huisgen 1,3-dipolar cycloaddition, copper(I)-catalyzed azide-alkyne cycloaddition (CuAAc), strain-promoted azide-alkyne cycloaddition (SPAAC), strain-promoted alkyne-nitrone cycloaddition (SPANC). In some embodiments, the alkyne is a cyclooctyne, such as dibenzylcyclooctyne, biarylazacyclooctynone, and fluorinated cyclooctyne, or a bicyclononyne.

In some embodiments, the charged group of R⁶ comprises one or more cationic groups. In some embodiments, the one or more cationic groups of R⁶ comprise cyclic amines, primary amines, guanidines, or a combination thereof. In some embodiments, the charged group of R⁶ comprises a plurality of cationic groups (e.g., 3 or more cationic groups). Cationic groups as described herein may comprise amines or other organic groups capable of stably holding a positive or partially positive charge at physiologically relevant pH. For example, primary, secondary, tertiary, or quaternary amines may all be considered cationic groups. In particular, quaternary amines possess a positive (cationic) charge irrespective of protonation status and may be useful in the preparation of compounds described herein.

In some embodiments, the detectable group of R⁶ comprises a fluorophore, a dye, or a Förster resonance energy transfer (FRET) donor or acceptor. In some embodiments, the fluorophore (also called a fluorochrome, a chromophore, or a fluorescent probe), dye, or FRET donor or acceptor, is a fluorescent chemical compound that can re-emit light upon light excitation. Examples of a fluorophore, a dye, a FRET donor or acceptor, but are not limited to, organic dyes (e.g., fluorescein, rhodamine, coumarin, and their derivatives), biological fluorophores (e.g., green fluorescent protein, phycoerythrin, allophycocyanin) and quantum dots.

In some embodiments, the detectable group of R⁶ comprises a fluorescein compound. In some embodiments, the detectable group of R⁶ comprises a rhodamine compound. In some embodiments, the detectable group of R⁶ comprises a coumarin compound. In some embodiments, the detectable group comprises a derivative of a fluorescein, rhodamine, or coumarin compound. In some embodiments, the detectable group comprises or is derived from a fluorescent azide (e.g., azidefluor-488).

In some embodiments, the detectable group comprises a near-infrared fluorescent probe, which is a molecule or a portion of a molecule that emits a signal in response to light in the near-infrared portion of the spectrum. Other detectable groups considered within the scope of the invention include molecules or portions of molecules that emit a signal in response to an excitation source comprising infrared, near-infrared, visible, or ultraviolet light.

In some embodiments, the capping group of R⁶ is an inert group. In some embodiments, the inert group is a chemically-inert group or a chemically-nonreactive group. In some embodiments, the capping group is a hydrocarbon (e.g., C₁₋₂₀ alkyl, such as a methyl, ethyl, or propyl group). In some embodiments, the capping group is a thiol.

The functional group may comprise a binding group. For example, a binding group may be a small molecule capable of binding another atom or molecule. Binding groups could include chelators, which are configured to bind an atom such as a metal. In a specific example, a compound may comprise a functional group that is a chelating agent known in the art (e.g., DOTA, DOTA-TATE, DOTATOC), optionally chelated to a metal atom. The metal atom may be an ion or an oxide. Specifically, the metal atom may be a lanthanide or an actinide. In some embodiments, the metal atom is an alpha emitter, a beta emitter, or a gamma emitter. In some embodiments, the metal atom is an actinium, yttrium, gadolinium, actinium, lutetium, or an isotope thereof.

The functional group may also comprise a peptide group. In some instances, the functional group comprises a targeting group (e.g., wherein the peptide group is a targeting group). The targeting group may be a peptide. A targeting group may be a cyclic peptide. In a specific example, the functional group comprises a cyclic peptide that targets integrin receptors (e.g., αᵥβ₃-integrin receptors). For example, the functional group may comprise an RGD peptide. Radiolabeled cyclic peptides containing the (Arg-Gly-Asp) RGD sequence have been reported for use in positron emission tomography (PET) imaging, single-photon emission computed tomography (SPECT) imaging, and targeted radionuclide therapy of cancer. Any RGD peptide known in the art could be appended as a functional group as disclosed herein. In a specific example, a functional group comprising a cyclo(-Arg-Gly-Asp-D-Phe-Lys) (“cRGDtK”) group was synthesized. Also considered within the scope of the invention are radiolabeled analogs, salts, or radioisotopic derivatives thereof. In examples having a functional group that comprises a cRGDfK group, the cRGDfK group may be linked to the polymer (Y), for example, via a lysine nitrogen atom, and optionally conjoined via a linker. As used herein, the term “linker” includes any alkyl, heteroalkyl, cyclyl, or heterocyclyl group, or any combination thereof, comprising 1 to 100 atoms selected from C, H, N, O, and S. Linkers may comprise, for example, maleimides, ethylene units, propylene units, amides, amines, esters, ethers, thioethers, polyethylene glycol chains, alkyl chains, click reagents, peptides, or any combination thereof. In some embodiments, the functional group (optionally comprising a linker), is any one of the groups (Z) shown in table 1.

The functional group may comprise a therapeutic group. For example, the functional group could comprise a drug linked via a cleavable linker. The therapeutic group could also be a binding agent, or a radiotherapeutic group comprising a chelator and radioisotope as discussed above. In some embodiments, the functional group comprises a chelating group. Chelating groups may be coordinated with a metal ion. Chelating groups may be useful either as therapeutics, or as detectable agents (e.g., in PET or SPECT imaging). A chelating group may be cyclic or non-cyclic, and often comprises two or more basic amines or acidic carboxylates. One example of a chelating functional group is the aminopolycarboxylic acid group, nitrilotriacetic acid (NTA). Additional aminopolycarboxylic acid chelators include NTA, EDTA, DTPA, EGTA, BAPTA, NOTA, DOTA, and derivatives thereof.

In some embodiments, the functional group (Z) is selected from:

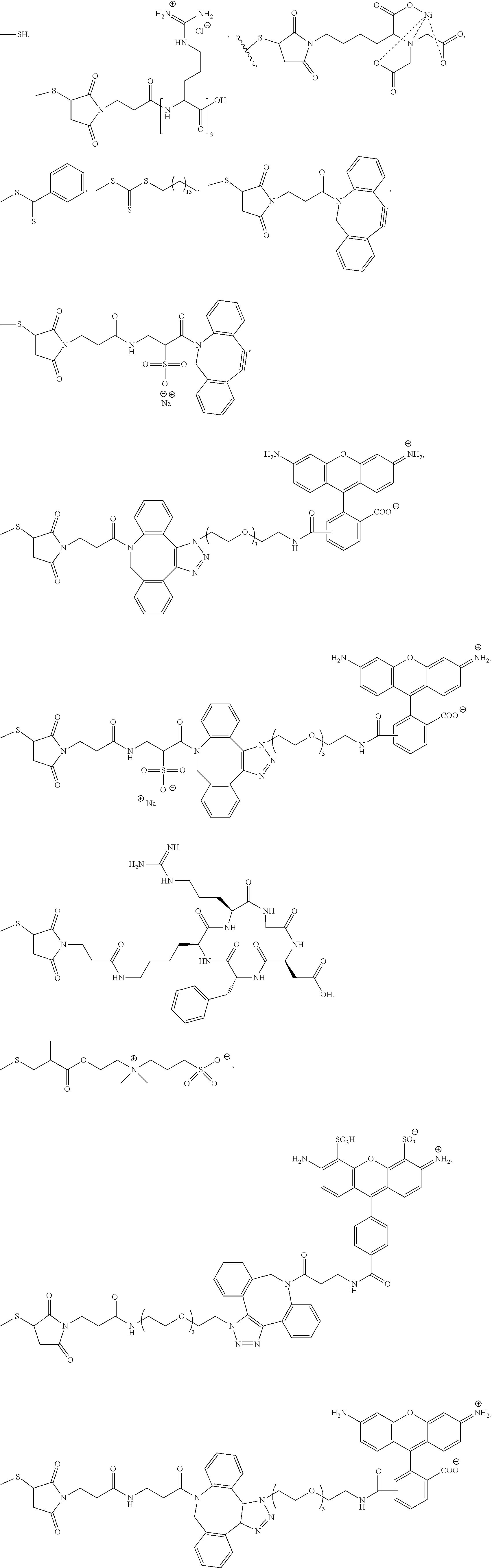

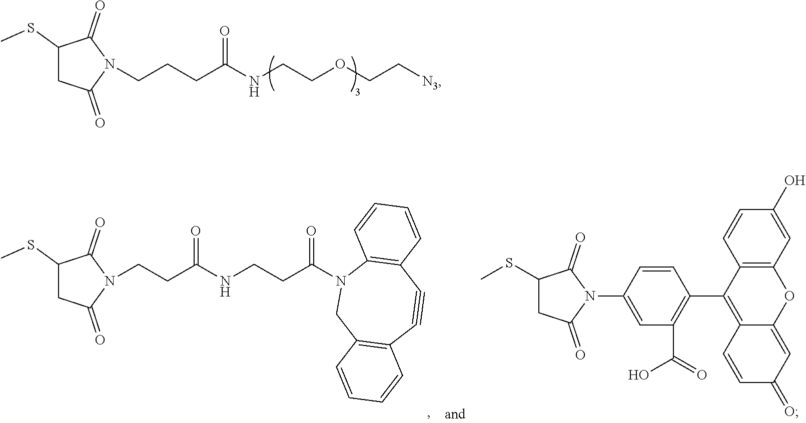

wherein said functional group is bonded to said polymer via the sulfur atom at the left-hand side of each of the preceding structures.

In another aspect, provided herein are compounds of Formula I:

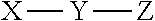

or a pharmaceutically acceptable salt thereof; wherein:
X is a lipid selected from X-A, X-B, and X-C:

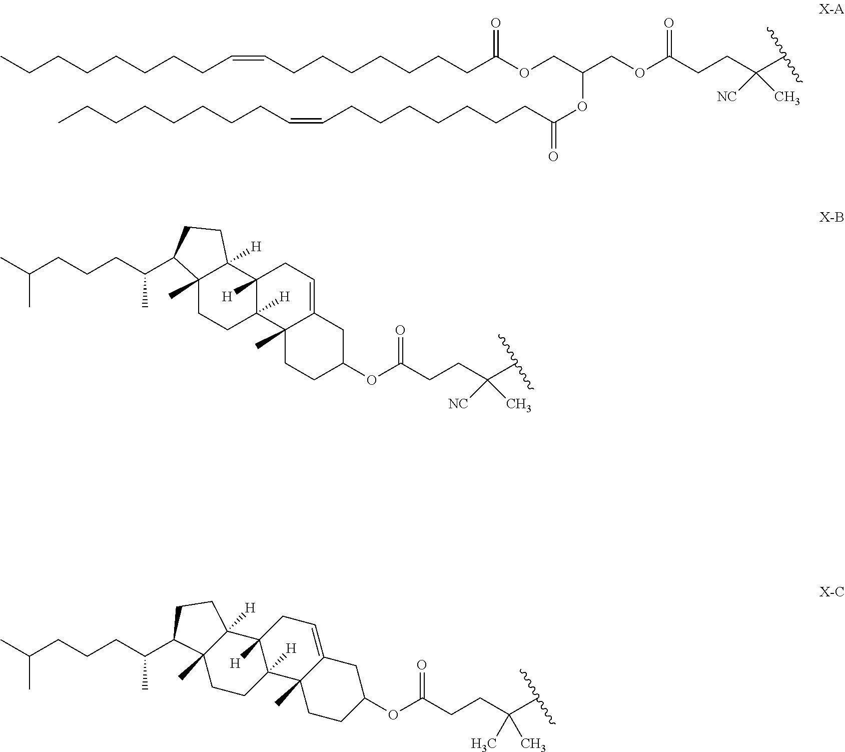

Y is a polymer having a structure as described under Formula Y-A, Y-B, Y-C, or Y-D; and
Z is a functional group as illustrated below.

Specific embodiments of the present invention are disclosed in Table 1 below.

TABLE 1

| Compound | X | Y | (Z) |
|---|---|---|---|
| 1 | X-A | A1 a = 94 | 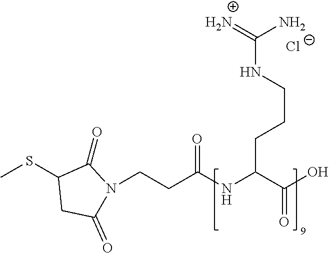 |

| | | | |
|---|---|---|---|
| 2 | X-A | A1 a = 94 | 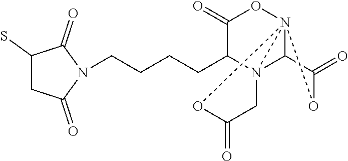 |

| | | | |
|---|---|---|---|
| 3 | X-A | A1 a = 94 | 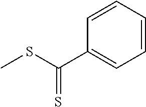 |

| | | | |
|---|---|---|---|
| 4 | X-A | B1 | —SH |
| | | a = 120 | |
| | | b = 3-10 | |
| 5 | X-B | B1 | —SH |
| | | a = 50-170 | |
| | | b = 1-200 | |

| | | | |
|---|---|---|---|
| 6 | X-C | C1 a = 82-150 b = 1-9 c = 0-260 | 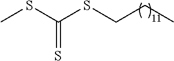 |

| | | | |
|---|---|---|---|
| 7 | X-C | D1 | —SH |
| | | a = 86-108- | |
| | | b = 1-2 − | |
| | | c = --1-7 | |
| | | e = 30~150-- | |
| 8 | X-A | A2 a = 72 | 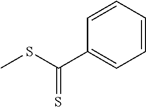 |
| 9 | X-A | A2 a = 72 | 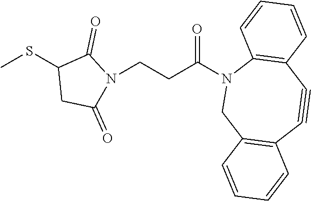 |
| 10 | X-A | A2 a = 72 | 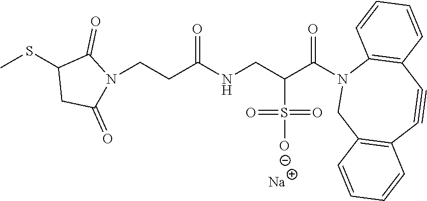 |
| 11 | X-A | A2 a = 72 | 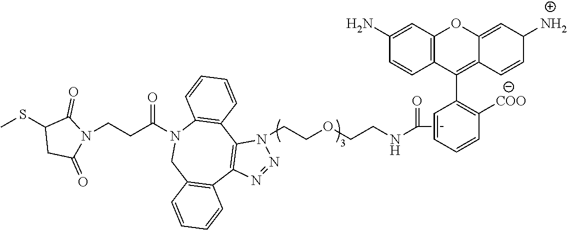 |
| 12 | X-A | A2 a = 72 | 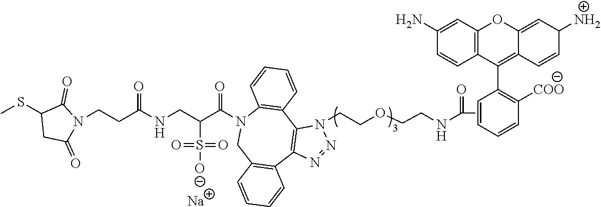 |
| 13 | X-A | A2 a = 72 | 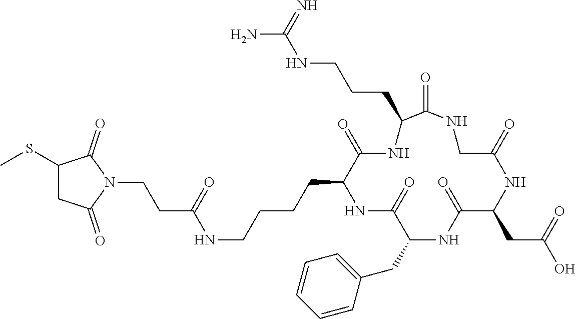 |
| 14 | X-A | A3 a = 47 | 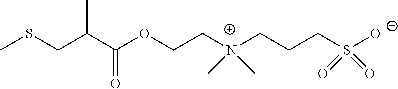 |
| 15 | X-A | A4 a = 114 | 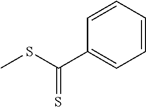 |
| 16 | X-C | A5 | -SH |
| | | a = 45-300 | |
| 17 | X-C | B2 | -SH |
| | | a = 45-100 | |
| | | b = 1-10 | |
| 18 | X-A | B2 | -SH |
| | | a = -32-200 | |
| | | b = 3-11 | |
| 19 | X-A | A6 a = 100 | 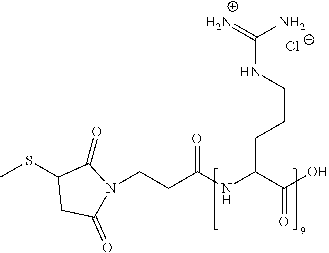 |
| 20 | X-A | A6 a = 100 | 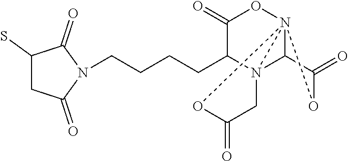 |
| 21 | X-A | A6 | —SH |
| | | a = 100 | |
| 22 | X-A | A7 | —SH |
| | | a = 100 | |
| 23 | X-A | A8 | —SH |
| | | a = 17 | |
| 24 | X-A | A9 | —SH |
| | | a = 43 | |
| 25 | X-A | A9 | —SH |
| | | a = 43 | |
| 26 | X-B | A1 a = 94 | 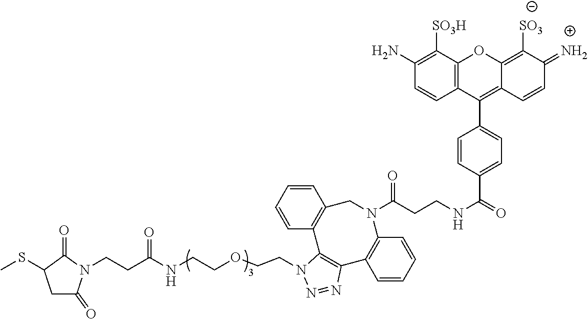 |
| 27 | X-B | A1 a = 94 | 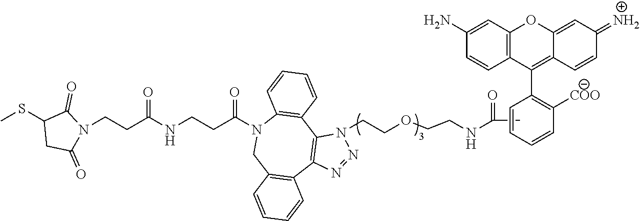 |
| 28 | X-B | A1 a = 94 | 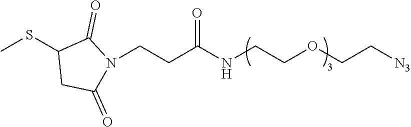 |
| 29 | X-B | A1 a = 94 | 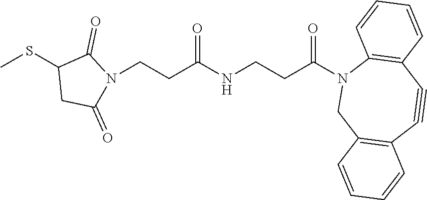 |
| 30 | X-A | A6 a = 100 | 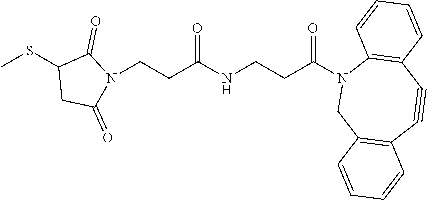 |
| 31 | X-A | A10 a = 97 | 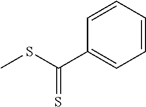 |
| 32 | X-A | A10 a = 97 | 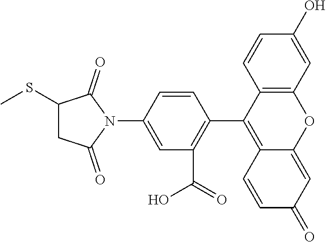 |

In some embodiments, the compound is configured to encapsulate or complex with nucleic acids in aqueous solution. In some embodiments, the compound is substantially non-toxic.

In some embodiments, the compound is biodegradable. In some embodiments, the compound comprises a molecular weight of about 1 kDa to about 100 kDa. In some embodiments, the compound comprises a molecular weight of about 1 kDa to about 10 kDa, about 1 kDa to about 20 kDa, about 1 kDa to about 50 kDa, about 1 kDa to about 70 kDa, about 1 kDa to about 90 kDa, about 1 kDa to about 100 kDa, about 10 kDa to about 20 kDa, about 10 kDa to about 50 kDa, about 10 kDa to about 70 kDa, about 10 kDa to about 90 kDa, about 10 kDa to about 100 kDa, about 20 kDa to about 50 kDa, about 20 kDa to about 70 kDa, about 20 kDa to about 90 kDa, about 20 kDa to about 100 kDa, about 50 kDa to about 70 kDa, about 50 kDa to about 90 kDa, about 50 kDa to about 100 kDa, about 70 kDa to about 90 kDa, about 70 kDa to about 100 kDa, or about 90 kDa to about 100 kDa. In some embodiments, the compound comprises a molecular weight of about 1 kDa, about 10 kDa, about 20 kDa, about 50 kDa, about 70 kDa, about 90 kDa, or about 100 kDa. In some embodiments, the compound comprises a molecular weight of at least about 1 kDa, about 10 kDa, about 20 kDa, about 50 kDa, about 70 kDa, or about 90 kDa. In some embodiments, the compound comprises a molecular weight of at most about 10 kDa, about 20 kDa, about 50 kDa, about 70 kDa, about 90 kDa, or about 100 kDa.

### Liposomes & Nanoparticles

Lipid-polymer compounds disclosed herein may be useful in the preparation of liposomes and/or lipid nanoparticles. For example, PLips disclosed herein can serve various purposes. For example, PLips disclosed herein may add stability or other utility to lipid nanoparticles (LNPs). Lipid nanoparticles are generally spherical vesicles made from ionizable lipids, which can be positively charged at low pH (enabling RNA complexation) and neutral at physiological pH (reducing potential toxic effects, as compared with positively charged lipids, such as liposomes). Owing to their size and properties, lipid nanoparticles can be taken up by cells via endocytosis, and the ionizability of the lipids at low pH may facilitate endosomal escape, which allows release of a payload into the cytoplasm of a target cell. The PLips disclosed herein may be used in the preparation of a lipid nanoparticle (LNP). Accordingly, the use of the PLips disclosed herein in the preparation of a lipid nanoparticle, as well as any lipid nanoparticle comprising a PLip (or “compound) disclosed herein, is within the scope of the invention. PLips disclosed herein may aid in the stability of a LNP (e.g., a stabilizing PLip). In many cases, a PLip may have more than one utility. For example, a PLip may be both a stabilizing and reactive PLip, meaning that the PLip has a stabilizing polymeric block, and the functional group comprises a reactive moiety (e.g., a strained cyclooctyne or an azide). Similarly, a PLip may be cationic and comprise a reactive moiety. In some instances, the reactive moiety is reacted with a dye to form a fluorescently labeled LNP. Stabilizing PLips may be used as a replacement for PEG, which has demonstrated toxicity and problematic side-effects in some biological systems. A stabilizing PLip may comprise PEG side-chains, or a heteroalkyl acrylate or acrylamide side-chain.

Cationic PLips may comprise a plurality of cationic groups. In some instances, a cationic PLip comprises between about 3 and about 20 cationic monomeric units. Cationic PLips can, in some embodiments, replace cationic lipids used in LNP formulations. Cationic PLips may be useful as transfection reagents (e.g., whereby the cationic amine (N) interacts non-covalently with a phosphate backbone (P) of a nucleic acid). In some instances, the N:P ratio is about 1:1, about 2:1, about 3:1, about 4:1, about 5:1, about 8:1, about 10:1, about 16:1, about 20:1, about 32:1, about 50:1, about 64:1, about 100:1, about 128:1, about 150:1, about 200:1, or more. In specific examples, the N:P ratio is between about 1:4 and about 128:1. In more specific examples, the N:P ratio is about 4:1 to about 16:1. A cationic PLip may have a positive charge. In some examples, a cationic PLip has a positive charge of about 1, 2, 3, 4, 5, 6, 7, 8, 9, 10, 11, 12, 13, 14, 15, 16, 17, 18, 19, 20, 21, 22, 23, 24, or more. The positive charge may directly correlate with the polymer length of a polymer comprising cationic side-chains. For example, a cationic PLip with a polymer comprising 19 cationic monomeric units may have a +19 charge. Similarly, a cationic PLip with a polymer comprising 13 cationic monomeric units may have a +13 charge. In other instances, a polymer may comprise two blocks of repeating monomeric units, wherein a first block comprises stabilizing side-chains, and wherein the second block comprises cationic side-chains. In such a case, the stabilizing polymer block may exceed the length of the cationic block 2-fold, 3-fold, 4-fold, 5-fold, 8-fold, 10-fold, 16-fold, 20-fold, 32-fold, 50-fold, 64-fold, or 100-fold or more. For example, in some instances a polymer comprises a stabilizing block of monomeric units comprising about 100 to about 200 (e.g., about 170 to about 190), and a second cationic block of monomeric units comprising about 3 to about 30 cationic monomeric units (e.g., about 3 to about 10 monomeric units). In alternate embodiments, a polymer comprises a block of alternating monomeric units, wherein the first monomeric unit has a stabilizing (non-charged) side-chain, and wherein the second monomeric unit has a cationic side-chain.

Lipid-polymer compounds (e.g., cationic PLips) disclosed herein may have utility as in the preparation of nanoparticles and/or transfection reagents. As used herein, a “nanoparticle” generally refers to an aggregate of compounds that can form a vesicle. As described herein, a nanoparticle may be configured to encapsulate one or more nucleic acid molecule, thereby generating a transfection reagent, i.e., a reagent configured to transmit genetic information into a host cell. A nanoparticle may be, for example, configured to encapsulate or form a complex with nucleic acids (e.g., DNA, RNA, etc.). A nanoparticle prepared as described herein may be configured for said encapsulation or said complexation of said nucleic acids at a 0.3:1 to 100:1 (weight:weight) ratio. For example, a transfection reagent encapsulate or complex with one or more nucleic acids at a 0.5:1 to 100:1, 1:1 to 100:1, 5:1 to 100:1, 10:1 to 100:1, 20:1 to 100:1, 30:1 to 100:1, 40:1 to 100:1, 50:1 to 100:1, 60:1 to 100:1, 70:1 to 100:1, 80:1 to 100:1, 90:1 to 100:1, or 95:1 to 100:1 (weight:weight) ratio. Generally, a transfection reagent refers to combination of a nanoparticle and genetic materials, and a nanoparticle refers to the vesicle itself. Furthermore, a nanoparticle may comprise a plurality of compounds or PLips disclosed herein. Advantages of nanoparticles and transfection reagents comprising compounds or PLips disclosed herein are made evident throughout the examples.

In some embodiments, the encapsulation or the complexation of the nucleic acids increases a half-life of said nucleic acid by at least 2-fold in aqueous or physiological conditions. In some embodiments, the encapsulation or the complexation of the nucleic acids increases a half-life of said nucleic acid by at least 1.1-fold, at least 1.3-fold, at least 1.5-fold, at least 1.7-fold, at least 2 fold, at least 2.5 fold, or at least 3 fold in aqueous or physiological conditions.

In some embodiments, the encapsulation or complexation inhibits nuclease digestion of the nucleic acid. In some embodiments, the encapsulation or complexation of the nucleic acids generates transfection complexes with average sizes of 20-2000 nm. In some embodiments, the encapsulation or complexation of the nucleic acids generates transfection complexes with average sizes of about 20 nm to about 2,000 nm. In some embodiments, the encapsulation or complexation of the nucleic acids generates transfection complexes with average sizes of about 20 nm to about 30 nm, about 20 nm to about 50 nm, about 20 nm to about 100 nm, about 20 nm to about 200 nm, about 20 nm to about 300 nm, about 20 nm to about 400 nm, about 20 nm to about 500 nm, about 20 nm to about 1,000 nm, about 20 nm to about 1,500 nm, about 20 nm to about 2,000 nm, about 30 nm to about 50 nm, about 30 nm to about 100 nm, about 30 nm to about 200 nm, about 30 nm to about 300 nm, about 30 nm to about 400 nm, about 30 nm to about 500 nm, about 30 nm to about 1,000 nm, about 30 nm to about 1,500 nm, about 30 nm to about 2,000 nm, about 50 nm to about 100 nm, about 50 nm to about 200 nm, about 50 nm to about 300 nm, about 50 nm to about 400 nm, about 50 nm to about 500 nm, about 50 nm to about 1,000 nm, about 50 nm to about 1,500 nm, about 50 nm to about 2,000 nm, about 100 nm to about 200 nm, about 100 nm to about 300 nm, about 100 nm to about 400 nm, about 100 nm to about 500 nm, about 100 nm to about 1,000 nm, about 100 nm to about 1,500 nm, about 100 nm to about 2,000 nm, about 200 nm to about 300 nm, about 200 nm to about 400 nm, about 200 nm to about 500 nm, about 200 nm to about 1,000 nm, about 200 nm to about 1,500 nm, about 200 nm to about 2,000 nm, about 300 nm to about 400 nm, about 300 nm to about 500 nm, about 300 nm to about 1,000 nm, about 300 nm to about 1,500 nm, about 300 nm to about 2,000 nm, about 400 nm to about 500 nm, about 400 nm to about 1.000 nm, about 400 nm to about 1,500 nm, about 400 nm to about 2,000 nm, about 500 nm to about 1,000 nm, about 500 nm to about 1,500 nm, about 500 nm to about 2,000 nm, about 1,000 nm to about 1,500 nm, about 1,000 nm to about 2,000 nm, or about 1,500 nm to about 2,000 nm. In some embodiments, the encapsulation or complexation of the nucleic acids generates transfection complexes with average sizes of about 20 nm, about 30 nm, about 50 nm, about 100 nm, about 200 nm, about 300 nm, about 400 nm, about 500 nm, about 1,000 nm, about 1,500 nm, or about 2,000 nm. In some embodiments, the encapsulation or complexation of the nucleic acids generates transfection complexes with average sizes of at least about 20 nm, about 30 nm, about 50 nm, about 100 nm, about 200 nm, about 300 nm, about 400 nm, about 500 nm, about 1,000 nm, or about 1,500 nm. In some embodiments, the encapsulation or complexation of the nucleic acids generates transfection complexes with average sizes of at most about 30 nm, about 50 nm, about 100 nm, about 200 nm, about 300 nm, about 400 nm, about 500 nm, about 1,000 nm, about 1,500 nm, or about 2,000 nm. In some embodiments, the encapsulation or complexation of the nucleic acids generates transfection complexes with average radii of about 100 nm to about 300 nm (e.g., about 100 to about 200 nm). In some embodiments, a LNP has an average radius of about 100 nm, about 110 nm, about 120 nm, about 130 nm, about 140 nm, about 150 nm, about 160 nm, about 170 nm, about 180 nm, about 190 nm, or about 200 nm. In some embodiments, a temperature-sensitive LNP can induce a temperature-dependent swelling of the LNP, for example, from about 180 nm radius to about 300 nm radius. The temperature-sensitive LNP swelling can, in some examples, be a reversible process (e.g., whereby less than about 10%, less than about 5%, less than about 3%, less than about 2% or less than about 1% of the LNP particles degrade or aggregate after heating (e.g., to about 45° C.). The process may be entirely or almost entirely reversible (i.e., less than about 2% loss of LNP per thermal cycle).

In some embodiments, the encapsulation or the complexation comprises adsorption of at least a subset of said nucleic acids to a surface of said transfection reagent. In some embodiments, the encapsulation or the complexation of the nucleic acids generates transfection complexes configured for cellular uptake. In some embodiments, the cellular uptake comprises endocytosis.

In some embodiments, the encapsulation or the complexation of the nucleic acids generates transfection complexes comprising aqueous solubilities of at least 5 μg/mL. In some embodiments, the encapsulation or the complexation of the nucleic acids generates transfection complexes comprising aqueous solubilities of at least 1 μg/mL, at least 5 μg/mL, at least 10 μg/mL, at least 50 μg/mL, at least 100 μg/mL, at least 200 μg/mL, at least 500 μg/mL, at least 1000 μg/mL, at least 1500 μg/mL, at least 2000 μg/mL, at least 2500 μg/mL, at least 3000 μg/mL, at least 3500 μg/mL, at least 4000 μg/mL, at least 4500 μg/mL, or at least 5000 μg/mL.

In some embodiments, the encapsulation or the complexation of the nucleic acids generates transfection complexes comprising aqueous solubilities of about 10 μg/mL to about 50 μg/mL. In some embodiments, the encapsulation or the complexation of the nucleic acids generates transfection complexes comprising aqueous solubilities of about 1 μg/mL to about 100 μg/mL. In some embodiments, the encapsulation or the complexation of the nucleic acids generates transfection complexes comprising aqueous solubilities of about 1 μg/mL to about 5 μg/mL, about 1 μg/mL to about 10 μg/mL, about 1 μg/mL to about 20 μg/mL, about 1 μg/mL to about 30 μg/mL, about 1 μg/mL to about 40 μg/mL, about 1 μg/mL to about 50 μg/mL, about 1 μg/mL to about 100 μg/mL, about 5 μg/mL to about 10 μg/mL, about 5 μg/mL to about 20 μg/mL, about 5 μg/mL to about 30 μg/mL, about 5 μg/mL to about 40 μg/mL, about 5 μg/mL to about 50 μg/mL, about 5 μg/mL to about 100 μg/mL, about 10 μg/mL to about 20 μg/mL, about 10 μg/mL to about 30 μg/mL, about 10 μg/mL to about 40 μg/mL, about 10 μg/mL to about 50 μg/mL, about 10 μg/mL to about 100 μg/mL, about 20 μg/mL, to about 30 μg/mL, about 20 μg/mL to about 40 μg/mL, about 20 μg/mL to about 50 μg/mL, about 20 μg/mL to about 100 μg/mL, about 30 μg/mL to about 40 μg/mL, about 30 μg/mL to about 50 μg/mL, about 30 μg/mL to about 100 μg/mL, about 40 μg/mL to about 50 μg/mL, about 40 μg/mL to about 100 μg/mL, or about 50 μg/mL to about 100 μg/mL. In some embodiments, the encapsulation or the complexation of the nucleic acids generates transfection complexes comprising aqueous solubilities of about 1 μg/mL, about 5 μg/mL, about 10 μg/mL, about 20 μg/mL, about 30 μg/mL, about 40 μg/mL, about 50 μg/mL, or about 100 μg/mL. In some embodiments, the encapsulation or the complexation of the nucleic acids generates transfection complexes comprising aqueous solubilities of at least about 1 μg/mL, about 5 μg/mL, about 10 μg/mL, about 20 μg/mL, about 30 μg/mL, about 40 μg/mL, or about 50 μg/mL. In some embodiments, the encapsulation or the complexation of the nucleic acids generates transfection complexes comprising aqueous solubilities of at most about 5 μg/mL, about 10 μg/mL, about 20 μg/mL, about 30 μg/mL, about 40 μg/mL, about 50 μg/mL, or about 100 μg/mL.

In some embodiments, the encapsulation or the complexation of the nucleic acids generates transfection complexes comprising aqueous solubilities of about 5 μg/mL to about 5,000 μg/mL. In some embodiments, the encapsulation or the complexation of the nucleic acids generates transfection complexes comprising aqueous solubilities of about 5 μg/mL to about 50 μg/mL, about 5 μg/mL to about 100 μg/mL, about 5 μg/mL to about 500 μg/mL, about 5 μg/mL to about 1,000 μg/mL, about 5 μg/mL to about 2,000 μg/mL, about 5 μg/mL to about 3,000 μg/mL, about 5 μg/mL to about 4,000 μg/mL, about 5 μg/mL to about 5,000 μg/mL, about 50 μg/mL to about 100 μg/mL, about 50 μg/mL to about 500 μg/mL, about 50 μg/mL to about 1,000 sig/mL, about 50 μg/mL to about 2,000 μg/mL, about 50 μg/mL to about 3,000 μg/mL, about 50 μg/mL to about 4,000 μg/mL, about 50 μg/mL to about 5,000 μg/mL, about 100 μg/mL to about 500 μg/mL, about 100 μg/mL to about 1,000 μg/mL, about 100 μg/mL to about 2,000 μg/mL, about 100 μg/mL to about 3,000 μg/mL, about 100 μg/mL to about 4,000 μg/mL, about 100 μg/mL to about 5,000 μg/mL, about 500 μg/mL to about 1,000 μg/mL, about 500 μg/mL to about 2,000 μg/mL, about 500 μg/mL to about 3,000 μg/mL, about 500 μg/mL to about 4,000 μg/mL, about 500 μg/mL to about 5,000 μg/mL, about 1,000 μg/mL to about 2,000 μg/mL, about 1,000 μg/mL to about 3,000 μg/mL, about 1,000 μg/mL to about 4,000 μg/mL, about 1,000 μg/mL to about 5,000 μg/mL, about 2,000 μg/mL to about 3,000 μg/mL, about 2,000 μg/mL to about 4,000 μg/mL, about 2,000 μg/mL to about 5,000 μg/mL, about 3,000 μg/mL to about 4,000 μg/mL, about 3,000 μg/mL to about 5,000 μg/mL, or about 4,000 μg/mL to about 5,000 μg/mL. In some embodiments, the encapsulation or the complexation of the nucleic acids generates transfection complexes comprising aqueous solubilities of about 5 μg/mL, about 50 μg/mL, about 100 μg/mL, about 500 μg/mL, about 1,000 μg/mL, about 2,000 μg/mL, about 3,000 μg/mL, about 4,000 μg/mL, or about 5,000 μg/mL. In some embodiments, the encapsulation or the complexation of the nucleic acids generates transfection complexes comprising aqueous solubilities of at least about 5 μg/mL, about 50 μg/mL, about 100 μg/mL, about 500 μg/mL, about 1,000 μg/mL, about 2,000 μg/mL, about 3,000 μg/mL, or about 4,000 μg/mL. In some embodiments, the encapsulation or the complexation of the nucleic acids generates transfection complexes comprising aqueous solubilities of at most about 50 μg/mL, about 100 μg/mL, about 5(0) μg/mL, about 1,000 μg/mL, about 2,000 μg/mL, about 3,000 μg/mL, about 4,000 μg/mL, or about 5,000 μg/mL.

In some embodiments, the compound has a dispersity (i.e., molecular weight distribution) of about 2.0 or less. In some embodiments, the compound has a dispersity of about 1.5 or less. In some embodiments, the compound has a dispersity of about 1.3 or less. In some embodiments, the compound has a dispersity of about 1.2 or less. In some embodiments, the compound has a dispersity of at least 1.0. In some embodiments, the compound has a dispersity of at least 1.1. In some embodiments, the compound has a dispersity of at least 1.2. In some embodiments, the compound has a dispersity of at least 1.3. In some embodiments, the compound has a dispersity of at least 1.4. In some embodiments, the compound has a dispersity of at least 1.5. In some embodiments, the compound has a dispersity of about 1.1 to about 1.5. In some embodiments, the compound has a dispersity of about 1.2 to about 1.5. In some embodiments, the compound has a dispersity of about 1.3 to about 1.5. In some embodiments, the compound has a dispersity of about 1.2 to about 1.8. In some embodiments, the compound has a dispersity of about 1.5 to about 1.8. In some embodiments, the compound has a dispersity of about 1.5 to about 2.0. In some embodiments, the compound has a dispersity of about 1.8 to about 2.0. In some embodiments, the compound has a dispersity of about 1.8 to about 2.3. In some embodiments, the compound has a dispersity of about 2.0 to about 2.3. In some embodiments, the compound has a dispersity of about 2.0 to about 2.5. In some embodiments, the compound has a dispersity of about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 3.0 or less.

### Transfection Reagents

In an aspect, provided herein are reagents (e.g., transfection reagents) comprising a compound disclosed herein and a nucleic acid. Transfection reagents can act as delivery vehicles for a payload (e.g., a biologically active compound (e.g., a nucleic acid)). The payload may be specifically matched with a given transfection reagents, or a transfection reagent may be capable of delivering a variety of payloads. Transfection reagents may offer multiple advantageous properties to assist in the efficient delivery of a payload to a target (e.g., a target cell). For example, a transfection reagent can comprise a liposome or a lipid nanoparticle (LNP). Liposomes and LNPs can act as micro-vesicles, having approximately spheroid shape and containing an interior and an exterior. Advantageously, by encapsulating a payload within the interior of the liposome or LNP, the payload can thereby be shielded from biological and chemical processes that may otherwise degrade the payload. Alternatively, an encapsulated payload may reside within a lipid bilayer, rather than within the internal compartment. In still another example, the internal compartment may be tailored to stabilize a particular payload.

In another example, a transfection reagent may form a lipid bilayer around a payload which, upon contacting a target cell, may fuse with the cell and release the payload intracellularly. A transfection reagent may also physically exclude enzymes or ribozymes, or may insulate the internal payload from external changes in environment (e.g., pH, ionic concentration, etc.).

In another aspect, a transfection reagent comprising a compound disclosed herein may interact directly with the payload, e.g., to stabilize or chelate certain groups. For example, a transfection reagent comprising a compound disclosed herein, can form polar, ionic, or other non-covalent interactions with a nucleic acid payload. While some vesicles may contain a simple lipid shell and aqueous internal compartment, other vesicles (e.g., LNPs) can have compounds distributed within the internal space (e.g., interacting with an internalized payload). In a specific example, a transfection reagent comprising compounds disclosed herein both (a) creates a vesicle that separates an internal compartment from the external environment, and (b) encapsulates the internal payload with a compound disclosed herein. Of particular utility, a polymer disclosed herein can comprise a hydrocarbon backbone capable of forming a bilayer, whereas a polymer side-chain can form a stabilizing interaction with a nucleic acid payload (e.g., a phosphate backbone of a nucleic acid payload). A nucleic acid payload may comprise DNA or RNA (e.g., siRNA, saRNA, mRNA, microRNA, etc.).

In still another aspect, a transfection reagent disclosed herein may comprise a lipid-polymer compound having a functional group that acts as a reporter moiety or a reactive moiety. In addition to the abovementioned aspects of stabilizing, shielding, and delivering a payload to the intracellular space of a target cell, transfection reagents comprising reporter compounds or reactive compounds may fuse with the target cell, thereby becoming incorporated or inserted into the target cell. In a particular example, a transfection reagent comprising a polymer-lipid compound with a fluorescent functional group may deliver a nucleic acid payload to a target cell, and upon fusion with the target cell, thereby labeling the target cell with a fluorescent functional group. Similarly, a transfection reagent comprising a reactive functional group (e.g., a click handle) may contact and insert into a target cell, thereby providing a reactive handle on said target cell. Additional functional groups disclosed herein can similarly be incorporated into a target cell, thereby functionalizing said target cell with said functional groups.

A nucleic acid can comprise deoxyribonucleic acid (DNA), ribonucleic acid (RNA), locked nucleic (LNA), peptide nucleic acid (PNA), or a combination thereof. In some embodiments, the nucleic acid comprises about 1 kb to about 100 kb (e.g., about 1 kb to about 2 kb, about 1 kb to about 5 kb, about 1 kb to about 8 kb, about 1 kb to about 10 kb, about 1 kb to about 12 kb, about 1 kb to about 15 kb, about 1 kb to about 20 kb, about 1 kb to about 50 kb, about 1 kb to about 100 kb, about 2 kb to about 5 kb, about 2 kb to about 8 kb, about 2 kb to about 10 kb, about 2 kb to about 12 kb, about 2 kb to about 15 kb, about 2 kb to about 20 kb, about 2 kb to about 50 kb, about 2 kb to about 100 kb, about 5 kb to about 8 kb, about 5 kb to about 10 kb, about 5 kb to about 12 kb, about 5 kb to about 15 kb, about 5 kb to about 20 kb, about 5 kb to about 50 kb, about 5 kb to about 100 kb, about 8 kb to about 10 kb, about 8 kb to about 12 kb, about 8 kb to about 15 kb, about 8 kb to about 20 kb, about 8 kb to about 50 kb, about 8 kb to about 100 kb, about 10 kb to about 12 kb, about 10 kb to about 15 kb, about 10 kb to about 20 kb, about 10 kb to about 50 kb, about 10 kb to about 100 kb, about 12 kb to about 15 kb, about 12 kb to about 20 kb, about 12 kb to about 50 kb, about 12 kb to about 100 kb, about 15 kb to about 20 kb, about 15 kb to about 50 kb, about 15 kb to about 100 kb, about 20 kb to about 50 kb, about 20 kb to about 100 kb, or about 50 kb to about 100 kb). A nucleic acid may comprise about 1 kb, about 2 kb, about 5 kb, about 8 kb, about 10 kb, about 12 kb, about 15 kb, about 20 kb, about 50 kb, or about 100 kb. In some embodiments, the nucleic acid comprises at least about 1 kb, about 2 kb, about 5 kb, about 8 kb, about 10 kb, about 12 kb, about 15 kb, about 20 kb, or about 50 kb. In some embodiments, the nucleic acid comprises at most about 2 kb, about 5 kb, about 8 kb, about 10 kb, about 12 kb, about 15 kb, about 20 kb, about 50 kb, or about 100 kb. In some instances, the nucleic acid comprises about 2 kb to about 20 kb. (e.g., about 5 kb to about 15 kb (e.g., about 8 kb to about 12 kb)). In one particular example, the nucleic acid comprises about 10 kb.

Chemical transfection of nucleic acids may provide a convenient and robust alternative to viral, liposomal encapsulation and electroporative delivery. Upon complexation with nucleic acids, the physical properties of the resulting nucleic acid complexes may change over time and can affect the functional performance of the complex. In some instance, a transfection complex with a radius of 200-400 nm may be optimal for many applications. Complex formation time may be one of the main parameters that can be varied to control the transfection complex size to achieve nucleic acid delivery. While the functional performance of a transfection complex may not be exclusively due to the size of the complex, dynamic light scattering (DLS) can be used to follow the changing size of the aggregate as it continues to grow over time. Various factors, including the composition of the transfection reagent, salt, and pH can affect the size and functional performance of the resulting complex post addition of nucleic acid.

In some embodiments, optimal complex formation times may be specified for commercial transfection reagent protocols. For example, the recommended TransIT®-mRNA transfection complex formation time is <5 min, while that of TransIT-X2® is 15-30 min. However, in some instances, including, for example, performing a large scale, automated transfection, this time frame may not be preferred or practical since adding and mixing the transfection complex may take longer time. As a result, it is desirable to design a method using a stabilizing polymer-lipid hybrid (PLip) in the transfection reagent formulation prior to complexation with DNA as a way to control or extend the transfection complex formation time.

In some embodiments, optimal complex formation concentration may be a factor for commercial transfection reagent protocols. For example, transfection reagent complexes may be formed at an initial concentration 10× of the concentration of the complex after addition to cells in media (assuming that a 10% vol/vol amount of complex is added compared to the total volume of media in which the cells are transfected). For a 200 L bioreactor, the transfection complex volume to be added would be 20 L for a typical transfection reagent. Adding this volume in a timely manner and maintain the functions of the transfection reagent complexes can be difficult, and lower volumes of the added complex would be preferred (for shorter time of addition and smaller volume to handle). Adding a more concentrated complex would therefore be beneficial, except that complexes grow more quickly and heterogeneously at higher concentration, creating a quality issue (particular in a GMP setting). The addition of a stabilizing PLip may moderate the growth of more concentrated transfection complexes.

### Stimulus-Sensitive Transfection

In an aspect, the lipid polymer compounds provided herein comprise a stimulus-sensitive component in the linker region between the lipid tail and the polymeric headgroup comprising stabilizing monomeric units. These types of stimulus-sensitive, stabilizing PLip not only increase the stability of the transfection reagent complex, but also control the timing the transfection reagents are released in the reactor.

A transfection complex forms when nucleic acids are combined with a transfection reagent (which can include a mixture of polymers and lipids). Through a combination of electrostatic and other noncovalent interactions, cationic, non-viral, non-liposomal formulations may bind the negatively charged nucleic acids to stabilize and condense them. For in vitro delivery, a net positive charge of the complex may enable enhanced cell surface binding. A net positively charged complex can be achieved when the moles of positively charged amines (N) available on the polymer head and/or the lipid tail are greater than the moles of negatively charged phosphates (P) available on the nucleic acid. The N:P ratio is often calculated to determine if a complex can be net positive, neutral, or negative. When incubated with cells, the positively charged complexes may electrostatically interact with the negatively charged cellular membrane, enabling cellular uptake through endocytosis. Once endocytosed, transfection complexes can become trapped in endosomes, potentially leading to the undesired outcomes of lysosomal degradation or cellular export. The PLips disclosed herein not only can stabilize transfection reagents, but also can be stimuli-responsive and can impart a higher net positive charge on the complex upon addition to cells that are maintained at 37° C.

In some embodiments, the physical properties of transfection complexes that evolve during their complex formation step can affect the functional performance of the transfection complexes. These physical properties, such as, for example, size and charge of transfection complexes, can vary depending on several factors, including but not limited to:
Chemical composition of the transfection reagent
Complex formation time of the transfection reagent and nucleic acid
Type and size of nucleic acid being delivered
Media in which the transfection complex is formed in
Molar charge ratio of the transfection reagent to nucleic acid (often reported as the amine/phosphate charge ratio, N/P)
Concentration of the reagent and nucleic acid

Disclosed herein are methods whereby the optimum window for adding the transfection complexes to the cell is widened from minutes to hours (and potentially days) without changing the functional performance of the reagent. The electrostatic interactions between the transfection complex and the cellular components are slowed so that the transfection complex grows much more slowly or is “stabilized” over time. Incorporation of the stabilizing PLip into the transfection complex (which contains a nucleic acid, cationic polymer, and cationic lipid in most cases) occurs via the hydrophobic interactions of its lipid tail. Once incorporated into the complex, the polymeric headgroup of the PLip provides a “stabilizing” moiety. This polymeric headgroup is a sufficiently long, hydrophilic chain that stabilizes the complex by interfering with the electrostatic interactions that are responsible for continued growth of the complexes over time.

Scheme 1 shows example illustrations of the designs for a stabilizing PLip and a stimulus-responsive stabilizing PLip. The difference between the two designs is the insertion of a stimulus-responsive linker unit between the lipid tail and the stabilizing polymeric headgroup in the stimulus-responsive stabilizing PLip. Other designs are possible. For example, multiple stimulus-responsive linker units can be incorporated into the structures of the PLip. For example, one of the monomeric unit of the stabilizing polymeric headgroup may comprise one or more stimulus-responsive linker units. Upon the arrival or application of the stimulus, the stimulus-responsive linker unit may change its chemical and/or physical properties, thereby impacting the functions of the transfection complex. The stimulus can be heat, light, chemical, pH, etc., to trigger the change of the properties of the Plips. The changes may be from an extended form or coil form to a condensed form or a globule aggregate, from positively charged to neutral or negatively charged, from neutral to negatively charged, from neutral to positively charged, from negatively charged to neutral or positively charged, from stable to unstable, etc.

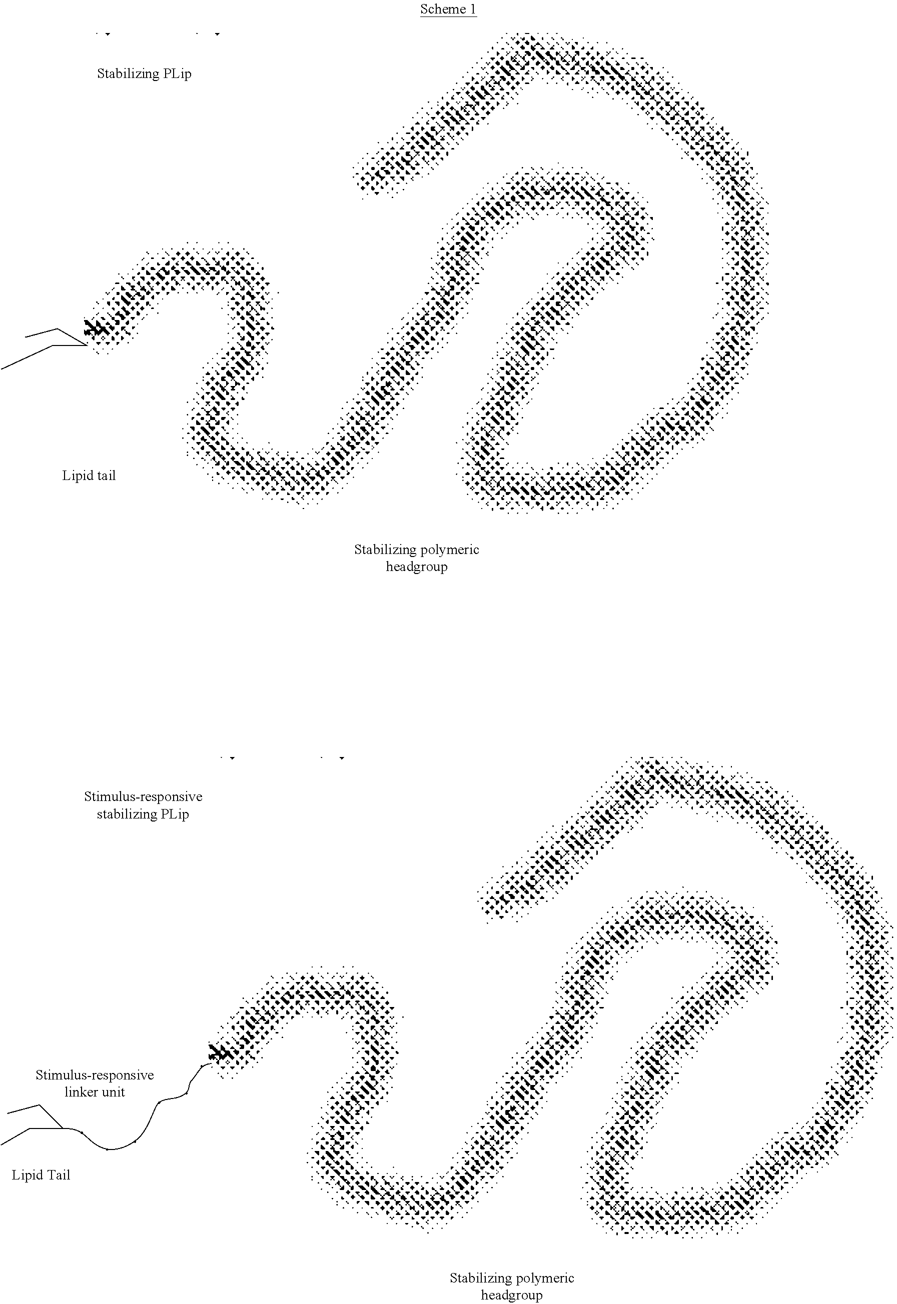

### Temperature-Responsive PLips

For in vitro transfection complexes in particular, the zeta potential and overall positive charge of the particles may impact the delivery to the surface of the negatively charged cell membrane surface. Therefore, the properties and concentration of the stabilizing PLip can play a role. For instance, on the one hand, adding too much stabilizing PLip may prevent the electrostatic interactions with other transfection complexes as well as electrostatic interactions with the cells. On the other hand, adding too little PLip may not have the desired effect of stabilizing the complex and elongating the optimum time allowed for optimal size and transfection efficiency. Similarly, PLips with headgroups that are too long or too short may have similar effects.

In some embodiments, the PLip may be stimuli-responsive. In some embodiments, the temperature-sensitive PLips may contain, (i) at one end, a lipid tail end group (for interacting with the transfection complex), (ii) in the middle, a temperature sensitive linker unit group comprising a polymer section (for example: poly(N-isopropylacrylamide) (“P(NIPAm)”)), and (ii) at the other end, a stabilizing polymeric headgroup comprising another polymer section (for example: PEG, poly(2-hydroxyethyl acrylate) (“PHEA”) or poly[oligo(ethylene glycol) methyl ether methacrylate](“POEGMA”)). The use of a temperature sensitive PLip can exploit the fact that transfection complexes can be formed at room temperature (25° C.) or below, while the transfection of cells is performed at 37° C. Some polymers, such as P(NIPAm), may have a lower crystalline solution temperature (LCST) of 32° C. or lower in water, meaning it undergoes a transition from being soluble (hydrophilic-like) in water below this temperature to insoluble (“hydrophobic”-like) above this temperature. If above the LCST, the P(NIPAm) polymer chains may not adopt an extended, random coil structure since protons on the P(NIPAm) side chain no longer form hydrogen-bonds with the surrounding water molecules, and, instead, interact with protons from other P(NIPAm) side chains. This transition may cause the P(NIPAm) to adopt a collapsed structure and exclude the water molecules. When incorporated into a transfection complex, the net effect can be a complex that is more stable and less cationic at room temperature, and less stable and more cationic at 37° C. Scheme 2 below show a schematic drawing of the stabilizing hydrodynamic volumes associated with the transfection complexes at 25° C. and 37° C.

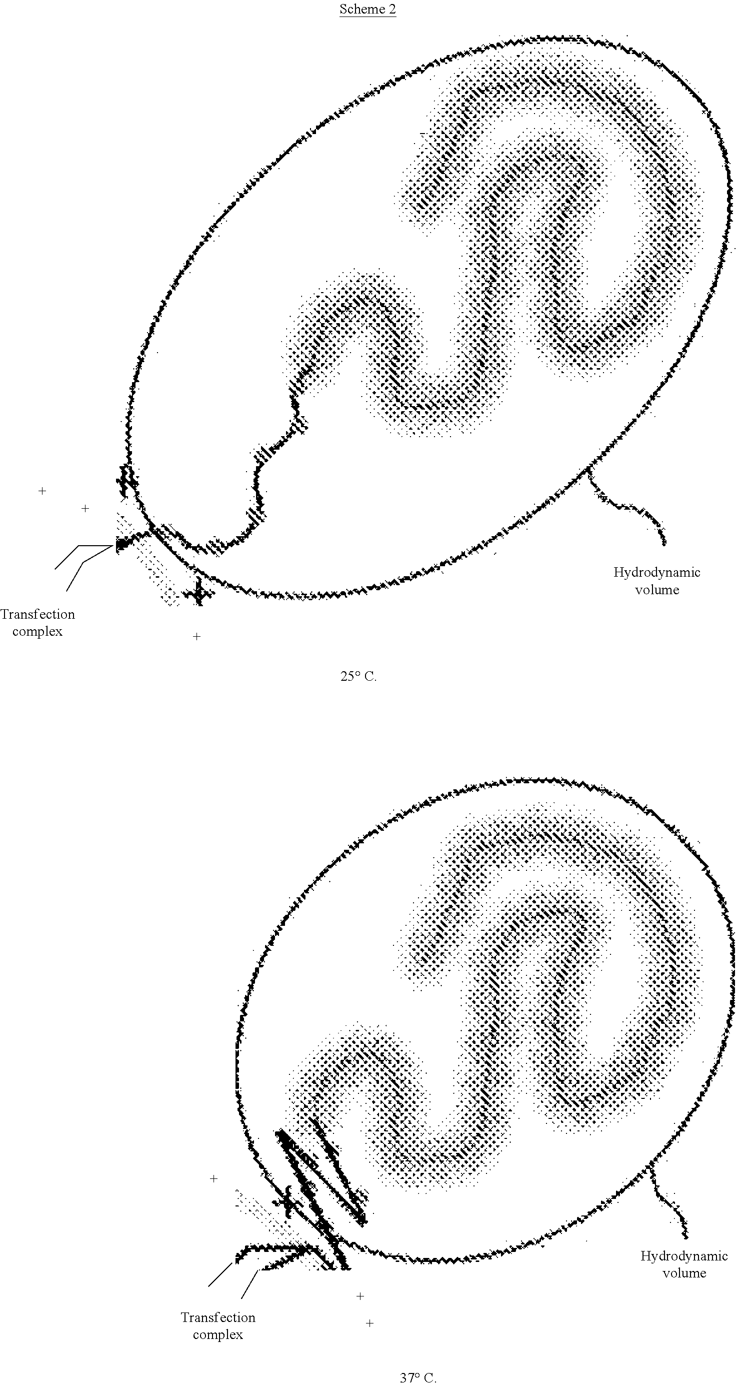

At 25° C. the hydrodynamic volume may be larger, and the stability of the transfection complex may be higher when compared to 37° C. These properties may be the results of the extended P(NIPAm) linker unit at 25° C. and the presence of the POEGMA unit in the polymeric headgroup. At 37° C. the P(NIPAm) block has collapsed and is no longer hydrophilic, leaving only the outer POEGMA block as a stabilizing moiety.

In some embodiments, the temperature-responsive unit comprises a lower crystalline solution temperature (LCST) from about 27° C. to about 35° C. In some embodiments, the temperature-responsive unit comprises a lower crystalline solution temperature (LCST) at about 27° C., about 28° C., about 29° C., about 30° C., about 31° C., about 32° C., about 33° C., about 34° C., or about 35° C. In some embodiments, the temperature-responsive unit comprises poly(N-isopropylacrylamide), poly(N-n-propylacrylamide), poly(N-methyl-N-n-propylacrylamide), poly(N,N-diethylacrylamide), poly(N-isobutylacrylamide), poly(N-sec-butylacrylamide), poly(N-n-butylacrylamide), poly(N-isobutylacrylamide), hydroxypropylcellulose, poly(N-vinylcaprolactam), poly-2-isopropyl-2-oxazoline, or polyvinyl methyl ether, or a combination thereof. In some embodiments, the temperature-responsive unit comprises 2-250 monomeric units.

Other examples of stimuli-responsiveness might be pH-responsive Plips. In some embodiments, the pH-responsive Plips may stabilize a transfection complex at pH 7.4, but may become less stabilizing at lower pH, such as the pH encountered in the endosome or other cells. The pH-responsive Plips may swell, collapse, or change its structure or shape depending on the pH of their environment. This pH dependent behavior may be exhibited due to the presence of certain pH sensitive functional groups in the polymer chain. The sensitivity can be either acidic liable or basic liable, responding to either basic or acidic pH conditions as stimulus. For example, polymers with acidic groups (such as —COOH and —SO₃H) and polymers with basic groups (—NH₂) can be pH-sensitive polymers. The mechanism of response may be similar for both groups, only the stimulus varies.

Examples of polyacid polymers (anionic polymers) comprise acidic functional groups including carboxylic acids (—COOH), sulfonic acids (—SO₃H), phosphonic acids, and boronic acids. Accordingly, Polyacids may accept protons at low pH values. At higher pH values, they may deprotonate and become negatively charged. The negative charges may create a repulsion that causes the polymer to swell. This swelling behavior may be observed when the pH is greater than the pKa of the polymer. Examples of polyacid polymers may include polymethyl methacrylate polymers and cellulose acetate phthalate. In some instance, poly(methacrylic acid) (PMAAc) may accept protons at low pH and release protons at neutral and high pH. Additional examples of polyacid polymers may include: poly(carboxylic acid), poly(phosphoric acid), poly(sulfonic acid), poly(amino acid), and poly(boronic acid).

Examples of polybase polymers may be the basic equivalent of polyacid polymers and may also be known as cationic polymers. They may accept protons at low pH like polyacid polymers do, but they may then become positively charged. In contrast, at higher pH values they are neutral. Swelling behavior may be seen when the pH is less than the pKa of the polymer. Poly[(2-dimethylamino)ethyl methacrylate] (PDMA) may accept protons at low pH and thus form a positively charged polymer chain. Other examples may include polymers containing tertiary amine group, morpholino group, pyrrolidine group, piperazine group, pyridine group, imidazole group as part of the side chains of the polymers.

Other pH-sensitive polymers may include alginic acid, chitosan, carboxymethyl cellulose, carboxymethyl dextran, gelatine A and B, and hyaluronic acid.

### Methods of Use

Disclosed herein are methods for transfecting a cell, wherein the methods comprise (a) providing a transfection reagent comprising the compound disclosed herein and a nucleic acid, and (b) contacting the cell with the transfection reagent, wherein the contacting is under conditions suitable for entry of the nucleic acid into the cell. In some embodiments, step (a) comprises contacting said compound with said nucleic acid under conditions sufficient to form said transfection complex. In some embodiments, in steps (a) and (b), the conditions sufficient to form the transfection complex comprise conditions sufficient for ionotropic gelation.

In some embodiments, provided herein are methods for transfecting a cell, wherein the transfection complex comprises a positive charge under the conditions suitable for entry of the nucleic acid into the cell.

In some embodiments, provided herein are methods for transfecting a cell, wherein the nucleic acid comprises deoxyribonucleic acid (DNA), ribonucleic acid (RNA), locked nucleic (LNA), peptide nucleic acid (PNA), or any combination thereof.

The present invention provides methods for transfecting a cell, wherein the contacting is for less than about 24 hours. In some embodiments, the contacting is for less than about 20 hours, less than about 18 hours, less than about 16 hours, less than about 14 hours, less than about 12 hours, less than about 10 hours, less than about 8 hours, less than about 6 hours, less than about 4 hours, less than about 2 hours, or less than about 1 hour. In some embodiments, the cell comprises an animal cell, a plant cell, a fungal cell, a bacterial cell, or any combination thereof.

In some embodiments, provided herein is a method of performing lipid-mediated transfection of a cell comprising contacting the cell with a transfection reagent disclosed herein (e.g., a composition comprising a compound described herein and a nucleic acid). In some embodiments, provided herein are methods of preparing a compound (e.g., a lipid-polymer conjugate) or a transfection reagent comprising the same, as described in the following examples.

### ABBREVIATIONS

Except where otherwise stated, abbreviations used throughout this disclosure are defined as follows:
¹H NMR proton nuclear magnetic resonance
AIBN 2,2′-azobis(2-methylpropionitrile)
AF488 azidefluor-488
C Celsius
CPCPA 4-cyano-4-((phenylcarbonothioyl)thio)pentanoic acid
DBCO dibenzocyclooctyne
DCM dichloromethane
DIC diisopropylcarbodiimide
DLIN (6Z,9Z,28Z,31Z)-heptatriacont-6,9,28,31-tetraene-19-yl 4-(dimethylamino) butanoate or DLin-MC3-DMA
DLS dynamic light scattering
DMAP 4-dimethylaminopyridine
DMF N,N-dimethylformamide
DNA deoxyribonucleic acid
DOPE dioleoyl phosphatidylethanolamine
EtOH ethanol
HEA hydroxyethylacrylate
HPLC high pressure liquid chromatography
HPMA N-(2-hydroxypropyl)methacrylamide
LC/MS liquid chromatography/mass spectrometry
LNP lipid nanoparticle
MeOH methanol
MFI mean fluorescence intensity
MSEMA 2-(methylsulfinyl)ethyl methacrylate
NI normalized intensity
NIPAm N-isopropylacrylamide
PEG-DMG 1,2-dimyristoyl-rac-glycero-3-methoxypolyethylene glycol
P(HPMA) poly(N-(2-hydroxypropyl)methacrylamide)
PLIP polymer-lipid compound
RAFT reversible addition-fragmentation chain-transfer polymerization
RFU relative fluorescence units
SMFI specific mean fluorescence intensity
TLC thin-layer chromatography
v/v volume by volume ratio

### EXAMPLES

The following illustrative examples are representative of embodiments of the compounds, compositions, and methods described herein and are not meant to be limiting in any way.

### SYNTHESIS EXAMPLES

Compounds disclosed herein can be synthesized according to, or in analogy to, the following synthesis examples. Furthermore, the examples disclosed herein can be adapted and modified according to principles known in the art to produce a variety of polymer-lipid compounds, synthetic intermediates (e.g., RAFT agents), and transfection reagents (e.g., LNPs) as disclosed herein.

### Example S1: Synthesis and Characterization of a Dioleyl Raft Agent

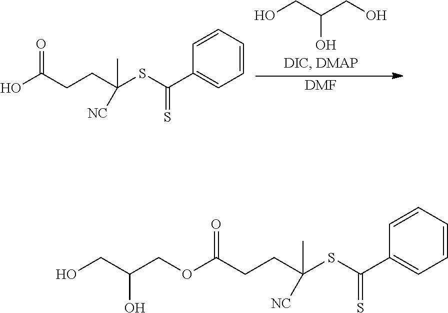

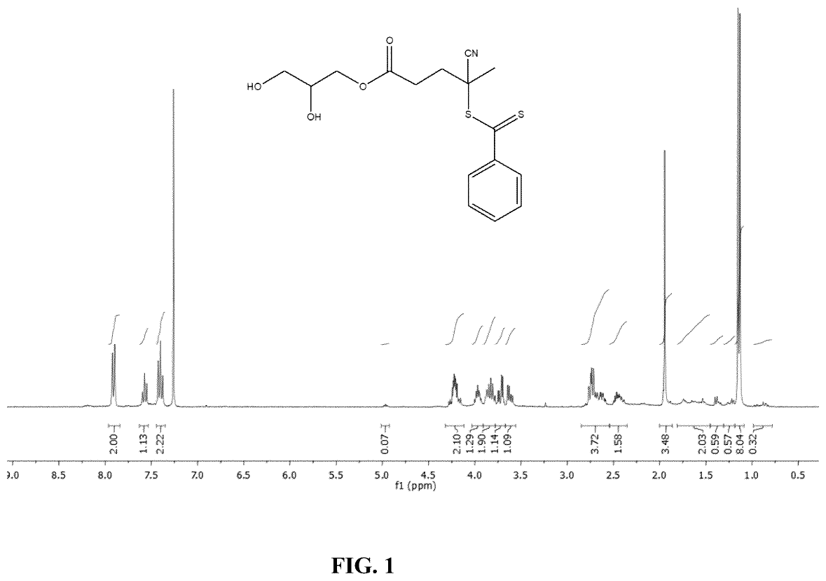

i. To a sample of 4-cyano-4-((phenylcarbonothioyl)thio)pentanoic acid (“CPCPA”) (4.00 g, 14.3 mmol) in DMF (20 mL) was added an excess of glycerol (16.0 g, 0.118 mol) and diisopropylcarbodiimide (DIC, 2.80 mL, 17.9 mmol), stirring at room temperature for 30 min. Next, 4-dimethylaminopyridine (DMAP, 78.0 mg, 0.64 mmol) was added and the mixture was left to stir overnight. Ethyl acetate (40 mL) was added and the resulting mixture was extracted with water (40 mL) to remove the DMF. The aqueous layer was extracted with ethyl acetate (15 mL) and combined with the organic layers. The organic layers were then re-extracted with water (40 mL). The organic solution was dried via rotary evaporator, and the resulting oil was dissolved in methanol (12.5 mL). The methanol solution was then precipitated by dropping into water (80 mL) twice. To the precipitate was added chloroform (40 mL), and the solution was dried over sodium sulfate before filtering through filter paper. The solution was dried via rotary evaporator and redissolved in chloroform (10 mL). The chloroform solution was then precipitated twice into hexane (80 mL) then dried under reduced pressure. The CPCPA-diol product was characterized by TLC (chloroform:methanol 90:10%, Rf=0.23) and ¹H NMR spectroscopy (see FIG. 1). Yield 3.60 g.

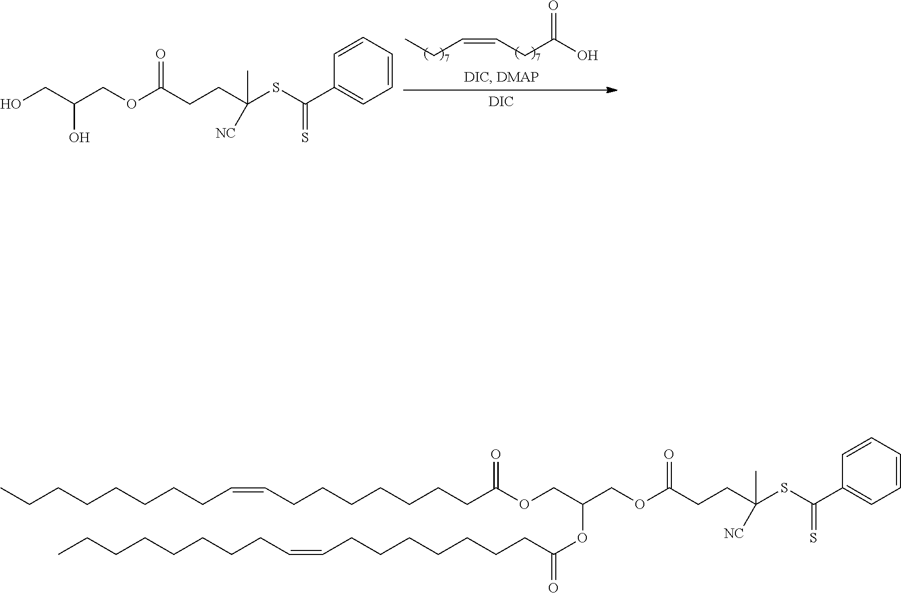

ii. The CPCPA-diol product of Step 1 (0.500 g, 1.42 mmol) was dissolved in DCM (15 mL). Oleic acid (1.20 g, 4.25 mmol) and DIC (0.665 mL, 4.25 mmol) were added and stirred at room temperature for 30 min. Then, DMAP (26.5 mg, 0.217 mmol) was added and the mixture stirred for 2 h. The solution was extracted with water (15 mL) twice before drying the organic layer with sodium sulfate. The solution was filtered through fluted filter paper and dried via rotary evaporation. The resulting mixture was dissolved in chloroform (2 mL) and precipitated into a 9:1 MeOH/H₂O (v/v) solution (40 mL) twice. The product was dissolved in chloroform (10 mL) and dried over sodium sulfate, then filtered and dried in vacuo. The product was characterized by TLC (chloroform:methanol 95:5%, Rf=0.87) and ¹H NMR spectroscopy.

### Example S2: Synthesis and Characterization of a Dilinoleyl Raft Agent

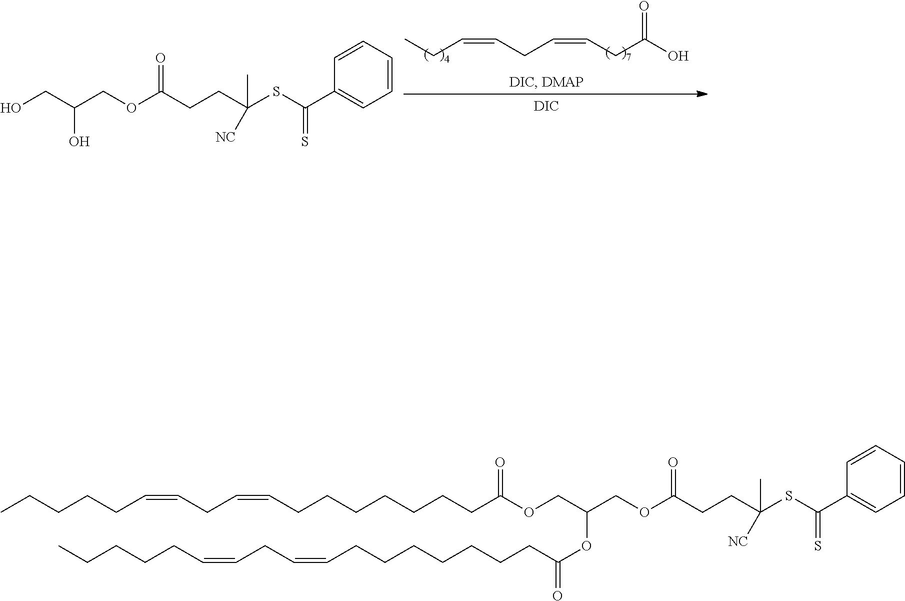

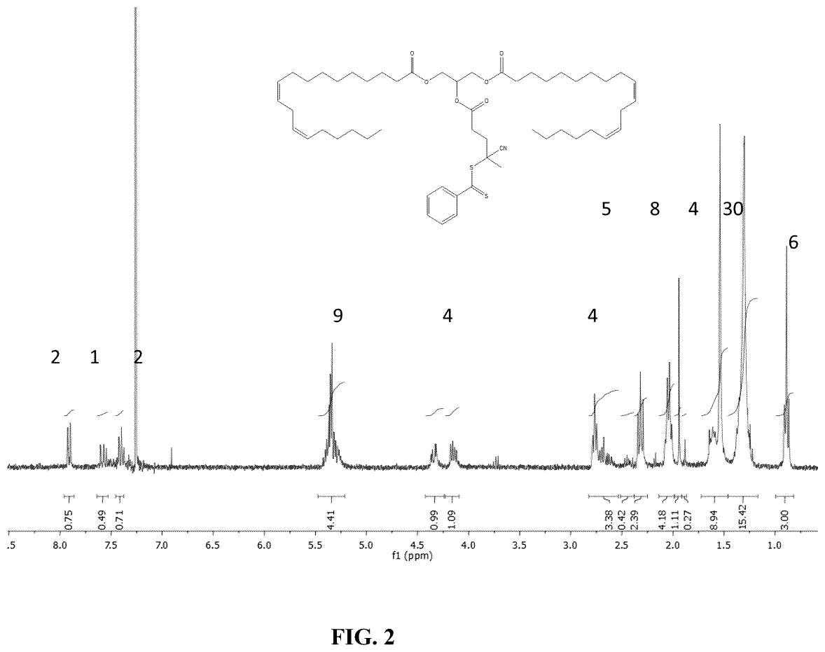

The CPCPA-diol product from step (i) of Example S1 (0.500 g, 1.42 mmol) was dissolved in DCM (15 mL). Linoleic acid (1.19 g, 4.25 mmol) and DIC (0.665 mL, 4.25 mmol) were added and stirred at room temperature for 30 min. Then, DMAP (26.5 mg, 0.217 mmol) was added and the mixture stirred for 2 h. The solution was extracted with water (15 mL) twice before drying the organic layer with sodium sulfate. The solution was filtered through fluted filter paper and dried via rotary evaporation. The concentrate was dissolved in chloroform (2 mL) and precipitated into a 9:1 MeOH/H₂O (v/v) solution (40 mL) twice. The precipitate was collected and dissolved in chloroform (10 mL), dried over sodium sulfate, then filtered and dried in vacuo. The product was characterized by TLC (chloroform:methanol 95:5%, Rf=0.87) and ¹H NMR spectroscopy, as shown in FIG. 2.

### Example S3: Synthesis and Characterization of a Cholesterol Raft Agent

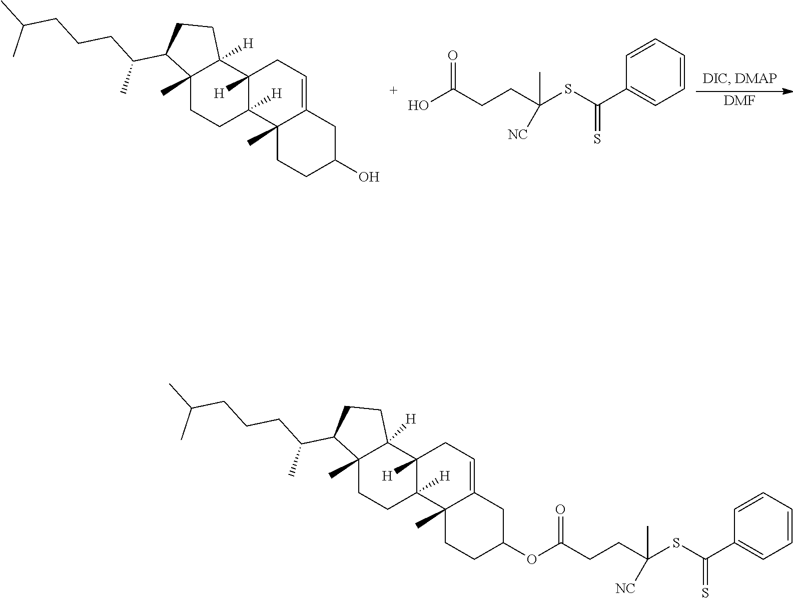

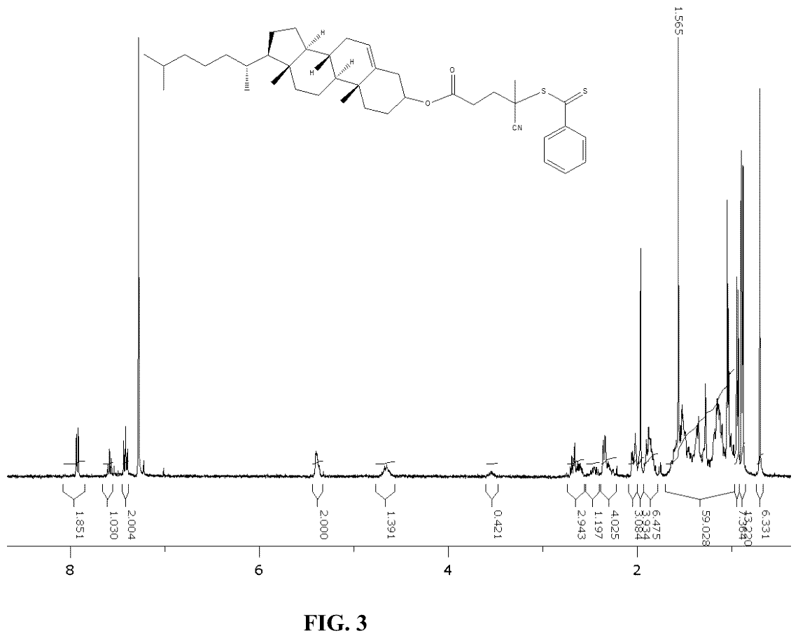

To a sample of CPCPA (1.00 g, 3.58 mmol) in DCM (15 mL) was added cholesterol (1.94 g, 5.01 mmol) and DIC (2.80 mL, 17.9 mmol), stirring at room temperature for 30 min. Next, DMAP (31.6 mg, 0.259 mmol) was added and the mixture was left to stir overnight. The solution was extracted with water (30 mL) twice before drying the organic layer with sodium sulfate. The solution was filtered through fluted filter paper and dried via rotary evaporation. The concentrate was dissolved in DCM (3 mL) and precipitated into a 9:1 MeOH/H₂O (v/v) solution (45 mL) twice. The mixture was spun down to separate the oil before dissolving in DCM (3 mL) and precipitating in MeOH/H₂O once more. The precipitate was collected and dissolved in DCM (10 mL), dried over sodium sulfate, then filtered and dried in vacuo. The product was characterized by TLC (chloroform:methanol 95:5%, Rf=0.62) and ¹H NMR spectroscopy, as shown in FIG. 3.

### Example S4: Synthesis of Additional Diacyl Raft Agents

The following diacyl RAFT agents were synthesized in analogy to Examples S1-S3:

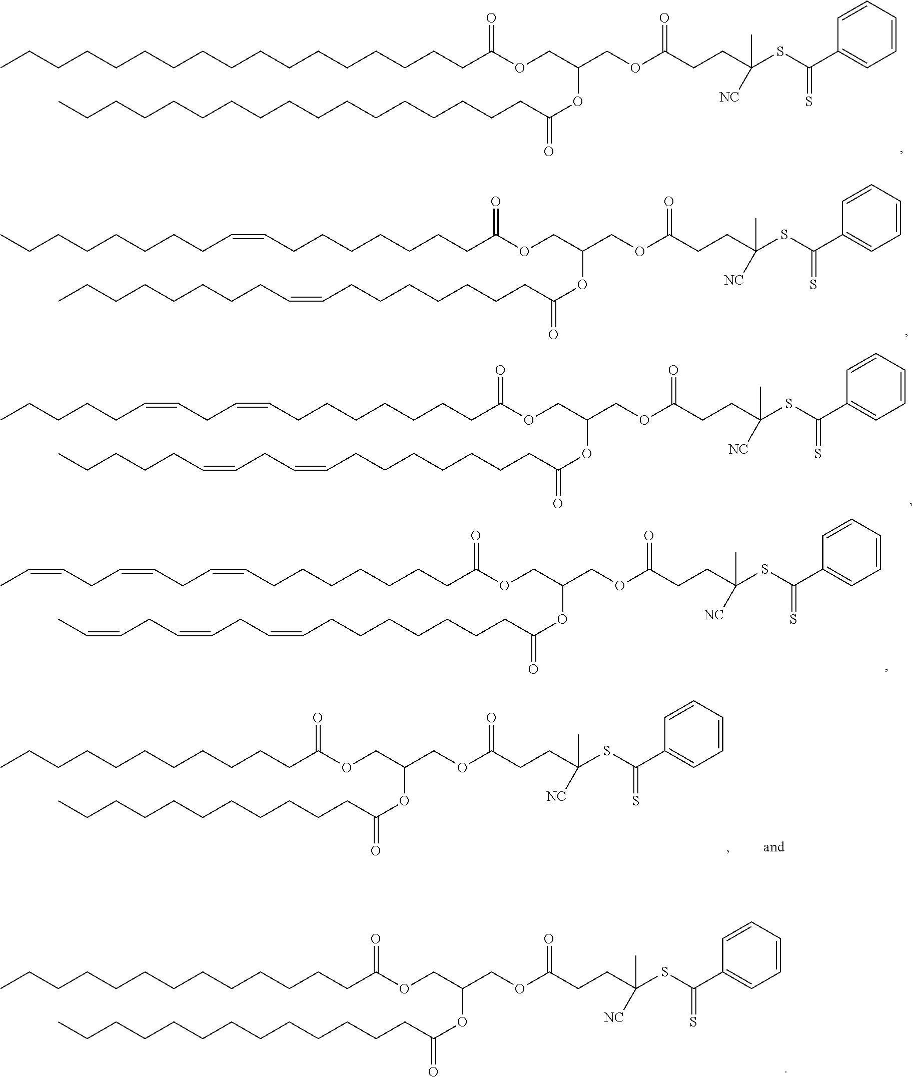

### Example S5: Synthesis of Monoacyl Raft Agents

The following monoacyl RAFT agents were synthesized in analogy to Examples S1-S3:

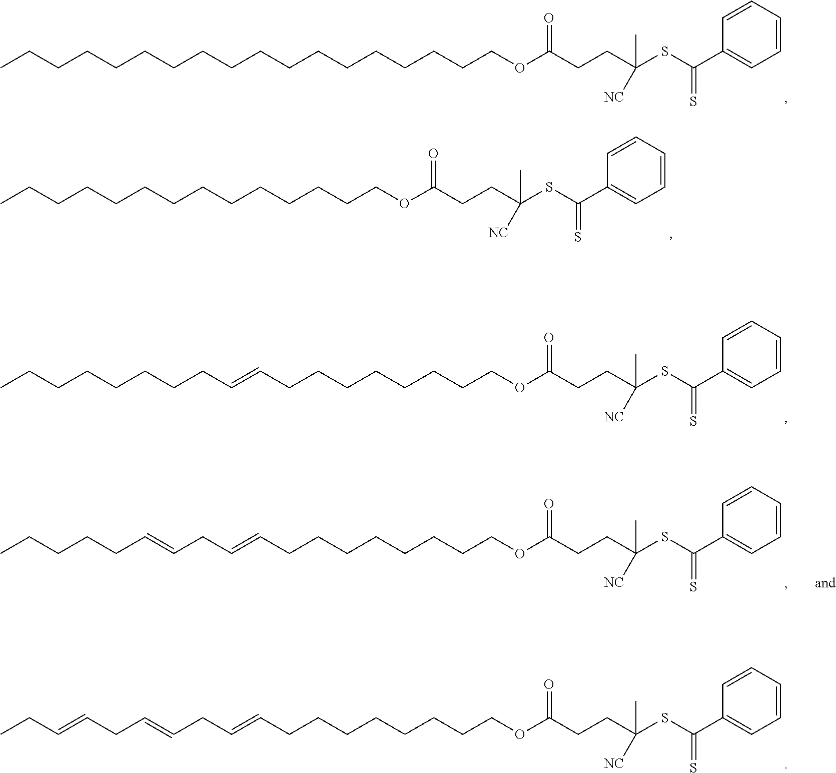

### Example S6: Synthesis of Trithiocarbonate Raft Agents

The trithiocarbonate RAFT agents below were synthesized in analogy to Examples S1-S3:

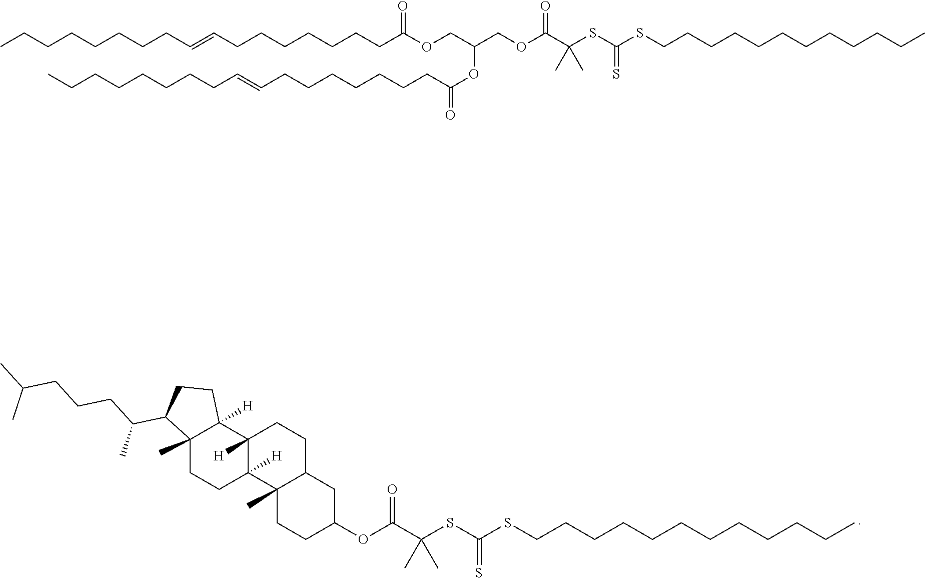

### Example S7: Synthesis of Polymer-Lipid Compounds (“PLIPS”)

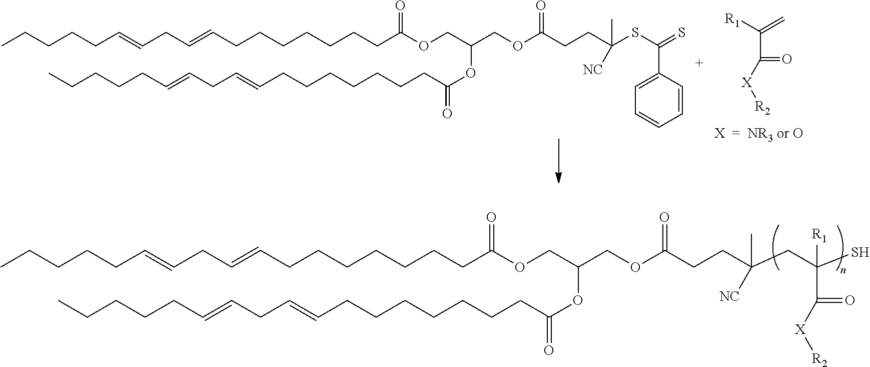

Polymer-lipid compounds (PLips) can be synthesized using reversible addition-fragmentation chain-transfer polymerization (RAFT), using an acrylate or acrylamide, and any one of the RAFT agents disclosed herein (e.g., in Examples S1-S6), in combination with a radical initiator (e.g., 2,2′-Azobis(2-methylpropionitrile), “AIBN”). The monomer(s) and RAFT agent are heated in the presence of an initiator under inert atmosphere using a suitable solvent, e.g., dioxane. A grafting from polymerization process results in the growth of a polymer chain with lipid tails situated at the a-end of the polymer. More than one monomer can be incorporated into the chain to form random/statistical copolymers (monomers added together) or block/gradient copolymers (monomers added sequentially).

### Example S8: Synthesis of Cationic PLips

### (a): Synthesis of a Cationic Diacyl PLip

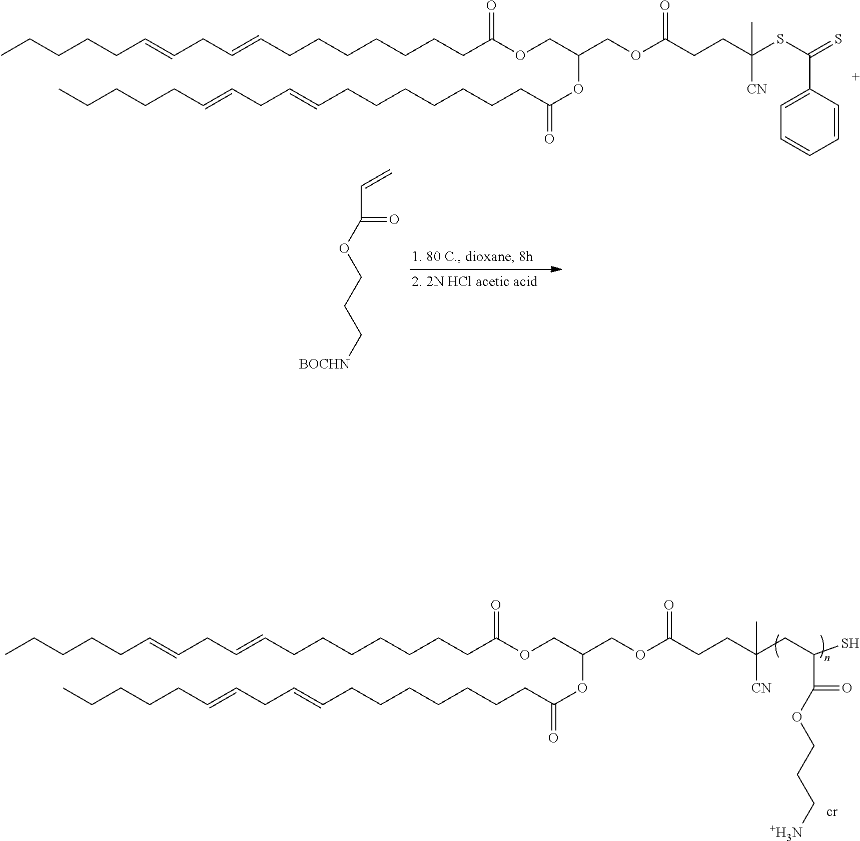

i. The CPCPA-diacyl RAFT agent starting material was prepared according to the general procedures disclosed in Examples S2 and S3, starting from the CPCPA-diol of Example S1.

ii. A 10 mg/mL solution of ABN in dioxane was prepared for use in the polymerization reaction. To a 2 mL sealed glass vial with septum cap was added Boc-aminopropyl acrylate (55.0 mg, 0.241 mmol), the RAFT agent of Scheme S8 (24.1 mg, 0.0275 mmol), AIBN solution (0.0677 mL, 0.00413 mmol AIBN), and dioxane (0.37 mL). The flask was sealed and the reaction solution sparged with nitrogen for 20 min before heating at 80° C. for 8 h while stirring. Upon cooling to room temperature, the solution was precipitated into hexane (14 mL), using DCM (0.2 mL) to transfer the solution. Precipitation was repeated again into hexane (14 mL) from 0.5 mL of DCM. The precipitate was then dried in vacuo and analyzed by ¹H NMR and GPC analysis. Mn 7,100 (PDI 1.26). Yield=35-50 mg (45-65%).

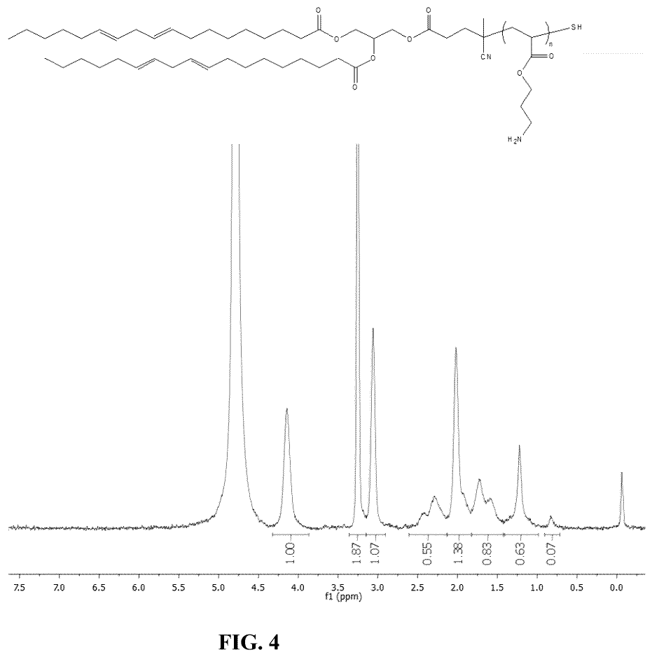

iii. The dried precipitate of step (ii) (40 mg) was dissolved in a 2N HCl in acetic acid solution (1.0 mL) and stirred for 1 h. Deionized water (2 mL) was added to the solution, which was then placed in dialysis bags (MWCO 1,000), dialyzed against salt water, then deionized water. The solution was then removed from the dialysis bags and lyophilized to dryness. The cationic PLip was characterized by ¹H NMR (FIG. 4).

iv. A 20 mg/mL solution was prepared by dissolving the lyophilized PLip from the preceding step (iii) in a 1:1 mixture of EtOH/H₂O. The solution was stored at 4° C. Yield=10-15 mg (35-60%).

### (b): Synthesis of a Cationic Sterol PLip

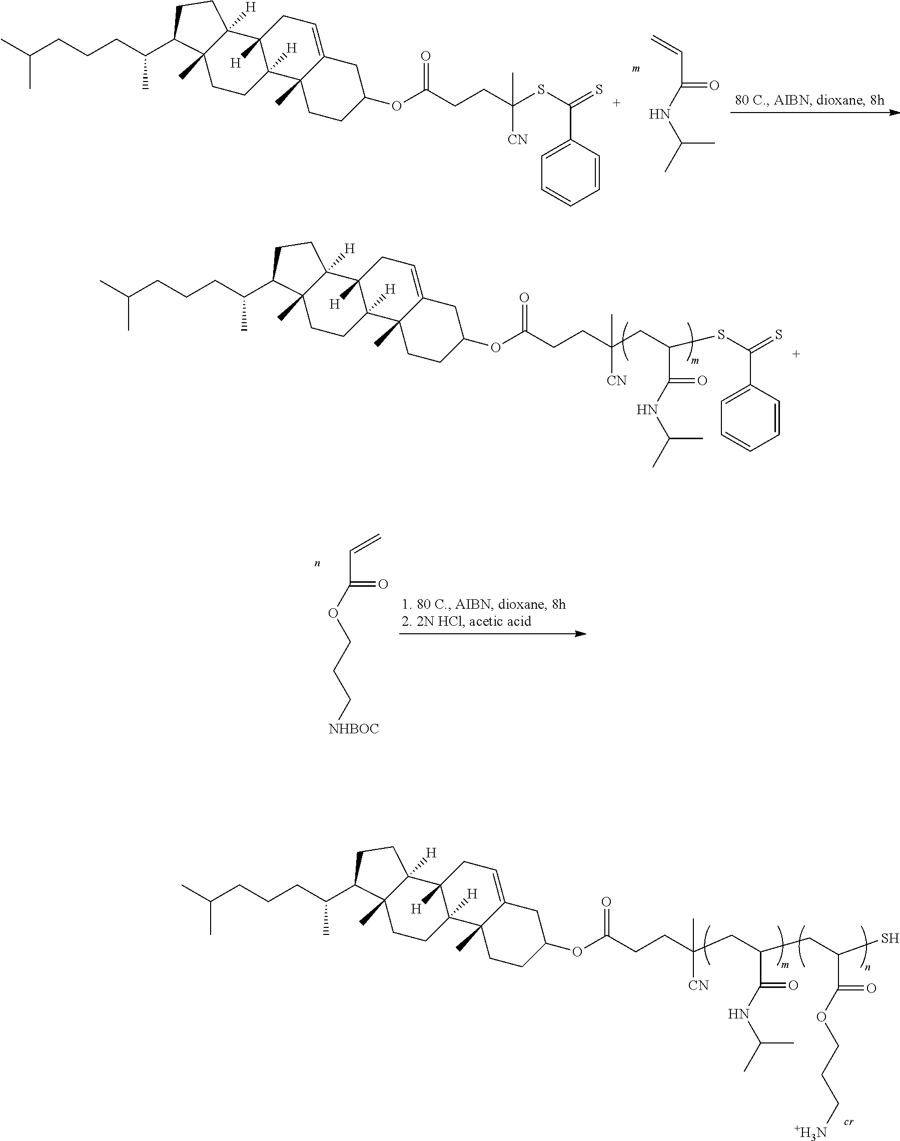

The cationic cholesterol PLip was synthesized according to Example S8(a), replacing the diacyl RAFT agent with the cholesterol RAFT agent of Example S3. The subsequent isolation, acidification, dialysis, and formulation steps were carried out analogous to Example S3 steps (ii-iv).

### Example S9: Synthesis of Non-Cationic PLips

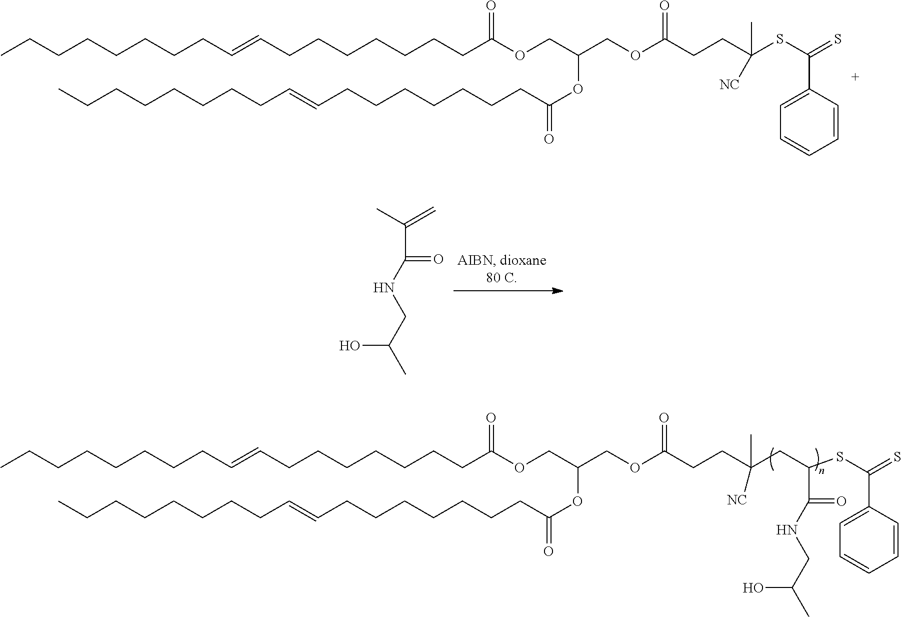

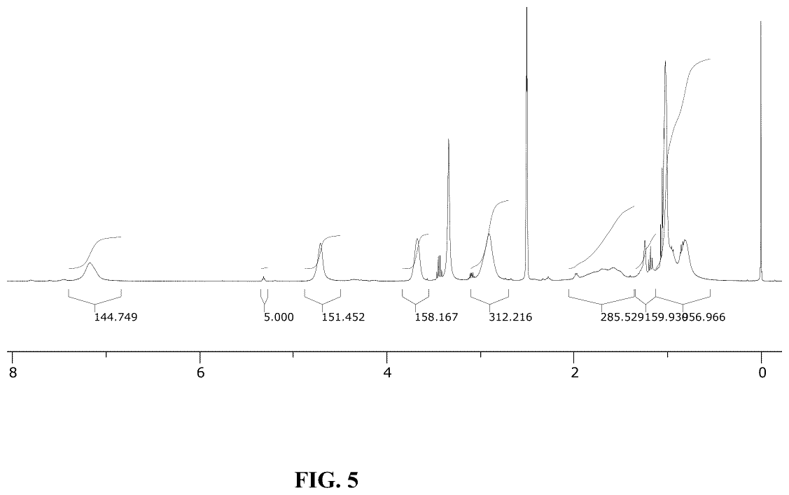

i. N-(2-hydroxypropyl)methacrylamide (HPMA) (1.05 g, 7.36 mmol), CPCPA-diacyl RAFT agent (86.7 mg, 0.0981 mmol), AIBN solution (3.2 mg, 0.0196 mmol AIBN), and dioxane (7.36 mL) were combined in a 20 mL glass vial, which was then sparged with nitrogen for 40 min before heating at 80° C. while stirring. After 8 h, the solution was cooled to room temperature, and the PLip was precipitated into hexane (90 mL) in triplicate, using ethanol as a transfer solvent (5 mL). The precipitate was dried in vacuo and analyzed by ¹H NMR (FIG. 5) and GPC analysis. Mₙ 11,200. Yield=801 mg (70%).

ii. A 20 mg/mL solution was prepared by dissolving the dried PLip from the preceding step (i) in a 1:1 mixture of EtOH/H₂O. The solution was stored at 4° C.

### Example S10: Additional PLips

The following PLips were prepared according to the preceding examples S1-S9:

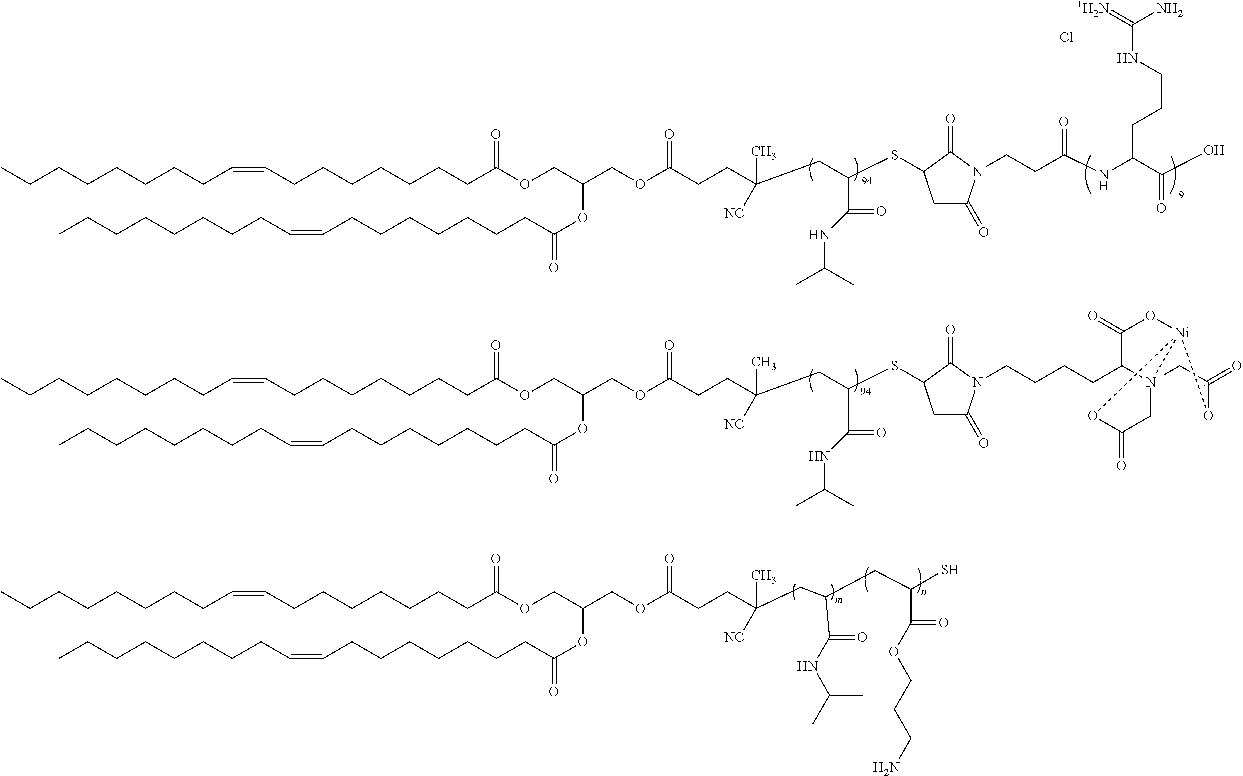

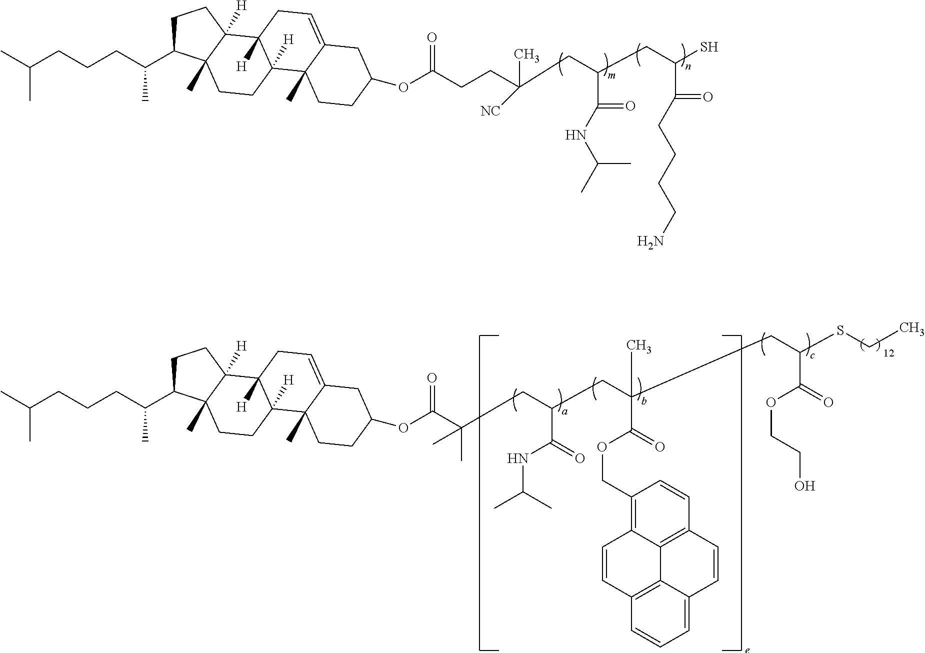

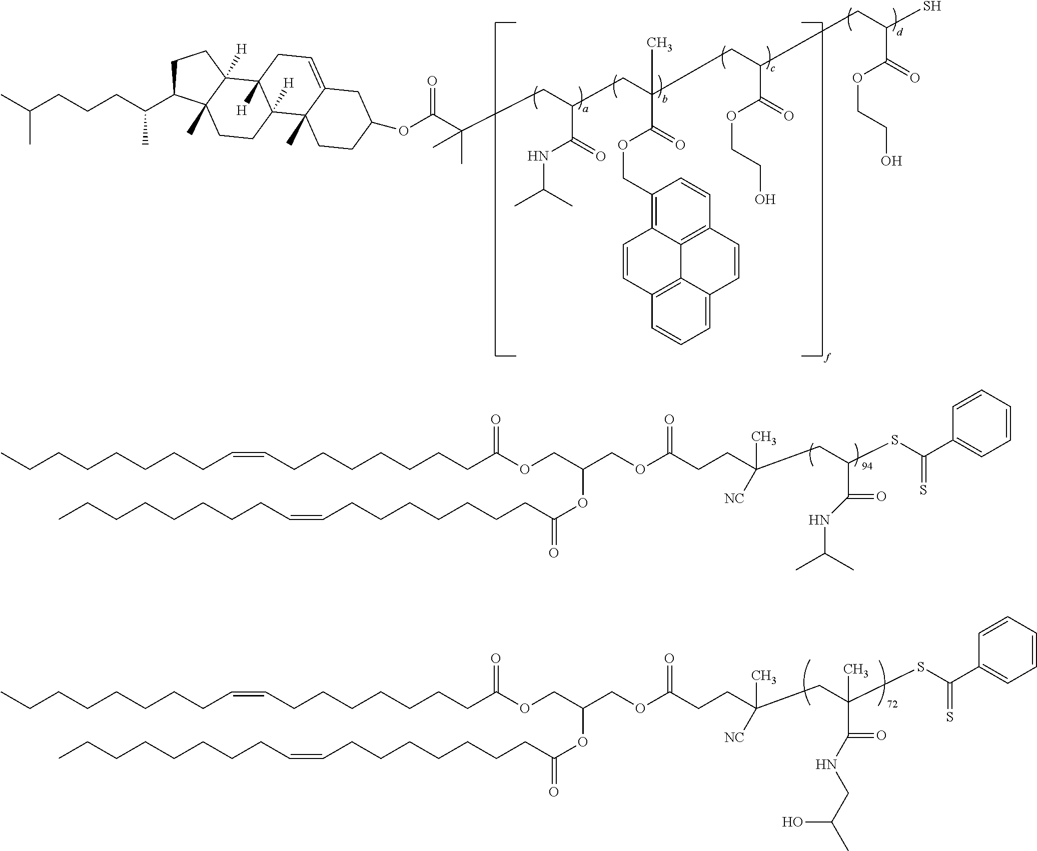

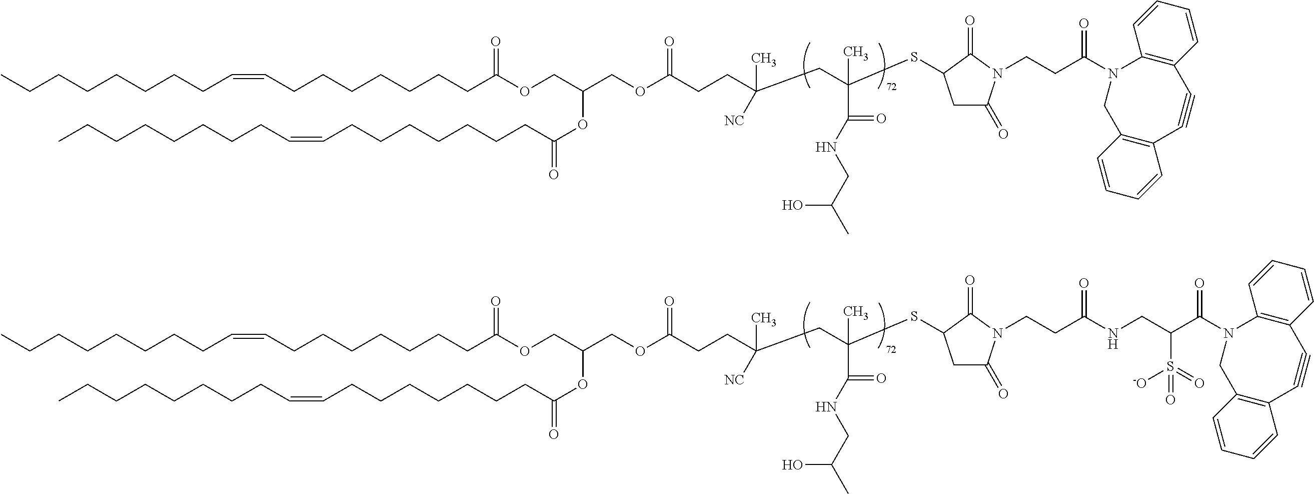

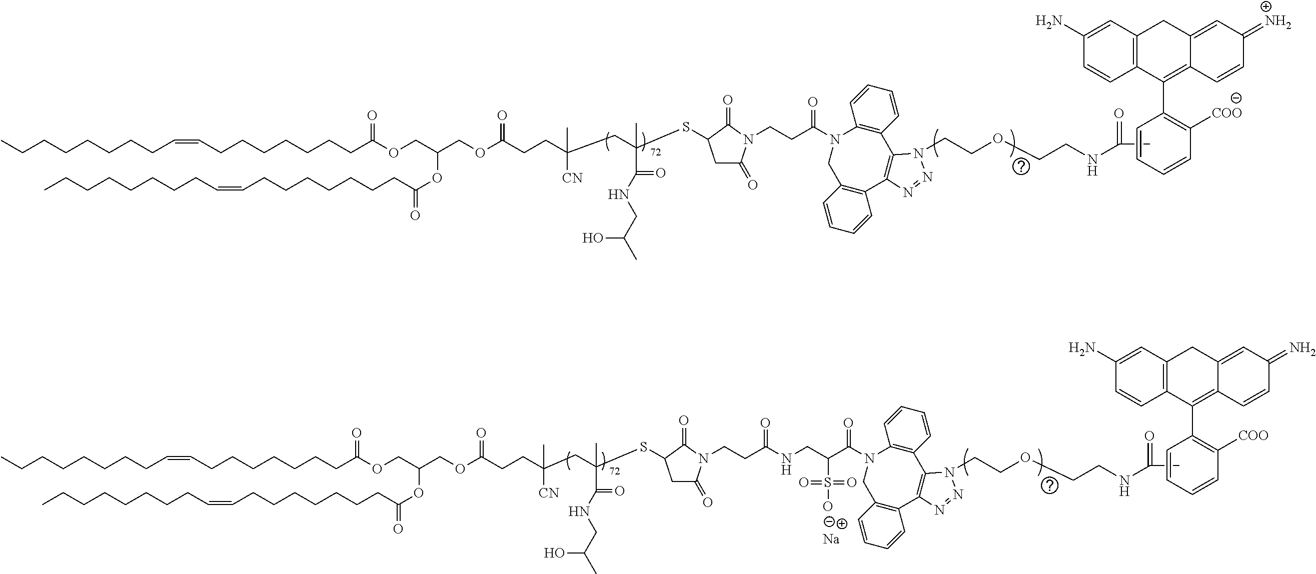

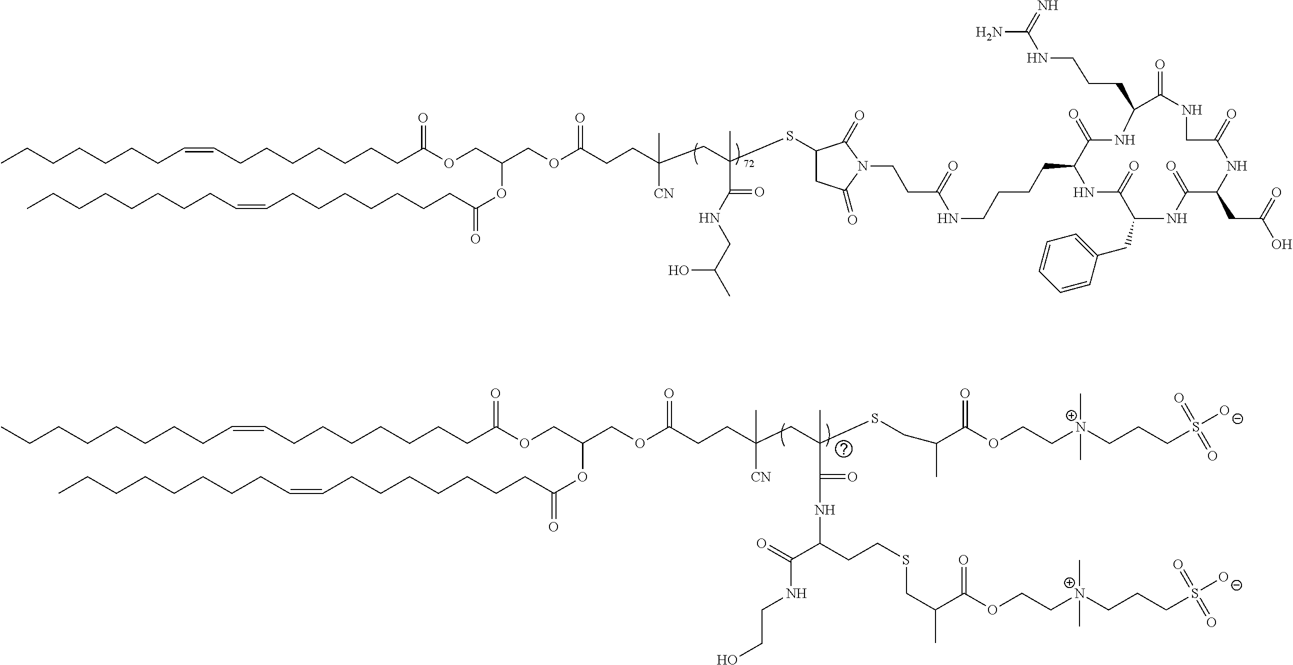

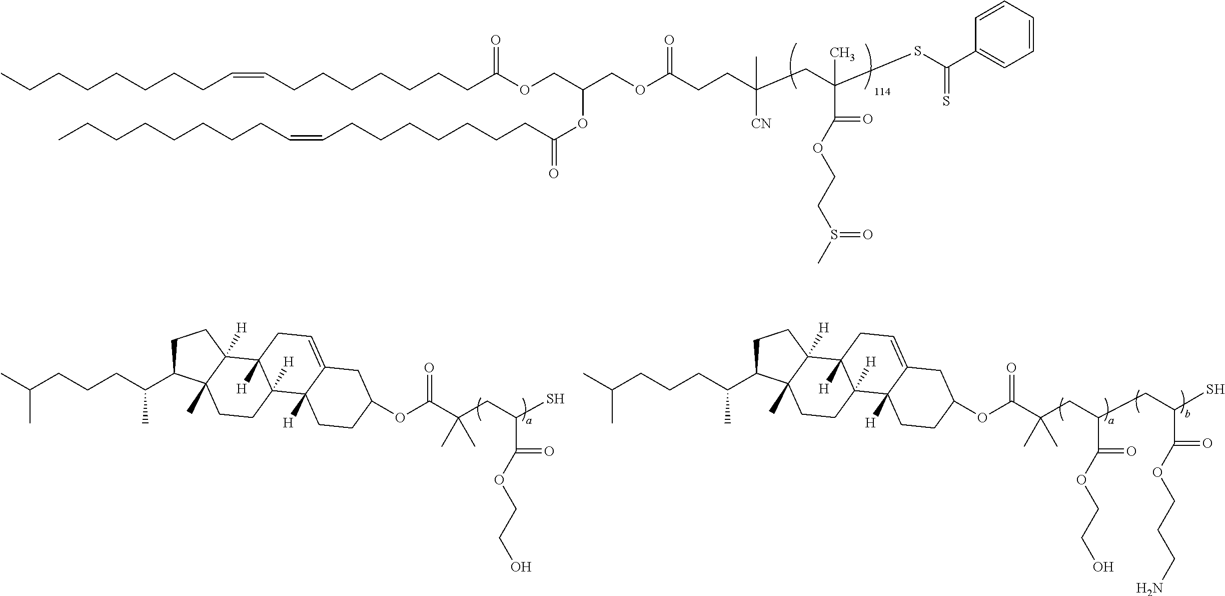

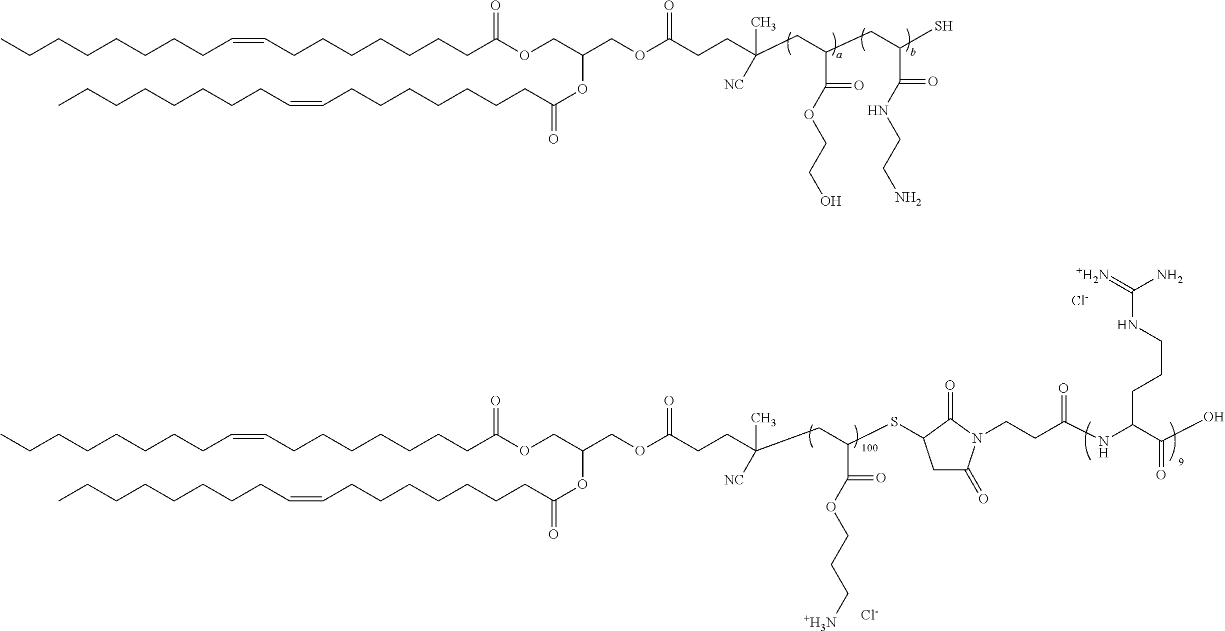

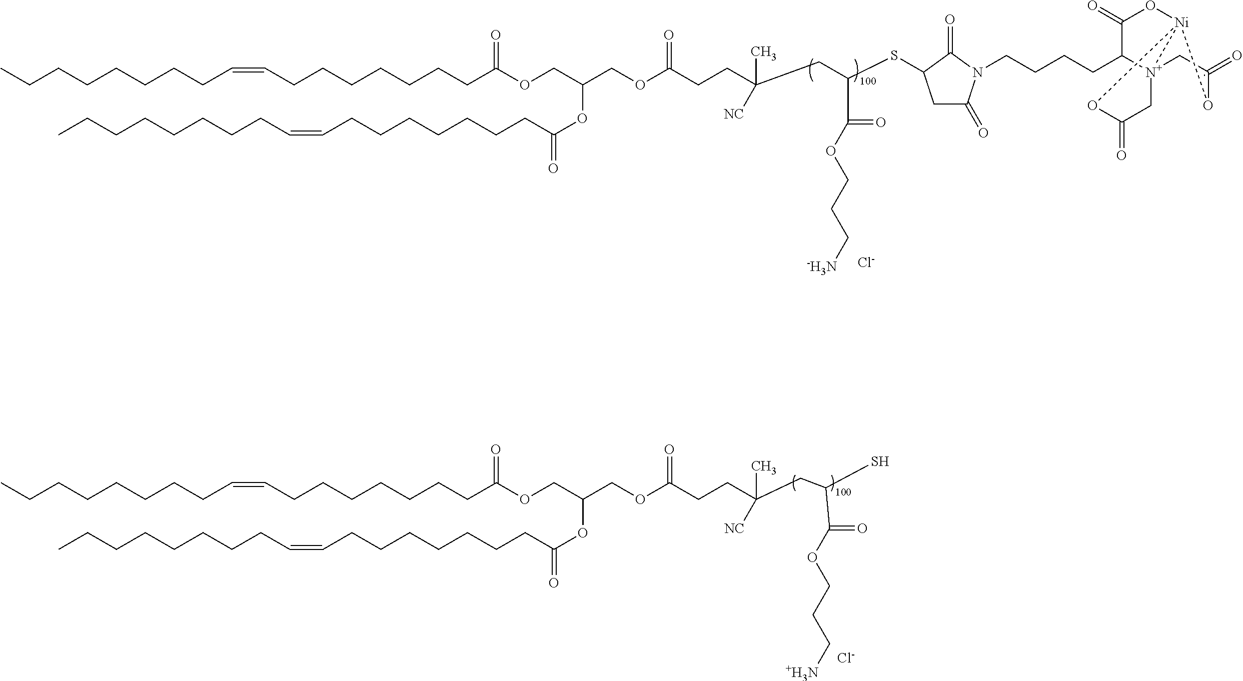

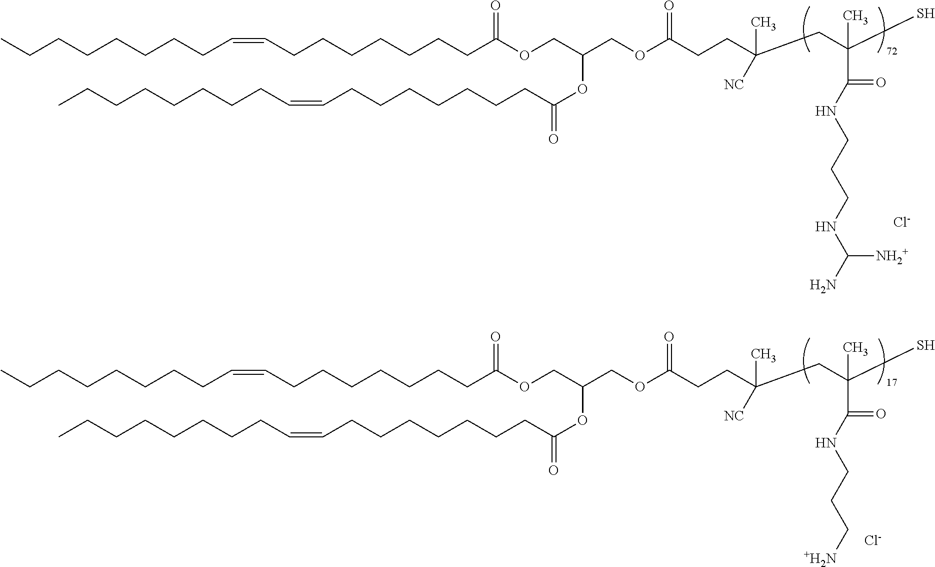

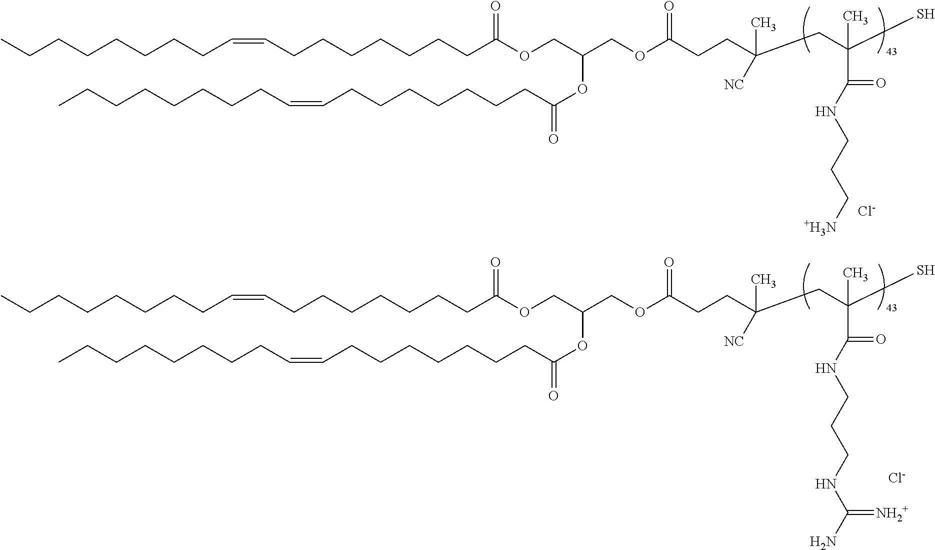

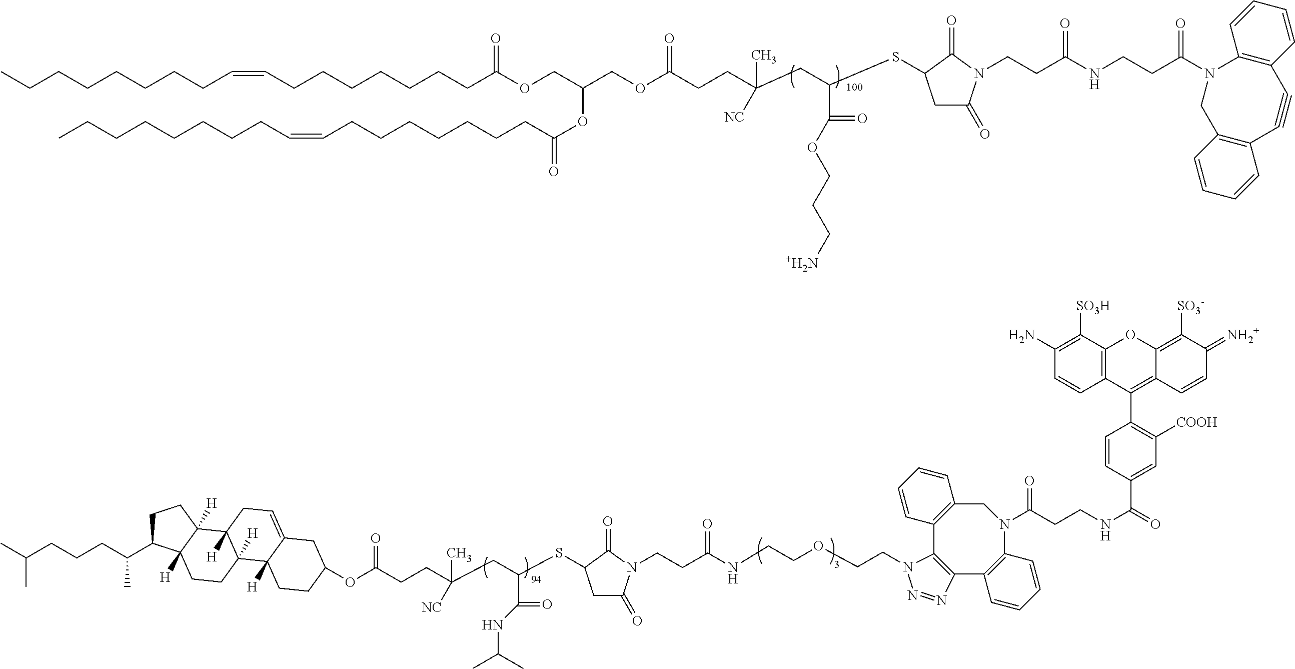

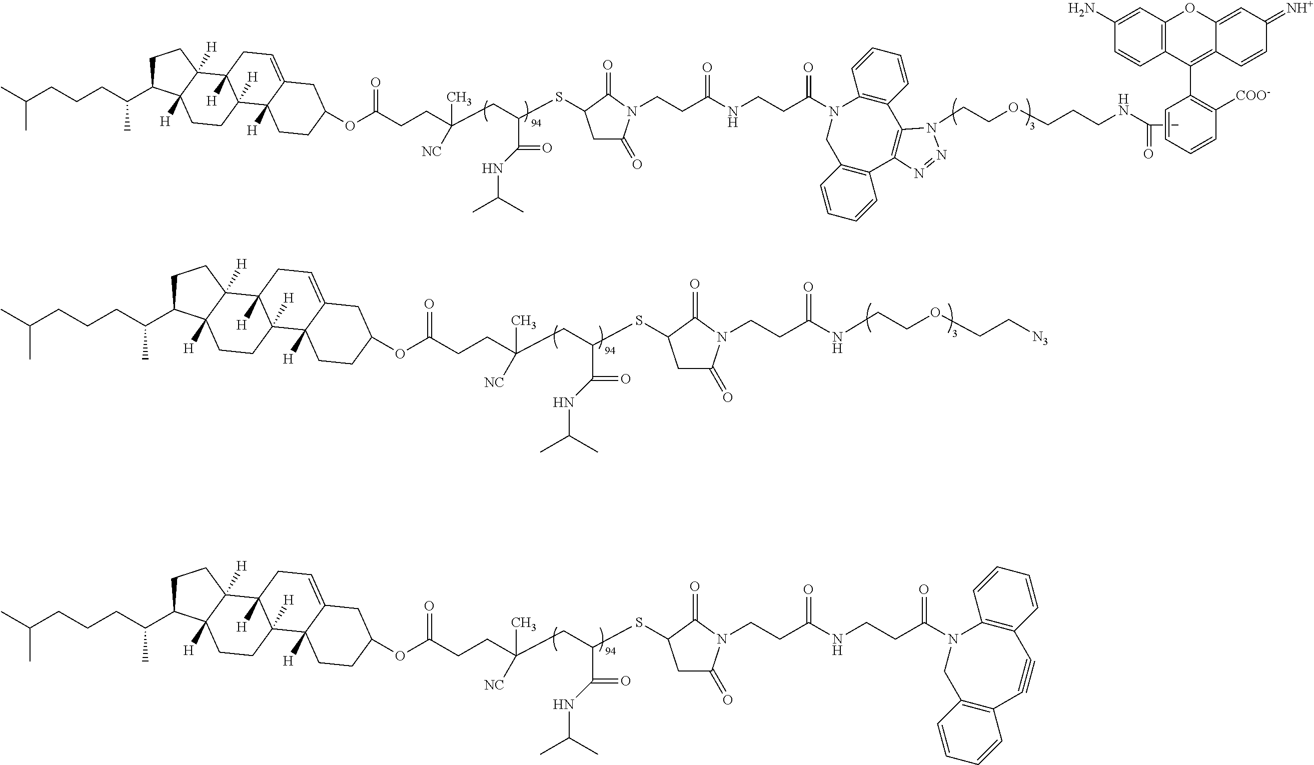

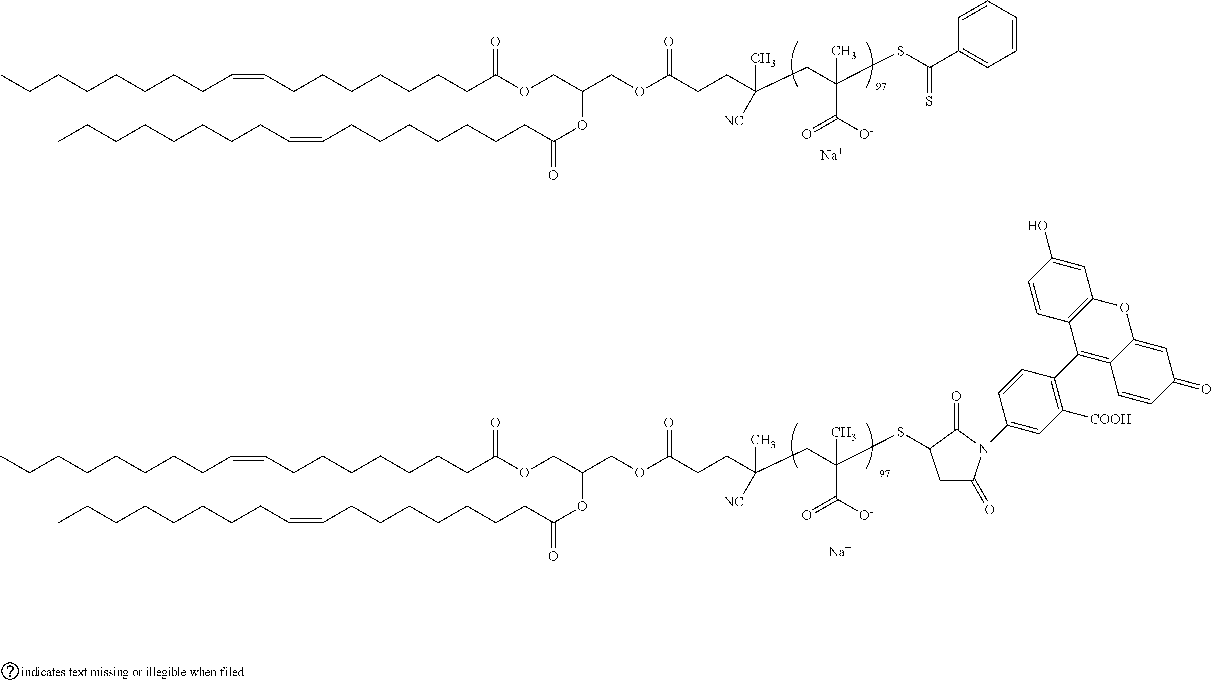

### BIOLOGY EXAMPLES

### Example B1: Encapsulation of DNA in LNPS Containing Cationic PLips

(a) Cationic PLips Incorporated into LNP Formulations for DNA Encapsulation.

Cationic PLips 1-5, with structures shown in Table B1(a), were synthesized in analogy to Synthesis Examples S1-S9. The cationic PLips were incorporated into lipid nanoparticle (LNP) formulations, and the resulting LNPs were evaluated for DNA encapsulation.

TABLE B1(a)
Structures of cationic block copolymer PLips incorporated into LNPs

| Cationic | |
|---|---|
| PLip No. | Cationic PLip Structure |
| 1 | 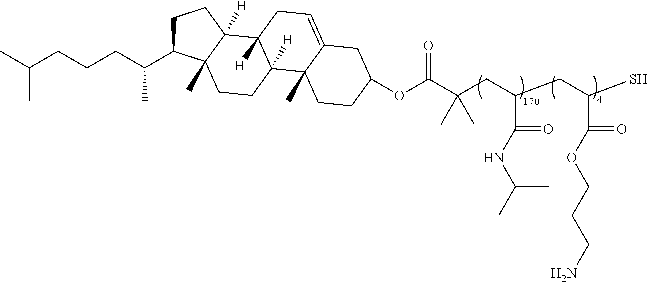 |
| 2 | 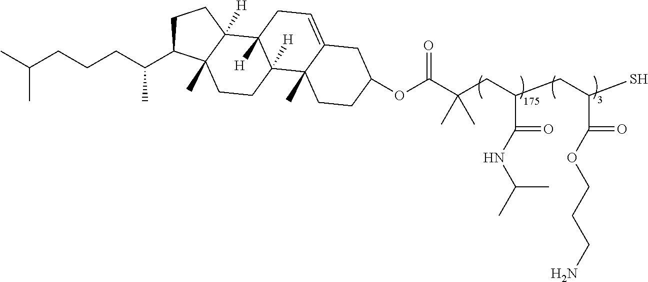 |
| 3 | 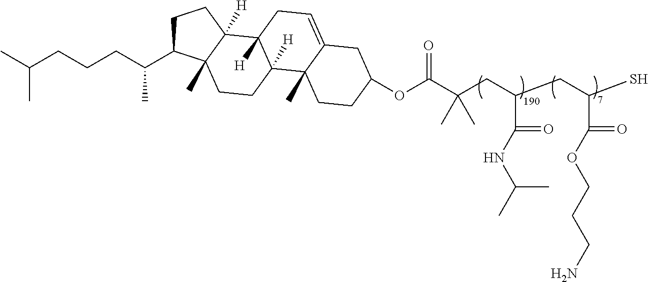 |
| 4 | 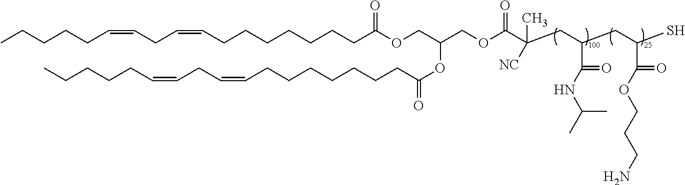 |
| 5 | 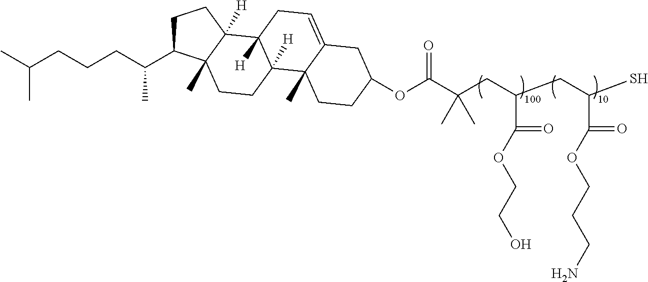 |

### (b) Formulation of LNPs Containing Cationic PLips 1-5

The cationic PLips 1-5 of Table B1(a) were incorporated into corresponding LNP formulations, referred to as LNP formulations 1-5 respectively. Each LNP comprised between about 0.3% and 1% of a given cationic PLip. By way of example, the composition of LNP formulation 1 is presented in Table B1(b). LNP formulations 2-5 were prepared according to the same specifications, replacing the PLip for the corresponding PLips 2-5 in each case.

TABLE B1(b)
Example formulation 1 of a LNP comprising cationic PLip 1:
LNP Formulation 1

| Reagent | Mol % |
|---|---|
| DLin | 54.1% |
| DOPE | 11.5% |
| Cholesterol | 32-34% |
| PEG-DMG | 0.6% |
| PLip 1 | 0.3-1.0% |

(c) Encapsulation of DNA into LNPs Containing Cationic PLips

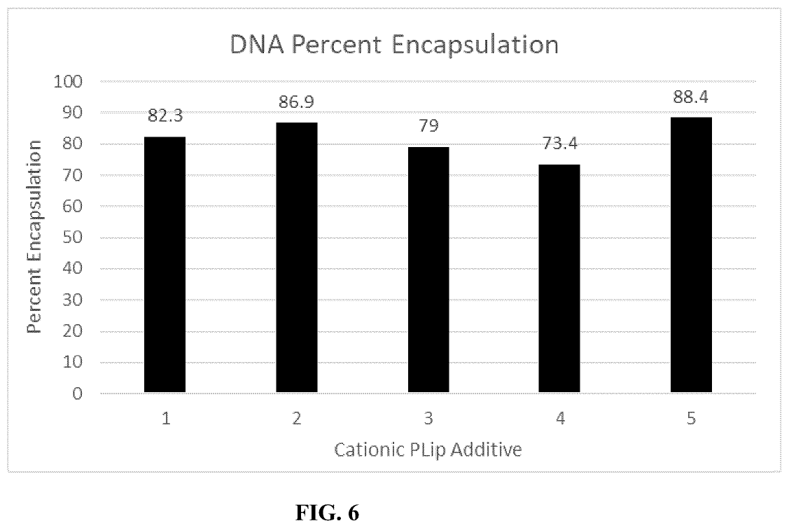

The concentration and encapsulation of DNA was determined using a modified method of the RiboQuant assay (Thermofisher). The LNPs were shown to successfully encapsulate DNA (73-88% encapsulation), indicating that the presence of the cationic PLips did not disrupt encapsulation (FIG. 6).

### Example B2: PLip-Stabilized LNPS & Effects on DNA Encapsulation

(a) PLips Incorporated into LNP Formulations for DNA Encapsulation.

PLips 6-8, with structures shown in Table B2(a) were synthesized in analogy to Synthesis Examples S1-S9. The PLips were incorporated into lipid nanoparticle (LNP) formulations, and the resulting LNPs were evaluated for DNA encapsulation.

TABLE B2(a).
Structures of stabilizing PLip 6 and cationic PLip 7

| PLip No. | Structure |
|---|---|
| 6 | 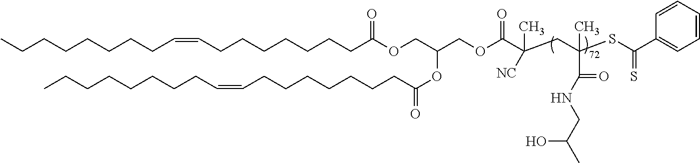 |
| 7 | 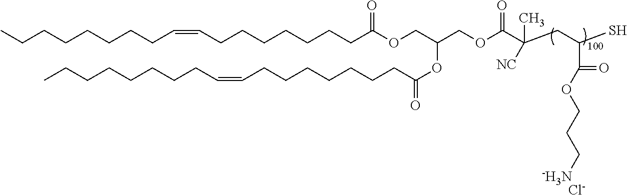 |
| 8 | 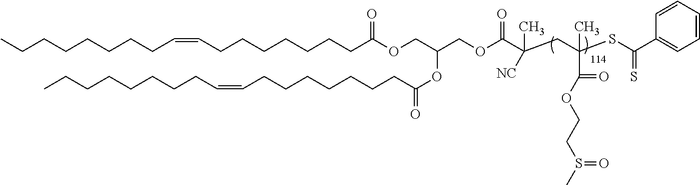 |

### (b) Formulation of L NPs Containing Stabilizing PLips 6 and 8, and Cationic PLip 7

LNPs comprising PLips 6-8 were prepared with the mol % disclosed in Table B2(b):

TABLE B2(b)
Example LNP formulations comprising PLips 6-8:

| Formulation 6 | | Formulation 7 | | Formulation 8 | |
|---|---|---|---|---|---|
| REAGENT | MOL % | REAGENT | MOL % | REAGENT | MOL % |
| DLin | 54.1% | DLin | 54.1% | DLin | 54.1% |
| DOPE | 11.7% | DOPE | 11.5% | DOPE | 11.7% |
| Cholesterol | 33.2% | Cholesterol | 32.9% | Cholesterol | 33.2% |
| PLip 6 | 1.0% | PLip 6 | 1.0% | PLip 8 | 1.0% |
| | | PLip 7 | 0.5% | | |

\textcircled{c} Encapsulation of DNA into LNPs Containing Cationic PLips

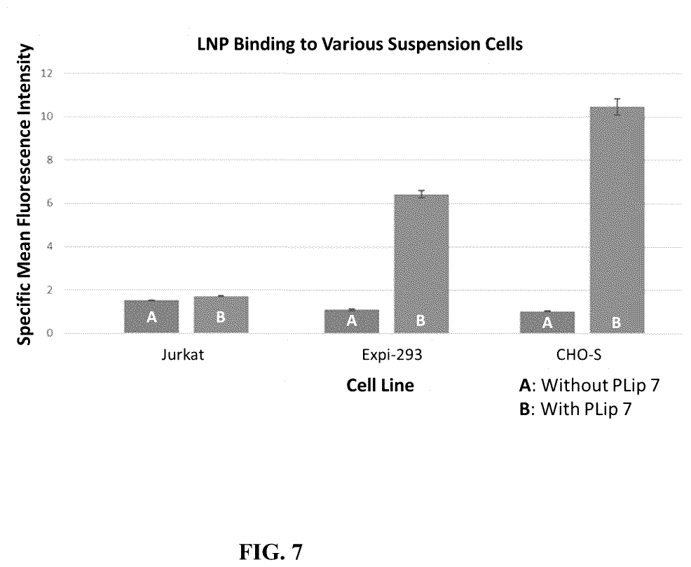

500 ng of MFP-488 labeled pDNA encapsulated in LNPs were exposed to cells for 3.5 hours at 37° C. Cells were assessed for fluorescence by flow cytometry. Specific mean fluorescence intensity (SMFI), or the cell brightness relative to untreated cells, was measured in FIG. 7. pDNA concentration and encapsulation was determined via a modified method of the RiboQuant assay (Thermofisher). Formulation 7 showed enhanced LNP binding in 2 out of 3 cell lines when compared to an identical LNP formulation without the cationic PLip 7 (shown in FIG. 7). Enhanced binding is attributed to the cationic polymer headgroup on the PLip, allowing for ionic interactions with the cell surface that would otherwise be shielded by the stabilizing, uncharged lipid.

### (d) Sizing and Stability of LNPs Stabilized by PLips

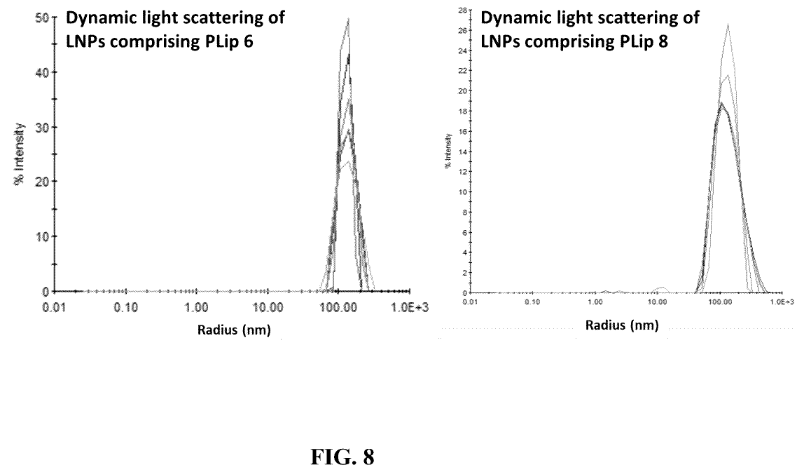

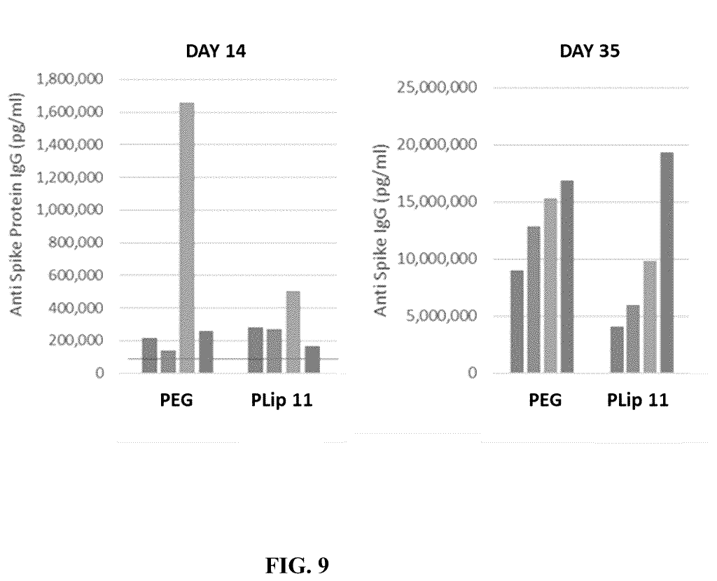

As illustrated in FIG. 7, the stabilizing PLip 7 was successfully used to stabilize and bind LNPs to cells in vitro. Dynamic light scattering (DLS) indicated well-defined stable LNPs were formed (129 nm radius and PD of 9.8% for LNP stabilized with PLip 6). Similarly, PLip 8—a PLip with a poly(MSEMA) side-chain—formed stable LNPs with a radius of 120 nm (PD=17%). The formulation of the LNP comprising PLip 8 is disclosed in Table B2(b) above. The DLS intensity distribution of LNPs comprising PLips 6 and 8 are shown in FIG. 8.

### Example B3: PLip-Stabilized LNPS & Effects on mRNA Delivery In Vivo

### (a) Synthesis and Structures of Stabilizing PLips 9-11

Additional examples of stabilizing PLips are provided in the following Table B3(a). PLips 9-11 were prepared and formulated as in the preceding examples.

TABLE B3(a).
(a). Structures of PLips 9-11

| PLip No. | Structure |
|---|---|
| 9 | 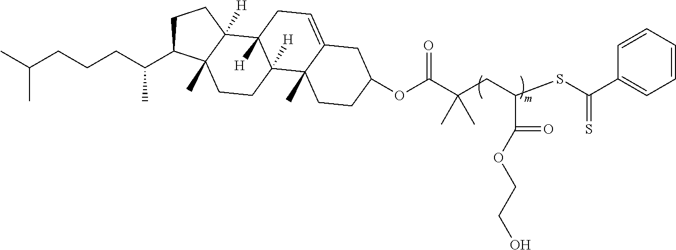 |
| 10 | 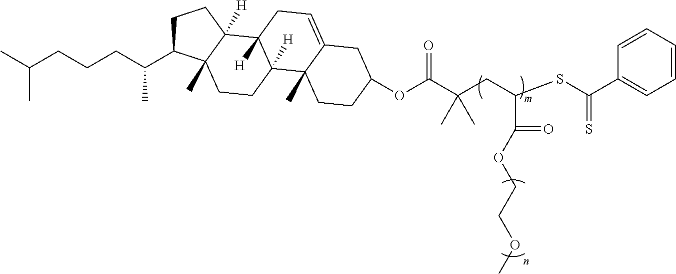 |
| 11 | 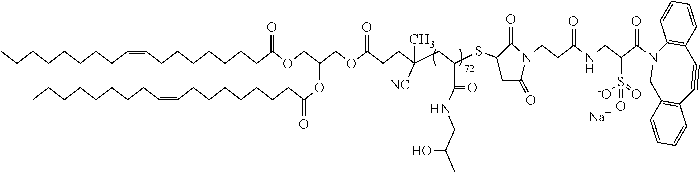 |

(b) Formulation of LNPs Containing Stabilizing PLip 11 vs. PEG-DMG

Stabilizing PLip 11 containing a diacyl tail and P(HPMA) headgroup was used in the preparation of LNP Formulation 11, according to Table B3(b). A reference LNP was prepared using PEG-DMG in place of PLip 11.

TABLE B3(b)
Example LNP formulation 11 comprising PLip 11
& reference LNP formulation comprising PEG-DMG.

| | |
|---|---|
| Formulation 11 | Reference Formulation |

| REAGENT | MOL % | REAGENT | MOL % |
|---|---|---|---|
| DLin | 54.1% | DLin | 54.1% |
| DOPE | 11.7% | DOPE | 11.7% |
| Cholesterol | 33.6% | Cholesterol | 33.6% |
| PLip 11 | 0.6% | PEG-DMG | 0.6% |

(c) In Vivo Delivery of mRNA Via LNPs Stabilized with PLips

4 μg of LNP-encapsulated mRNA samples were injected into four 20-25 g female Balb-c mice on days 1 and 21. Serum samples were assessed for anti-spike protein IgG via an ELISA on days 14 and 35. The LNP comprising PLip 11 showed a marked increase in anti-spike protein IgG via an ELISA from day 14 to day 35 that was comparable to the LNP made with PEG-DMG (FIG. 8). Each bar represents an individual mouse.

### Example B4: Cell Membrane Insertion of PLips

(a) Insertion of PLips with Fluorescent Functional Groups into a Cell Membrane

The insertion of functional and/or labelled lipids into cell membranes to ‘prime’ the membrane has been investigated for the purpose of various potential applications including the incorporation on the cell surface of sugars, functional groups (such as bio-orthogonal “click” chemistries), and fluorescent labels. To demonstrate that PLips can be inserted into the membranes of living cells, a fluorescent label-containing PLip 12 with the structure:

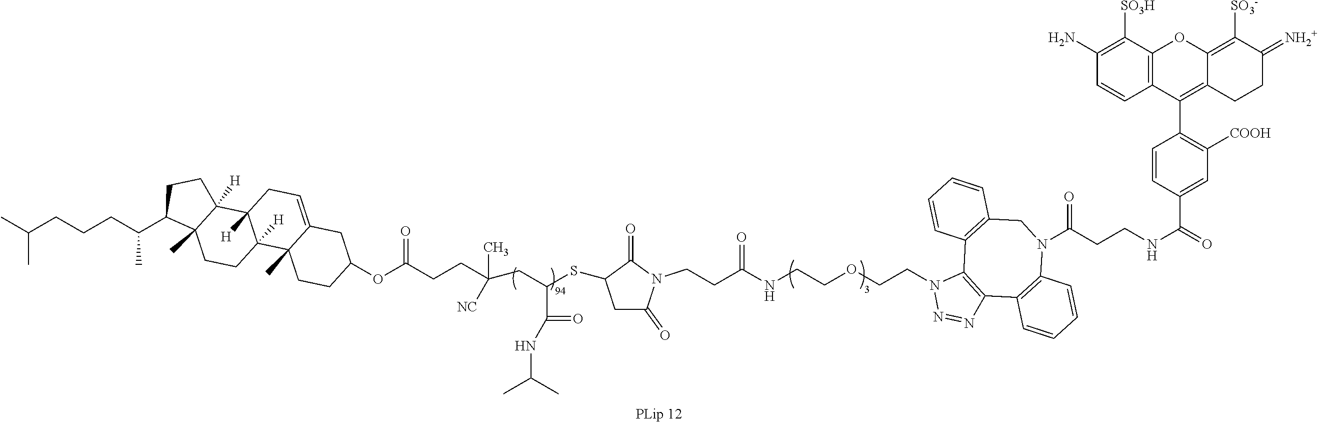

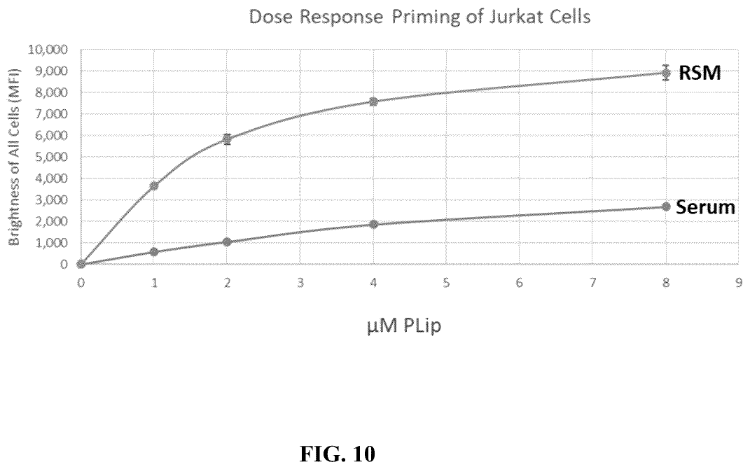

was added to Jurkat cells in either serum or reduced serum media (“RSM”)(Thermo Fisher Scientific Opti-MEM™ reduced serum medium). Increasing concentrations of the fluorescent PLip 12 were incubated with the cells for 1.5 h at 37° C., followed by 60 min at room temperature. Cells were washed once prior to fluorescence assessment via flow cytometry. The brightness of the cells was recorded as a function of the ‘priming’ concentration of the PLip (FIG. 10). The presence of serum inhibited cell priming, while priming efficiency seemed to plateau at ˜4 μM PLip under these conditions.
(b) Insertion of Temperature-Sensitive PLips with Fluorescent Side Groups into 293F Cells

A fluorescently labelled PLip 13 with the structure:

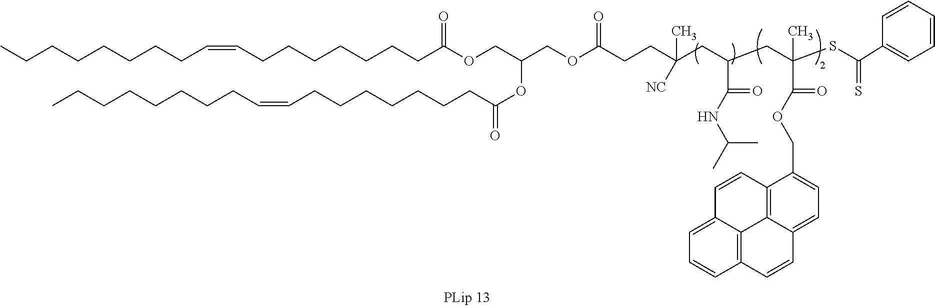

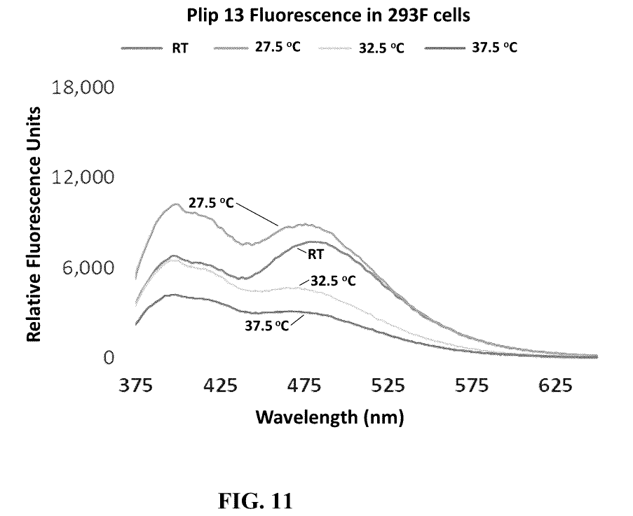

having a random copolymer headgroup of 1-pyrenemethyl methacrylate and NIPAm was incorporated into the cytoplasmic membrane of 293F cells. The PLip was incubated with cells at 12° C. to avoid endocytosis of the PLip, and then washed with phosphate-buffered saline (PBS) 3 times to remove free, unincorporated PLip. Fluorescence measurements indicated the PLip was incorporated into the cell or cell membrane (FIG. 11). Upon heating the cells from room temperature to 37.5° C., the emission peak associated with the pyrene dimer (around 480 nm) decreased in comparison with the emission peak associated with a single, monomeric pyrene (around 396 nm). The decrease in the emission of the dimer may be due to the local environment of the pyrene: for example, if pyrenes are in hydrophobic region, the pyrenes become less proximal.

293F cells (500 k cells/ml, in untreated 6-well plates) were precooled to 12.5° C. for 2 hours. Pyrene PLips were added to the cells and incubated at 12.5° C. for 1 h to allow outer membrane incorporation (in the absence of endocytosis). A 1 mL aliquot of each condition was washed 3 times in PBS. 200 μl of each condition, +/− washing, was measured on the tecan fluorescent plate reader at RT and at 37° C.

### Example 135: PLips with Bio-Orthogonal Reactive Groups at the Terminus or Along the Backbone

PLips were synthesized that contain bio-orthogonal reactive groups, which can participate in chemical reactions in a biological medium or environment (in vivo). One such example of a bio-orthogonal reactive group is a “click” chemistry group such as a strained alkyne or azide moiety. One example of a strained alkyne is the dibenzocyclooctyne (DBCO) group.

### (a) General Reaction Scheme for the End-Group Modification of a PLip.

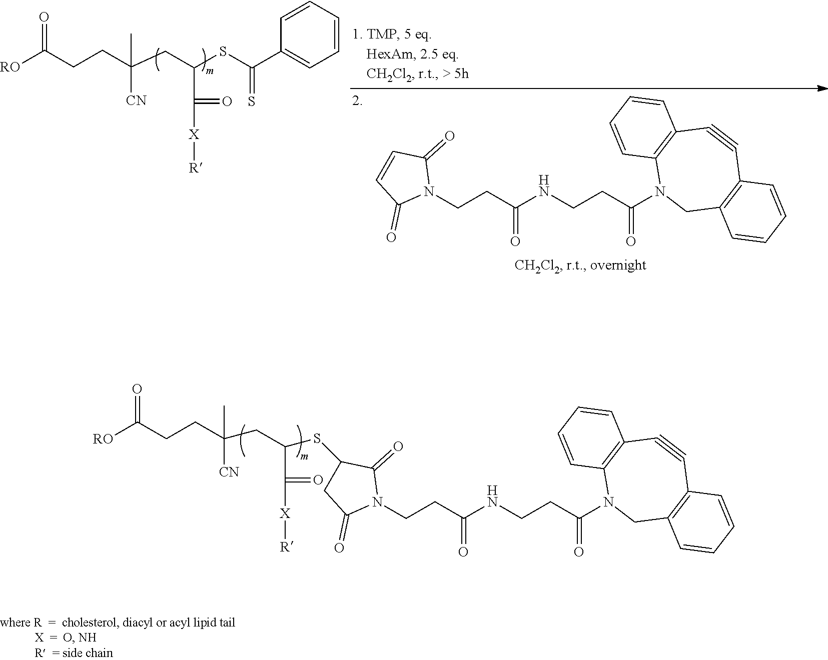

In accordance with the preceding schemes, a reactive PLip containing a bio-orthogonal DBCO functional group was incorporated in PLips 14-19, which have the following structures:

| PLip | |
|---|---|
| No. | Structure |
| 14 | 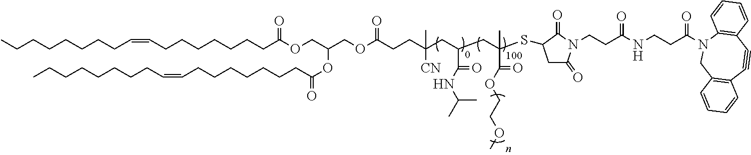 |
| 15 | 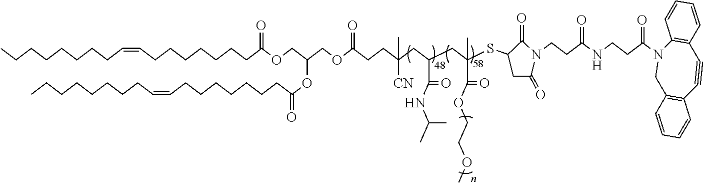 |
| 16 | 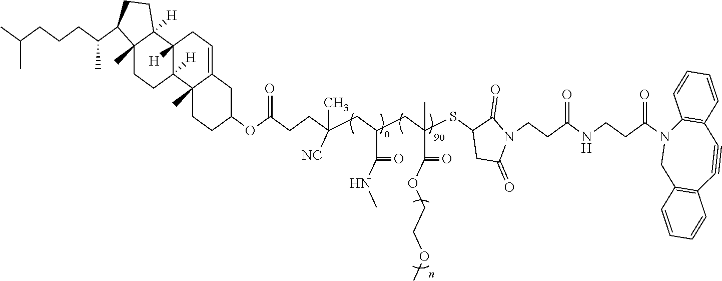 |
| 17 | 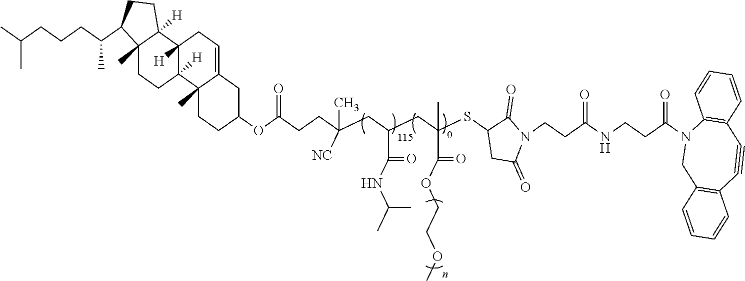 |
| 18 | 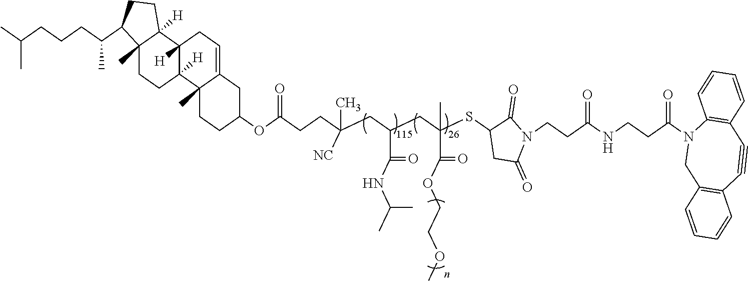 |
| 19 | 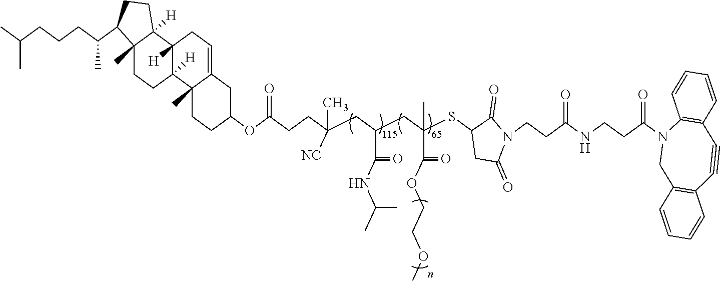 |

wherein each n is independently 0-100. More specifically, n is generally an integer from 1 to about 50. In certain examples disclosed herein, n has an average value between 2 and 10 (e.g., n=4.5).

### (b) Formulation of LNPs Containing Reactive/Stabilizing PLip 14

Stabilizing PLip 16 containing a cholesterol lipid tail, a PEG methacrylate polymer, and a DBCO functional group was used to formulate/stabilize LNPs. 1 mol % of the reactive PLip was formulated with a typical LNP mixture. One example of an LNP formulation comprising a reactive PLip, e.g., PLip 16, is LNP Formulation 16, according to Table B5 (b). Comparison is made to the same reference formulation as in Example B3 (b), copied below from Table B3 (b). The resulting LNPs according to Formulation 16 and the Reference Formulation are referred to as PLip 16 LNP and PEG LNP respectively.

TABLE B5(b)
Example LNP formulation 16 comprising PLip 16 and
reference LNP formulation comprising PEG-DMG.

| | |
|---|---|
| Formulation 16 | Reference Formulation |

| REAGENT | MOL % | REAGENT | MOL % |
|---|---|---|---|
| DLin | 54.1% | DLin | 54.1% |
| DOPE | 11.7% | DOPE | 11.7% |
| Cholesterol | 33.2% | Cholesterol | 33.6% |
| PLip 16 | 1.0% | PEG-DMG | 0.6% |

### (c) Coupling of Fluorescent Labels to PLip-Stabilized LNPs

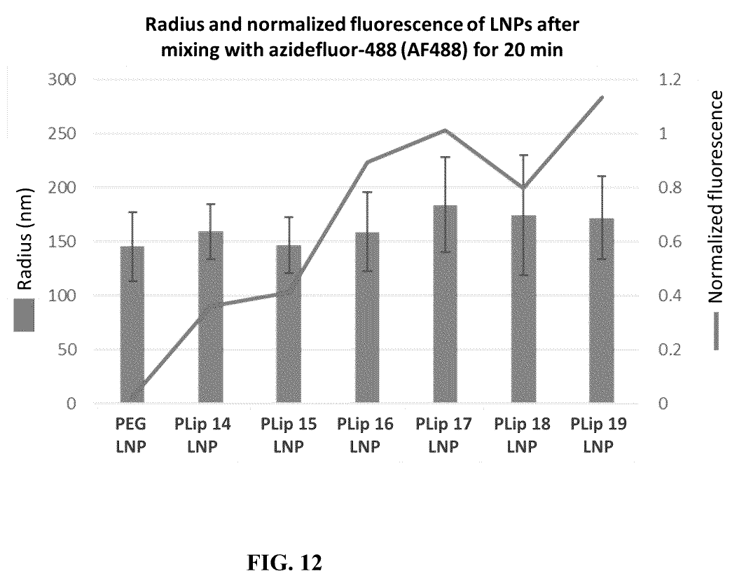

LNPs were prepared for multiple PLips having reactive (e.g., DBCO) functional groups. LNPs were analyzed by DLS prior to the addition of an excess of an azide containing fluorophore (azidefluor-488 (AF488)) for 20 min. Excess AF488 was removed via dialysis. The increased fluorescence of the LNPs (which appeared green) indicated DBCO moieties were present in LNPs and were available to undergo “click” reactions post-LNP formation (FIG. 12). The reference LNP containing 0.6% PEG2 k but no DBCO-PLip did not show any fluorescence, indicating the AF488 was covalently attached to the DBCO-LNPs.

### Example B6: Transfection Reagent In Vitro Cationic PLips Delivered Alone or Added to Formulations

### (a) Effects of N:P Ratio and Cationic Polymer Length on Transfection

293F cells were transfected with DNA (pCILuc) complexed with a variety of cationic PLips which contain a cholesterol tail and a poly(propylaminoacrylate) headgroup of 5, 13, and 19 units long at various PLip/DNA ratios. The structures are shown as below.

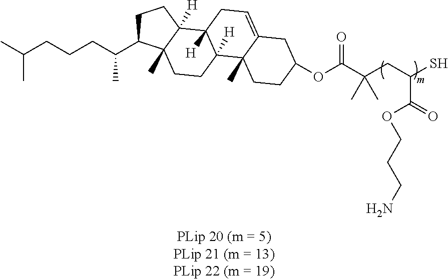

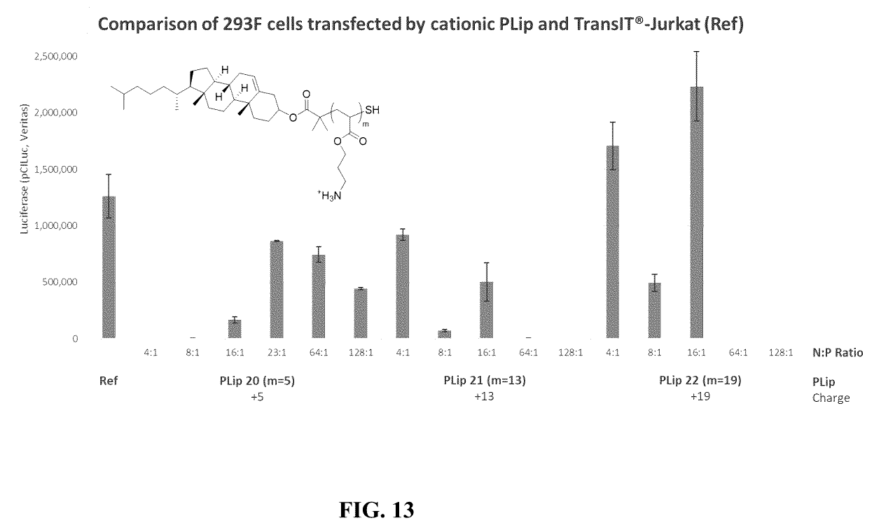

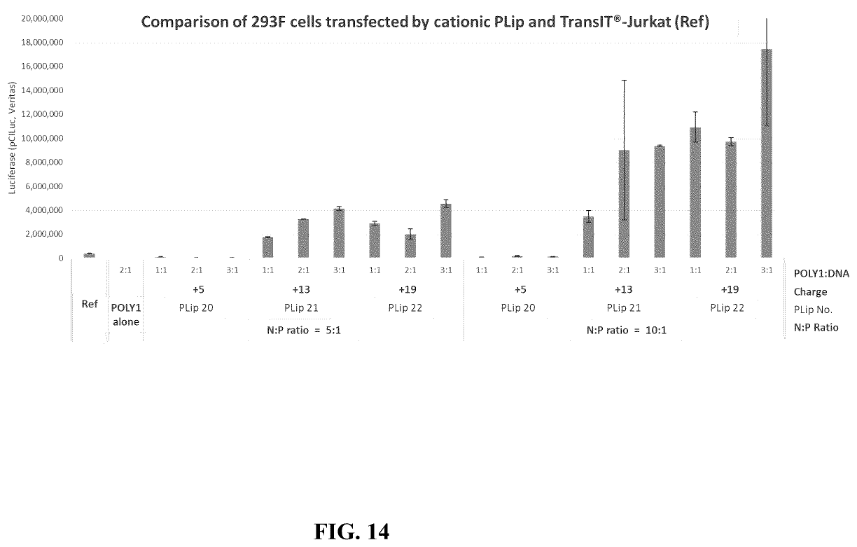

The N:P ratios were calculated from the stoichiometry of the number of cationic amines (N) on the PLip and phosphate groups (P) on the DNA. Transfection reagent TransIT®-Jurkat (Mirus Bio) was used as a comparison. Cells were transfected at various PLip:DNA (N:P) ratios for 48 hours using a CMV driven firefly luciferase pDNA construct. Cells were harvested 48 hours post-transfection by lysing the entire well with 1% triton-X 100 for 30 minutes at 4° C. Lysates were assessed for luciferase using standard conditions in a Veritas Luminometer. It was shown that some of the cationic PLips at higher N:P ratios enhanced the delivery of pCILuc as compared to TransIT®-Jurkat. In general, transfection increased at higher N:P ratios until the PLips became too toxic (FIG. 13).

### (b) Effects of N:P Ratio and Cationic Polymer Length on Delivery of a Polymer/DNA Complex

In another experiment, 293F cells were transfected with a variety of DNA (pCILuc) complexed with a proprietary polymer, POLY1, with and without cationic PLips 20-22. TransIT®-Jurkat (2:1 polymer:DNA by weight) was used as a comparison. Cells were transfected at various compound:DNA mass ratios (N:P ratios indicated as well) for 48 hours using a CMV driven firefly luciferase pDNA construct. Cells were harvested 48 hours post-transfection by lysing the entire well with 1% triton-X 100 for 30 minutes at 4° C. Lysates were assessed for luciferase using standard conditions in a Veritas Luminometer. PLip 20 did not enhance delivery of the POLY1:DNA complex. However, the cationic PLips with longer polymer head group chain lengths (PLip 21 and PLip 22) showed significant improvements in transfection (FIG. 16).

### Example B7: Temperature Sensitive PLips for (De)Stabilizing LNPS

### (a) Preparation of a Temperature-Sensitive PLip for Stabilizing LNPs

Poly(N-isopropylacrylamide)(“P(NIPAm)”) is a polymer with a lower crystalline solution temperature (LCST) of 32° C. Below the LCST, the polymer is completely soluble in water (in this instance, P(NIPAm) can be regarded as hydrophilic). Above the LCST, the polymer becomes insoluble due to intramolecular hydrogen-bonding interactions, and the polymer will aggregate/precipitate (essentially P(NIPAm) is regarded as hydrophobic). A temperature sensitive di-oleyl diglyceride PLip with P(NIPAm) side-chains and 55 monomeric units (PLip 23) was prepared according to the preceding examples, and has the following structure:

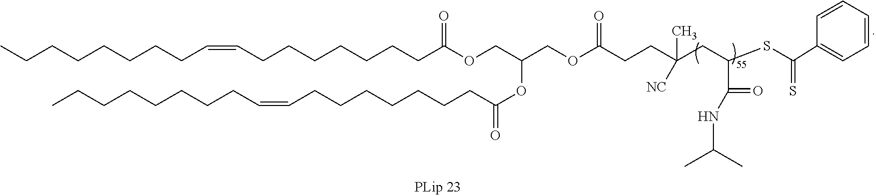

(b) Incorporation of PLip 18 into LNPs

Three LNP formulations were prepared with varying amounts of PLip 23, as disclosed in Table B6(b). Formulation 23(a) is the control and has 0 mol % PLip, whereas LNP Formulation 23(b) and 23(c) have 0.6 mol % and 1.2 mol % respectively.

TABLE B7(b)
Example LNP formulations comprising
PLip 23 at 0, 0.6, and 1.2 mol %.

| LNP | | LNP | | LNP | |
|---|---|---|---|---|---|
| Formulation 23(a) | | Formulation 23(b) | | Formulation 23(c) | |
| REAGENT | MOL % | REAGENT | MOL % | REAGENT | MOL % |
| DLin | 54.1% | DLin | 54.1% | DLin | 54.1% |
| DOPE | 11.5% | DOPE | 11.5% | DOPE | 11.5% |
| Cholesterol | 32.9% | Cholesterol | 32.3% | Cholesterol | 31.7% |
| PEG-DMG | 1.5% | PEG-DMG | 1.5% | PEG-DMG | 1.5% |
| PLip 23 | 0% | PLip 23 | 0.6% | PLip 23 | 1.2% |

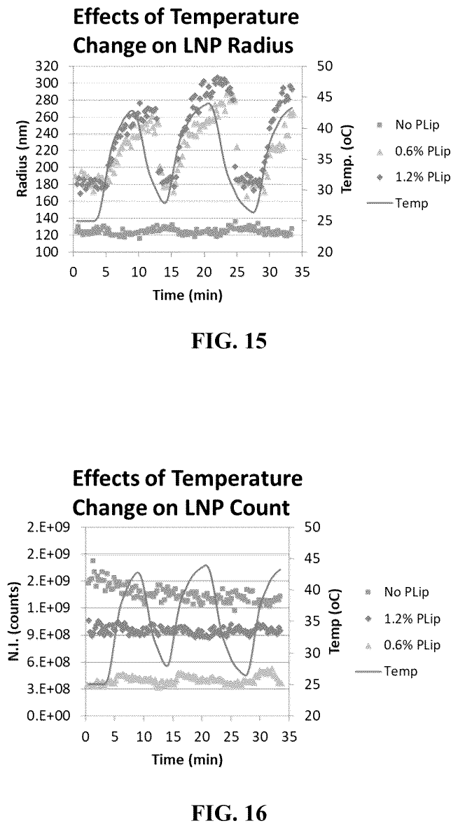

The LNPs were analyzed by DLS to determine the effects of temperature on LNP size during a cyclic temperature sweep. At room temperature (below the LCST of P(NIPAm)), the LNPs with 0.6 and 1.2 mol % PLip 23 were stable with a radius of 180 nm (FIG. 15). The LNP with no PLip, having a radius of 120 nm, was used as a control. As the temperature passed 32° C., the LNPs with the PLip started to grow. The increase in radius is due to the physical change in the P(NIPAm) chain, causing a rearrangement of lipids. As the temperature was increased to 45° C., the LNPs containing PLip 23 grew up to a radius of 300 nm. As the temperature was cycled back down to below the LCST, the LNPs shrunk down to their original sizes.

The process was shown to be reversible. As shown in FIG. 16, the normalized intensity data, relating to the number of counts of particles, did not change. These data indicate that particles were not aggregating due to the transition. The control LNPs showed no change in size or normalized intensity.

### Example C1: Synthesis of Another Temperature-Sensitive, “Stabilizing” PLip

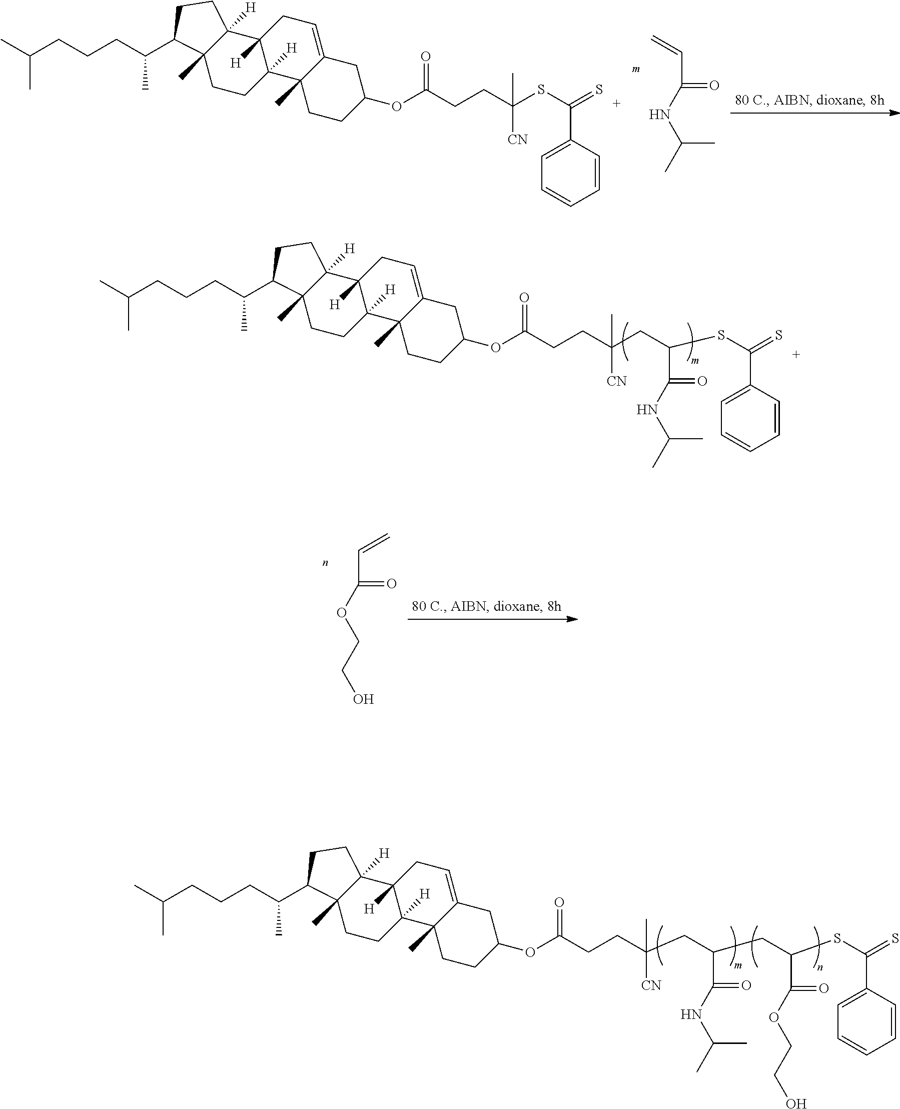

i) Preparation of Block A PLip: Add N-(isopropyl acrylamide)(800 mg, 7.08 mmol), CPCPA-cholesterol (69.3 mg, 0.107 mmol), AIBN solution (2.62 mg, 0.0160 mmol AIBN), and dioxane (4.20 mL) to a 20 mL glass vial with a septum cap and a stirrer bar. Seal the flask with the cap and bubble the solution for 30 min with nitrogen using a long needle submerged in the solution and a second needle above the solution as the outlet. Remove the syringes without removing the cap and immerse the vial into an oil bath set at 80° C. for 8 h while stirring. Allow the solution to cool to room temperature and precipitate into hexane (40 mL). Reprecipitate into hexane twice more (40 mL each time) from 2.5 mL of chloroform. Dry the collected precipitate under reduced pressure. Remove a small sample of polymer for NMR and GPC analysis to determine the length of the NIPAm chain compared to RAFT (aromatic) end group. NIPAm units=115 (m=115). Yield=566 mg (65%).

ii) Adding Block B (PHEA): Add HEA (28.0 mg, 0.241 mmol), Block A PLip obtained above (40.0 mg, 0.00294 mmol), AIBN solution (0.0980 mg, 0.000598 mmol AIBN), and dioxane (0.32 mL) to a 2 mL glass vial with a septum cap and a stirrer bar. Seal the flask with the cap and bubble the solution for 20 min with nitrogen using a long needle submerged in the solution and a second needle above the solution as the outlet. Remove the syringes without removing the cap and immerse the vial into an oil bath set at 80° C. for 8 h while stirring. Allow the solution to cool to room temperature and precipitate into hexane (15 mL). Reprecipitate into hexane twice more (15 mL each time) from 1 mL of ethanol. Dry the collected precipitate under reduced pressure. Remove a small sample of polymer for NMR and GPC analysis to determine the length of the HEA chain compared to Block A. HEA units=92 (n=92). Yield=58 mg (85%).

### Example D1: Stabilized Complexes Over Time

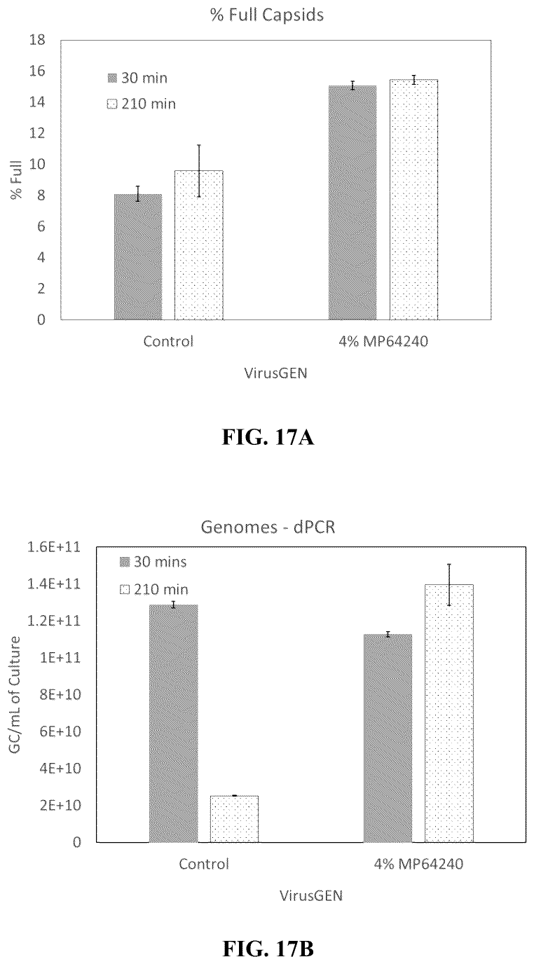

Effect of a stabilizing PLip (MP64240) on the functional performance of transfection complex (e.g., VirusGEN®) over time is analyzed: percent full capsids (FIG. 17A) and genomes (FIG. 17B). 293-VP 2.0 cells in virus production media (VPM) are seeded at 3 million cells/mL immediately prior to transfection. TransIT-VirusGEN® complexes with or without temperature-sensitive PLip are formed in phosphate-buffered saline (PBS) and measured at 30 minutes and 3.5 hours for complex formation. Plasmid: pMIR699, pMIR 732 AAV8, and pMIR701 AAV harvested 72 hrs. post transfection. Genome count/mL of culture is measured via digital PCR (dPCR). AAV8 capsids are measured via Lumit® AAV Capsid Immunoassay. Stabilization of VirusGEN® over 3.5 h in terms of the genome titer is shown in FIG. 17B in the presence of temperature-sensitive PLip, which is added to the transfection reagent in ethanol prior to mixing with plasmid DNA.

### Example D2: Stabilization of Concentrated Transfection Complexes

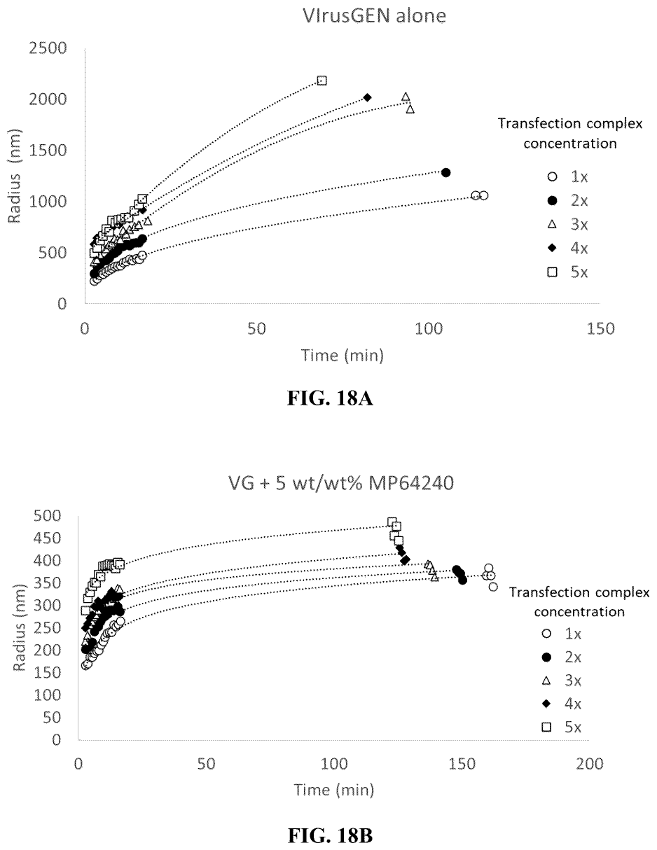

Dynamic Light Scattering (DLS) is used to follow the aggregation behavior of transfection complexes over time. The stabilizing effect of adding a 5% wt/wt (compared to the amount of lipid in VirusGEN®) of temperature-sensitive PLip (MP64240) to concentrated (1 to 5 times the normal (recommended) concentrations of VG and DNA in PBS) is shown in FIG. 18A and FIG. 18B. 2× and 5× denote that the concentration of VirusGEN® and DNA is twice and five times that of the recommended protocol. The presence of temperature-sensitive PLip stabilizes the transfection complexes that would have been over a micron in diameter after only a few minutes (without the PLip) to under 1 micron in diameter over >2 hours (with the PLip).

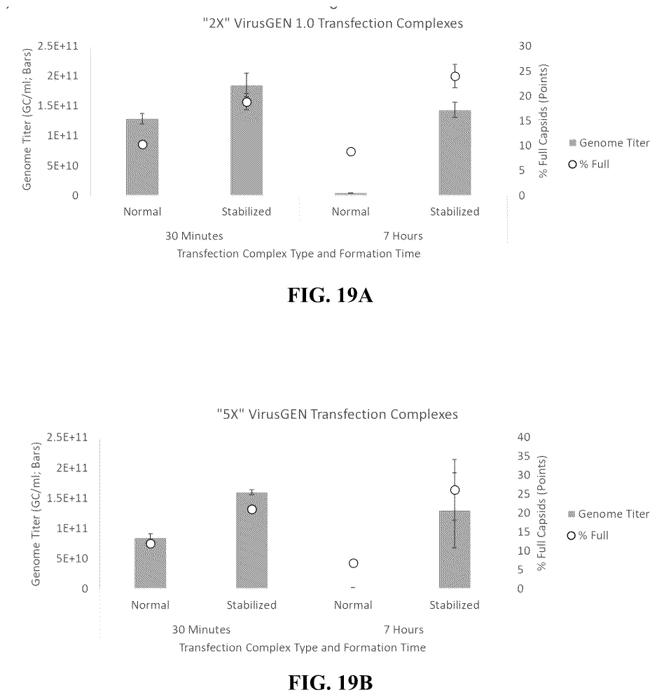

The functional performance of 2× and 5× VirusGEN® complexes with and without a stabilizing PLip (such as MP64240) is determined. FIG. 19A and FIG. 19B highlight the variations in terms of the total genome titers and percent full capsids of the “Normal” unstabilized and the stabilized complexes at 30 min and 3.5 h. At the 30 min time point, the 2× complex shows similar results to a standard VirusGEN (1×) complex in terms of titers and percent full. The 5× complex is showing lower titers and similar percent full, likely due to the larger size of the complex at this timepoint. At 3.5 h both the 2× and 5× complexes that are not stabilized show very little functional efficacy. In contrast, when the 2× and 5× complexes are stabilized with the temperature-sensitive PLip, functional performance is maintained across a longer time and for more concentrated complexes (2× and 5×).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and (heir equivalents be covered thereby.

1. A compound comprising:
(a) a lipid; and
(b) a stimulus-responsive unit.

2. The compound of claim 1 further comprising:
(c) a linker comprising said stimulus-responsive unit; and
(d) a polymer, the polymer having a backbone,
wherein said linker connects said lipid to said backbone of said polymer.

3. The compound of claim 2, wherein said polymer comprises at least 3 monomeric units, wherein said at least 3 monomeric units comprise a C₁₋₂₀ heteroalkyl side-chain.

4. The compound of claim 2, wherein said stimulus-responsive unit is a temperature-responsive unit, a pH-responsive unit, a light-responsive unit, or a chemical-responsive unit.

5. The compound of claim 4, wherein said temperature-responsive unit comprises a lower crystalline solution temperature (LCST) from about 27° C. to about 35° C., preferably about 27° C. about 28° C. about 29° C. about 30° C. about 31° C. about 32° C. about 33° C., about 34° C., or about 35° C.

6. (canceled)

7. The compound of claim 5, wherein said temperature-responsive unit comprises poly(N-isopropylacrylamide), poly(N-n-propylacrylamide), poly(N-methyl-N-n-propylacrylamide), poly(N,N-diethylacrylamide), poly(N-isobutylacrylamide), poly(N-sec-butylacrylamide), poly(N-n-butylacrylamide), poly(N-isobutylacrylamide), hydroxypropylcellulose, poly(N-vinylcaprolactam), poly-2-isopropyl-2-oxazoline, or polyvinyl methyl ether, or a combination thereof.

8. The compound of claim 7, wherein said temperature-responsive unit comprises 2-250 monomeric units.

9. A compound comprising a lipid connected to a backbone of a polymer, said polymer comprising at least 3 monomeric units, wherein said at least 3 monomeric units comprise a C₁₋₂₀ heteroalkyl side-chain.

10. The compound of claim 9, wherein said polymer comprises 4 or more said monomeric units, about 10 or more said monomeric units, about 50 or more said monomeric units, about 400 or less said monomeric units, about 300 or less said monomeric units, or about 10 to about 200 said monomeric units.

11. (canceled)

12. (canceled)

13. (canceled)

14. (canceled)

15. (canceled)

16. (canceled)

17. The compound of claim 9, wherein said polymer comprises a polyacrylate or a polyacrylamide.

18. (canceled)

19. The compound of claim 9, wherein said polymer is not a peptide.

20. (canceled)

21. The compound of claim 9, wherein said polymer is a copolymer.

22. The compound of claim 21, wherein said copolymer is a block copolymer, a block comprising a cationic monomeric unit, a block copolymer is a random block copolymer, or a combination thereof.

23. (canceled)

24. (canceled)

25. The compound of claim 9, wherein said polymer is positively charged in neutral aqueous solution, preferably having a pK_{b} from about 2 to about 12, more preferably having a pK_{b} from about 4 to about 11.

26. (canceled)

27. (canceled)

28. The compound of claim 9, wherein said polymer comprises: 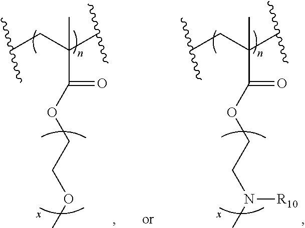
wherein:
R₁₀ is hydrogen or C₁-C₆ alkyl, and
x is an integer of 1-20.

29. A compound according to Formula I: 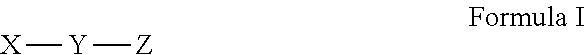
or a pharmaceutically acceptable salt thereof; wherein:
X is a lipid;
Y is a polymer comprising 3 or more monomeric units, wherein each of said monomeric unit comprises a C₁₋₂₀ heteroalkyl side-chain; and
Z is an unsubstituted or substituted functional group;
wherein said lipid is covalently bonded to said polymer via a backbone of said polymer.

30. The compound of claim 29, wherein said lipid comprises a steroid, preferably a sterol, preferably cholesterol, or a stanol, or a fatty acid, a saturated fatty acid, a monounsaturated fatty acid, a Polyunsaturated fatty acid, or a combination thereof, more preferably an oleic acid, or an ester thereof.

31. (canceled)

32. (canceled)

33. (canceled)

34. (canceled)

35. (canceled)

36. The compound of claim 29, wherein said lipid is hydrophobic or amphiphilic.

37. (canceled)

38. The compound of claim 29, wherein said lipid has an octanol:water coefficient (log(K_{OW})) of about 2 or more.

39. The compound of claim 29, wherein said lipid has a structure of Formula X-A, Formula X-B, or Formula X-C:

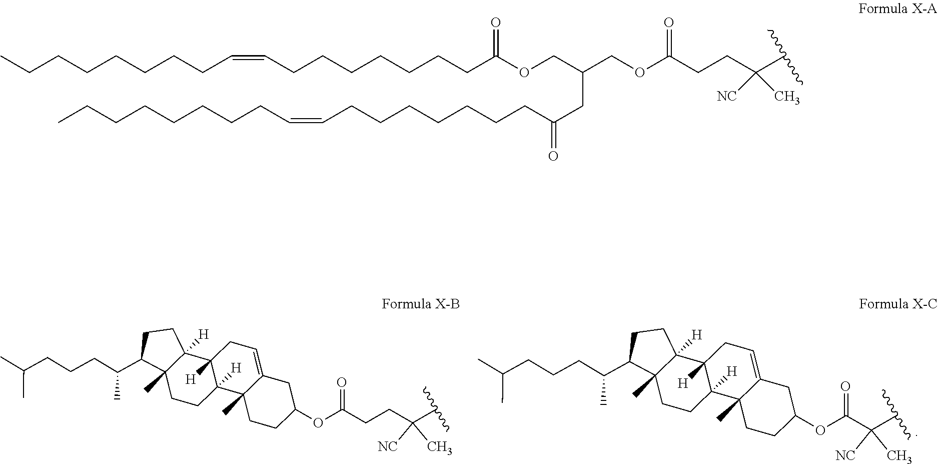

40. The compound of claim 29, wherein said polymer has a structure of Formula Y-A, Formula Y-B, Formula Y-C, or Formula Y-D: 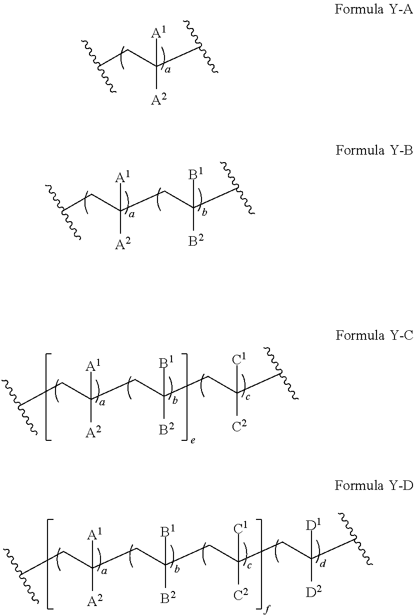
or a pharmaceutically acceptable salt thereof; wherein:
each of A¹, B¹, C¹, and D¹ is independently hydrogen or methyl;
each of A², B², C², and D² is independently unsubstituted or substituted C₁₋₂₀ heteroalkyl;
each of a, b, c, d, e, and f is independently an integer from 0 to 200, provided that the total number of said monomeric units is 3 or more;
wherein each said substituted C₁₋₂₀ heteroalkyl is independently substituted with a cycle that is an unsubstituted or substituted cycloalkyl, unsubstituted or substituted heterocyclyl, unsubstituted or substituted aryl, or unsubstituted or substituted heteroaryl.

41. The compound of claim 29, wherein said polymer comprises from about 4 to about 400 said monomeric units, preferably from about 10 to about 200 said monomeric units, more preferably, from about 50 to about 150 said monomeric units.

42. (canceled)

43. (canceled)

44. The compound of claim 29, wherein said polymer comprises a polyacrylate or a polyacrylamide.

45. The compound of claim 40, wherein each of said A², B², C², and D² independently comprises an acrylate or an acrylamide.

46. The compound of claim 29, wherein each said monomeric unit has the structure of one of the following formulae: 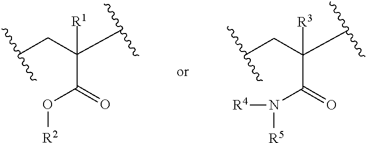
or a pharmaceutically acceptable salt thereof; wherein:
each of R¹ and R³ is independently hydrogen or methyl;
each R² is hydrogen, C₁₋₆ alkyl, C₇₋₂₀ aralkyl, C₁₋₂₀ heteroalkyl, or polyethylene glycol chain containing 1 to 100 ethylene glycol monomers; wherein each of said C₁₋₆ alkyl, C₇₋₂₀ aralkyl, and C₁₋₂₀ heteroalkyl is unsubstituted or substituted with one or more groups, wherein each of said one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, or —S(O)₂OH;
each of R⁴, and R⁵ is independently hydrogen, C₁₋₆ alkyl, C₇₋₂₀ aralkyl, or C₁₋₂₀ heteroalkyl; wherein each of said C₁₋₆ alkyl, C₇₋₂₀ aralkyl, and C₁₋₂₀ heteroalkyl is unsubstituted or substituted with one or more groups, wherein each of said one or more groups is independently —COOH, —CONH₂, —NH₂, —NH₃⁺, —NHC(NH₂⁺)NH₂, —NHCH₃, —N(CH₃)₂, —N(CH₃)₃⁺, —OH, —OCH₃, —SH, —S(O)CH₃, —S(O)₂CH₃, or —S(O)₂OH; or a pharmaceutically acceptable salt thereof.

47. The compound of claim 29, wherein each said monomeric unit independently comprises:

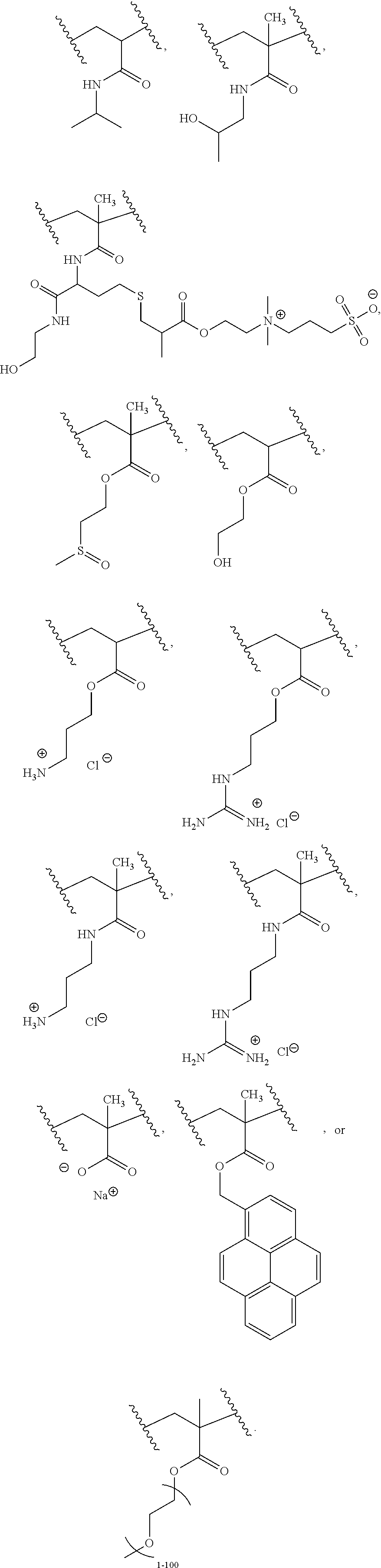

48. The compound of claim 29, wherein said functional group is a thiol or a sulfide.

49. (canceled)

50. (canceled)

51. The compound of claim 48, wherein said sulfide is SR⁶, and wherein R⁶ is a group consisting of 1 to about 200 atoms selected from hydrogen, halogen, C, N, O, and S.

52. The compound of claim 48, wherein said sulfide is SR⁶, and wherein R⁶ comprises a reactive group, a charged group, a detectable group, a peptide group, a capping group, or a combination thereof.

53. The compound of claim 52, wherein said reactive group, if present, comprises an azide or an alkyne; wherein said charged group, if present, comprises one or more cationic groups; wherein the one or more cationic groups comprise a cyclic amine, primary amine, guanidine, or a combination thereof; wherein said detectable group, if present comprises a fluorophore, a dye, a FRET donor or acceptor; and wherein said capping group, if present is an inert group.

54. (canceled)

55. (canceled)

56. (canceled)

57. (canceled)

58. The compound of claim 29, wherein said functional group is selected from: 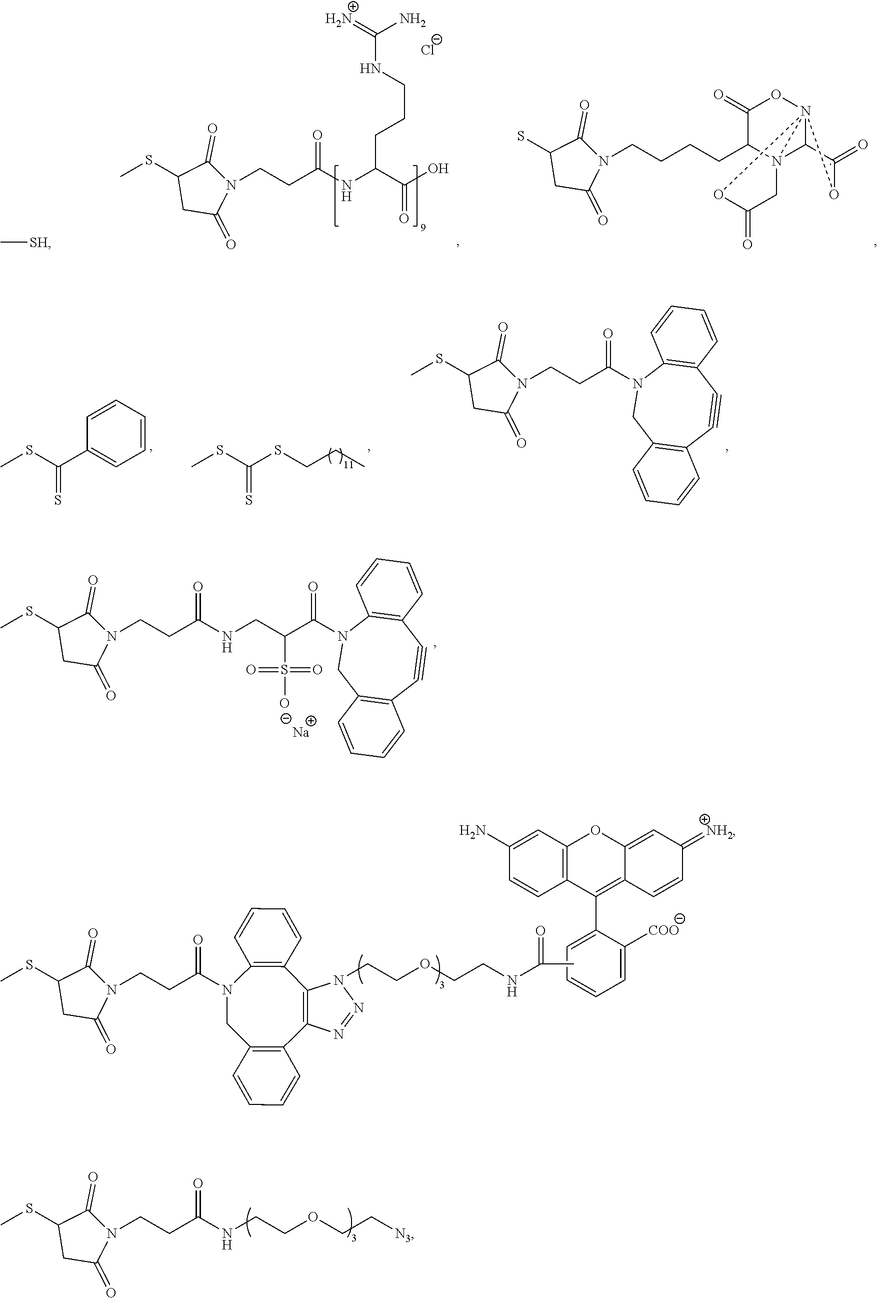 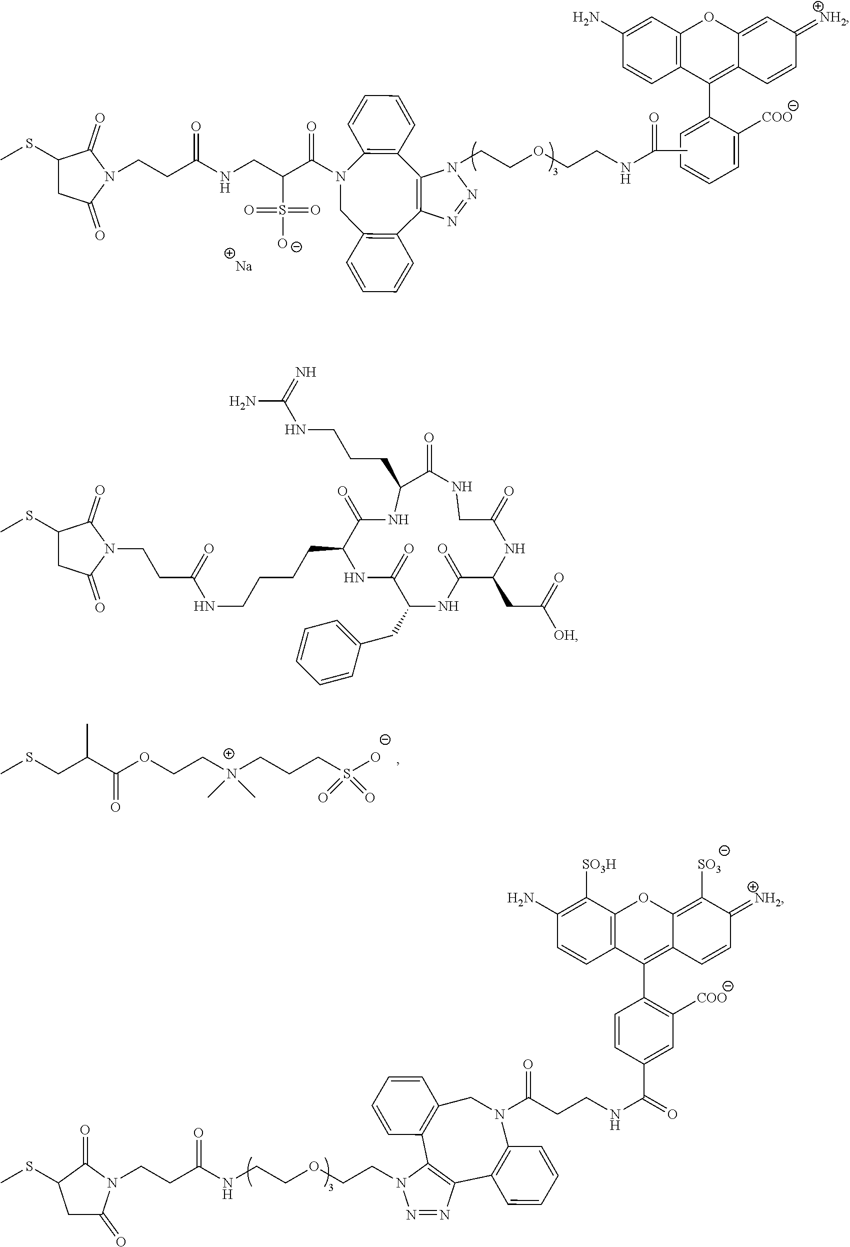 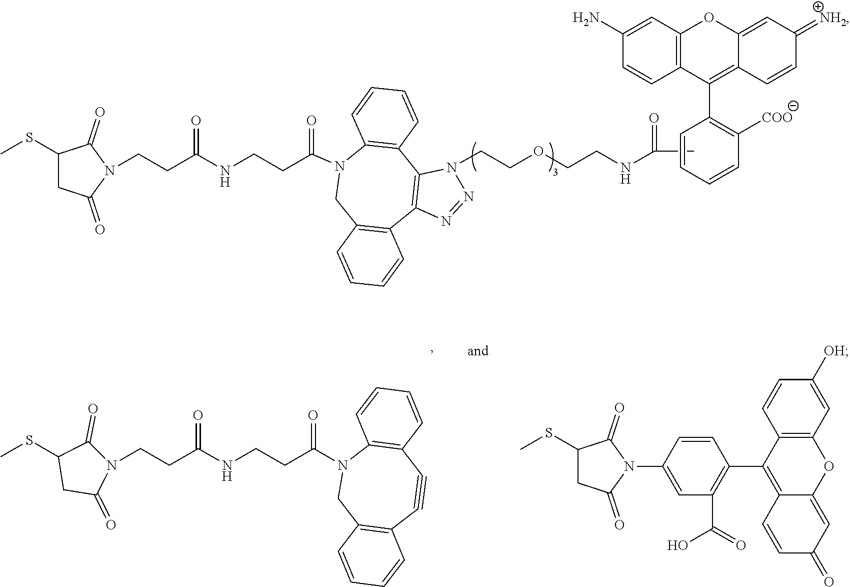
wherein said functional group is bonded to said polymer via a sulfur atom.

59. The compound of claim 29, wherein said compound is configured to encapsulate or complex with nucleic acids in aqueous solution.

60. The compound of claim 29, wherein said compound is substantially non-toxic.

61. The compound of claim 29, wherein said compound is biodegradable.

62. The compound of claim 29, wherein said compound comprises a molecular weight from about 1 kilodaltons (kDa) to about 100 kDa.

63. A nanoparticle comprising a compound of claim 1, wherein said nanoparticle is configured for encapsulation or complexation of a nucleic acid.

64. The nanoparticle of claim 63, wherein said nanoparticle is configured for said encapsulation or said complexation of said nucleic acids at a 0.3:1 to 100:1 (weight:weight) ratio.

65. The nanoparticle of claim 63, wherein said encapsulation or said complexation of said nucleic acids increases a half-life of said nucleic acids by at least 2-fold in aqueous or physiological conditions.

66. The nanoparticle of claim 63, wherein nuclease digestion of said nucleic acid is inhibited by said encapsulation or complexation.

67. The nanoparticle of claim 63, wherein said encapsulation or said complexation of said nucleic acids generates a transfection reagent with an average size of about 20 nm to about 2000 nm.

68. The nanoparticle of claim 63, wherein said complexation comprises adsorption of at least a subset of said nucleic acids to a surface of said nanoparticle.

69. The nanoparticle of claim 63, wherein said encapsulation or said complexation of said nucleic acids generates a transfection reagent configured for cellular uptake, optionally wherein said cellular uptake comprises endocytosis.

70. (canceled)

71. A transfection reagent comprising the nanoparticle of claim 63, and a nucleic acid encapsulated therein, wherein optionally said nucleic acid comprises deoxyribonucleic acid (DNA), ribonucleic acid (RNA), locked nucleic acid (LNA), peptide nucleic acid (PNA), or any combination thereof.

72. (canceled)

73. The transfection reagent of claim 71, wherein said nucleic acid comprises about 1 kilobase pairs (kb) to about 100 kb, preferably about 2 kb to about 20 kb, more preferably about 5 kb to about 15 kb, and even more preferably about 8 kb to about 12 kb, and even more preferably about 10 kb.

74. (canceled)

75. (canceled)

76. (canceled)

77. (canceled)

78. The transfection reagent of claim 71, comprising an aqueous solubility of at least 5 μg/mL, preferably about 5 μh to about 5 mg/mL and more preferably about 10 μg/mL to about 50 μg/mL.

79. (canceled)

80. (canceled)

81. A method for transfecting a cell, said method comprising:
(a) providing a transfection reagent comprising a compound of claim 1, and a nucleic acid, and
(b) contacting said cell with said transfection reagent, wherein said contacting is under conditions suitable for entry of said nucleic acid into said cell.

82. The method of claim 81, wherein (a) comprises contacting said compound with said nucleic acid under conditions sufficient to form said transfection complex, optional wherein said conditions sufficient to form said transfection complex comprise conditions sufficient for ionotropic gelation.

83. (canceled)

84. The method of claim 81, wherein said nucleic acid comprises deoxyribonucleic acid (DNA), ribonucleic acid (RNA), locked nucleic acid (LNA), peptide nucleic acid (PNA), or any combination thereof.

85. The method of claim 81, wherein said transfection complex comprises a positive charge under said conditions suitable for entry of said nucleic acid into said cell.

86. The method of claim 81, wherein said contacting is for less than 24 hours.

87. The method of claim 81, wherein said cell comprises an animal cell, a plant cell, a fungal cell, a bacterial cell, or any combination thereof.

88. A pharmaceutical composition comprising the nanoparticle of claim 63 and a bioactive molecule.

89. The pharmaceutical composition of claim 88, wherein said nanoparticle is covalently bonded to said bioactive molecule and/or is ionically bonded to said bioactive molecule.

90. (canceled)

91. The pharmaceutical composition of claim 88, wherein said nanoparticle encapsulates said bioactive molecule.

92. The pharmaceutical composition of claim 88, wherein said bioactive molecule comprises a nucleic acid molecule, preferably RNA or DNA, more preferably mRNA, siRNA, or tRNA.

93. (canceled)

94. (canceled)

95. The pharmaceutical composition of claim 88, wherein said bioactive molecule comprises a therapeutic agent, preferably a chemotherapeutic, a radiotherapeutic, an oligonucleotide, or an oligopeptide, and optionally further comprising a pharmaceutically acceptable excipient.

96. (canceled)

97. (canceled)

98. A method of treating a condition or disease in a subject in need thereof, comprising administering to said subject a therapeutically effective amount of said pharmaceutical composition of claim 88, optionally wherein said pharmaceutical composition is administered to said subject by injection.

99. (canceled)
